(19) United States
(12) Patent Application Publication
(10) Pub. No.: US 2026/0355236 A1
(43) Pub. Date: Nov. 12, 2026

(54) METHODS FOR THE GENERATION OF A MONOCYTE/MACROPHAGE CELL PRODUCT FROM EXPANDED CORD BLOOD CD34+ HEMATOPOIETIC STEM AND PROGENITOR CELLS

(71) Applicant: Deverra Therapeutics, Inc., Seattle, WA (US)

(72) Inventors: Colleen Delaney, Seattle, WA (US); Erika von Euw, Los Angeles, CA (US); Shannon Kreuser, Seattle, WA (US); Carrie Stoltzman, Seattle, WA (US); Katherine Brempelis, Seattle, WA (US)

(73) Assignee: Deverra Therapeutics, Inc., Seattle, WA (US)

(21) Appl. No.: 19/109,099

(22) PCT Filed: Aug. 30, 2023
(86) PCT No.: PCT/US2023/073151

§ 371 Date: Mar. 5, 2025

Related U.S. Application Data
(60) Provisional application No. 63/404,486, filed on Sep. 7, 2022.

(51) Int. Cl. A61K 35/15; A61K 31/711; A61K 35/768; A61K 40/17; A61K 40/24; A61K 40/31; A61K 40/32; C07K 14/705; C12N 5/00; C12N 5/0786; C12N 15/113

(52) U.S. Cl. CPC A61K 35/15; A61K 31/711; A61K 35/768; A61K 40/17; A61K 40/24; A61K 40/31; A61K 40/32; C07K 14/7051; C12N 5/0087; C12N 5/0645; C12N 15/113; C07K 2317/53; C07K 2319/02; C07K 2319/03; C12N 2501/22; C12N 2501/2303; C12N 2501/26; C12N 2501/42; C12N 2506/11; C12N 2510/00; C12N 2533/52

(57) ABSTRACT

The present disclosure provides an ex vivo method for producing a therapeutic monocyte and/or macrophage (monocyte/macrophage) composition or product for use in immunotherapy. The method comprises three phases including a hematopoietic stem cell and hematopoietic stem and progenitor (HSPC) expansion phase, an expansion and monocyte differentiation phase and a macrophage differentiation phase. The monocyte and/or macrophage composition or product can be used directly in the formulation of a therapeutic product for administration to a subject or cryopreserved for later use and/or formulation. The monocyte and/or macrophage cell product, composition and/or product can also be engineered to comprise a receptor (such as a CAR, TCR, chimeric receptor, or other receptor), an immune cell engager, an antibody, a nanobody, a transcription factor, a cytokine, and/or a chemokine. The function of the cell product also can be improved by pre-loading the cells with an oncolytic virus or other immunomodulatory molecule for delivery to a solid tumor.

### CROSS-REFERENCE TO RELATED APPLICATION

This application claims the benefit of U.S. Provisional Application No. 63/404,486, filed Sep. 7, 2023, the disclosure of which is incorporated herein by reference in its entirety.

### STATEMENT REGARDING SEQUENCE LISTING

The Sequence Listing XML associated with this application is provided in XML format and is hereby incorporated by reference into the specification. The name of the XML file containing the sequence listing is 1765-P13WO_Seq_List_20230829.xml. The XML file is 90,003 bytes; was created on Aug. 29, 2023; and is being submitted electronically via Patent Center with the filing of the specification.

### FIELD OF THE DISCLOSURE

The present disclosure relates to methods for the ex vivo expansion of hematopoietic stem cells and/or hematopoietic progenitor cells (HSPCs) and the differentiation of the expanded HSPCs ex vivo to form a cell product comprising functional monocytes and macrophages. The HSPCs can be CD34⁺ cells which are derived from one or more human subjects. In particular, the CD34⁺ cells can be derived from human cord blood and/or placental blood. The monocyte/macrophage cell product generated by the disclosed ex vivo methods from pooled unmatched, matched, or partially mismatched, cord blood and/or placental blood CD34⁺ stem and progenitor cells, comprises ~70% functional CD11b⁺ HLA-DR⁺ monocytes and macrophages and ~30% other myeloid cells, including granulocytes and dendritic cells. These monocytes and/or macrophage cell products are also comprised of about 70% CD14 CD16 double positive cells. The monocytes and macrophages that comprise the cell product demonstrate functional phagocytic capacity in vitro. The cell product produced by the disclosed methods can be used as a therapeutic agent against tumor cells, including both hematologic and solid tumors. This product can also be used as an antimicrobial therapeutic and as a therapeutic in repair of injured tissue. The produced cell product can also be used for autoimmune indications. Its specificity and/or potency can also be improved through, for example, genetically engineering the cells to express, for example, a receptor (such as a chimeric antigen receptor (CAR), a T cell receptor (TCR), a chimeric receptor, or other receptor), an immune cell engager, an antibody, a nanobody, a transcription factor, a cytokine, and/or a chemokine. In other embodiments, the function of the cell product can be improved by pre-loading the cells with an oncolytic virus or other immunomodulatory molecule for delivery to a solid tumor.

### BACKGROUND

Macrophages constitute a heterogeneous cell population representing innate immunity. Discovered at the end of 19th century by Ilya Mechnikov, macrophages have been identified in all tissues. Their chief competences are phagocytic activity and antigen presentation. Macrophages continuously monitor their microenvironments for the presence of pathogens, unfit cells, debris, and toxic metabolites, and release a variety of active substances including growth factors and cytokines. Human macrophages express several markers including CD14, CD16, CD68, CD163, CD11b, CD86, and CD206. According to the traditional concept, macrophages are classified into pro-inflammatory (M1), non-activated (M0) or anti-inflammatory (M2) subsets that play distinct roles in the initiation and resolution of inflammation.

Macrophages are cells involved in fundamental biological processes, including inflammation development and homeostasis support. They mediate host protection by engulfing and eliminating pathogens, by secreting a wide range of proinflammatory mediators that attract and activate immune cells at the site of infection, and by processing and presenting antigens to T lymphocytes, which propagates an adaptive immune response in the tissues.

Macrophages are also able to limit inflammation and mediate tissue repair and wound healing, largely by secreting anti-inflammatory and tissue remodeling factors and by phagocytizing apoptotic and necrotic cells. The foundation for the various and often opposite activities is formed by macrophages' capacity to sense the microenvironment and finetune their transcriptomic and functional programs according to homeostatic requirements. Dysregulation of these processes underlies many diseases. An exacerbated inflammatory response and/or impaired phagocytic/clearance activities of macrophages have been implicated in the pathogenesis of autoimmune, chronic inflammatory, cardiovascular, metabolic, neurodegenerative, infectious, and several hereditary diseases. In turn, insufficient inflammatory potential and/or excessive secretion of anti-inflammatory and tissue remodeling mediators induce fibrosis and promote cancer initiation, invasion, and metastasis.

Adoptive cell therapy (ACT) represents a promising approach to support standard cancer treatment strategies, including surgery, radiation, and chemotherapy. Efforts to strengthen the patient's immune system through administration of cellular immune therapeutics have resulted in significant improvements for cancer patients. In particular, the chimeric antigen receptor (CAR) T cell therapy has recently revolutionized the fight against hematologic malignancies. CARs are artificial receptors that consist of an extracellular antigen-binding unit, e.g., derived from a single chain antibody fragment (scFv), a hinge region, a transmembrane sequence, and an intracellular signaling domain responsible for specific cell activation. CAR principles were also applied to other immune cell types, such as natural killer (NK) cells, which showed encouraging results in first clinical trials in patients with CD19⁺ lymphoid tumors. However, successful use of CAR T and CAR NK against solid tumors remains to be fully established, and strategies to improve the efficacy and persistence of CAR T and CAR NK are currently being explored.

Another immune effector cell type potentially suitable for CAR cell therapy is macrophages since they have a high phagocytic capacity and are antigen-presenting cells. Macrophages can infiltrate solid tumors, actively attack cancer cells, and simultaneously orchestrate important immune responses and activate bystander cells to enhance the anti-tumor response. Furthermore, they are capable of cytokine secretion and exhibit a high loading capacity, which can be exploited for use of these cells as cargo vehicles. Macrophages were used as adoptive cell therapies against solid tumors and autologous transfer of blood-derived monocytes was shown to be safe.

Various methods have been described for producing macrophage cells ex vivo, such as generation from induced pluripotent stem cells, from isolated peripheral blood mononuclear cells, from peripheral blood stem or progenitor cells, or from umbilical cord blood stem or progenitor cells.

Macrophages can be used in vivo as a form of cell therapy, either unmodified or genetically modified to express a receptor (such as a CAR, a TCR, a chimeric receptor, or other receptor), an immune cell engager, an antibody, a nanobody, a transcription factor, a cytokine, and/or a chemokine to treat various cancer types, or infectious, inflammatory, and/or autoimmune diseases.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure provides methods for preparing compositions and preparation comprising monocytes and/or macrophage for use in immunotherapy. In particular, the present disclosure provides a method of preparing a monocyte and/or macrophage preparation for use in immunotherapy, comprising: selecting a plurality of umbilical cord blood or placental blood cells; preparing enriched CD34+ hematopoietic stem and progenitor cells (HSPCs) that are depleted of red blood cells and T cells; culturing the CD34+ enriched HSPCs in an expansion culture medium comprising interleukin-3 (IL-3), interleukin-6 (IL-6), thrombopoietin (TPO), Flt-3 ligand (Flt-3L), and stem cell factor (SCF) on a solid phase for a sufficient time to produce expanded HSPCs, wherein the expanded HSPCs do not substantially differentiate into CD11b+ HLA-DR+ cells during the expansion; and culturing the expanded HSPCs in an expansion and monocyte differentiation culture medium comprising effective amounts of Flt-3 ligand (Flt-3L), interleukin 3 (IL-3), and monocyte colony stimulating factor (M-CSF) and/or granulocyte-macrophage colony stimulating factor (GM-CSF) on a solid phase for a sufficient time to produce expanded HSPCs and monocytes, the cell composition comprising an average of about 40 to about 80% HLA-DR+ CD11b+ cells and an average of about 20 to about 60% other myeloid cells, the cell population also comprising about 70% CD14+ CD16+ cells; culturing the expanded HSPCs and monocytes in a macrophage differentiation culture medium comprising effective amounts of granulocyte-macrophage colony stimulating factor or M-CSF and serum or a non-animal sourced serum replacement for a sufficient time to produce a macrophage cell composition and/or preparation comprising about 60 to about 90% HLA-DR+ CD11b+ cells and an average of about 10 to about 40% other myeloid cells, the cell population also comprising about 7% CD14+ CD16− cells, about 66% CD14+ CD16+ cells, and about 5% CD14− CD16+ cells; and wherein the macrophages can be activated by pathogens or tumor cells.

The umbilical cord blood or placental blood cells used in the method can be from a non-HLA matched, matched, or partially mismatched donor or can be from umbilical cord blood or placental blood cells pooled from non-HLA matched, matched, or partially mismatched donors.

The expansion culture step of the method can further comprise a Notch ligand and fibronectin and the expansion culture can be carried out for about 3 to about 21 days.

In one embodiment of the disclosed method, the expansion and monocyte differentiation culture phases are carried out for about 7 to about 14 days. In another embodiment, the macrophage differentiation phase is carried out for about 6 to 8 days.

In certain embodiments of the method, the non-animal serum replacement is human AB serum, human serum albumin, or human platelet lysate. In certain embodiments, the HSPCs are not derived from somatic cells, embryonic stem cells, peripheral blood mononuclear cells or induced pluripotent stem cells. In certain embodiments of the method, the Notch ligand is DXI or an antibody specific for Notch.

In certain embodiments of the disclosed method, cells of the monocyte and/or macrophage composition and/or preparation are genetically modified. In certain embodiments of the method where the monocytes and/or macrophage are genetically modified, the genetic modification is during the expansion phase or subsequent to differentiation of the HSPCs, monocytes and/or macrophage. In certain embodiments of the method, the monocytes and/or macrophages of the composition and/or preparation are genetically modified to affect a gene knockdown, knockout, or knock-in. In those embodiments of the method, the gene targeted for knockdown or knockout is B2M, CIITA, a cytokine, a cytokine receptor, a chemokine, a chemokine receptor, a transcription factor, a growth factor receptor, an immunomodulatory molecule, an immune stimulatory molecule, an immune costimulatory molecule, and/or an immune costimulatory ligand. Where the gene is targeted for knock-in, the gene targeted is HLA-E, HLA-E and B2M, a cytokine, a cytokine receptor, a chemokine, a chemokine receptor, a transcription factor, a growth factor receptor, an immunoregulatory molecule, an immune stimulatory molecule, an immune costimulatory molecule, and/or an immune costimulatory ligand.

In certain embodiments of the disclosed method, the cells of the monocyte and/or macrophage composition and/or preparation are genetically modified to express an RNA, an enzyme, a receptor, a chimeric receptor, an immune cell engager, an antibody, a nanobody, a transcription factor, a cytokine and/or a chemokine. In these embodiments, the RNA is a shRNA, a siRNA or a gRNA.

In certain embodiments of the disclosed method, the receptor is a CAR or a TCR. In certain embodiments of the disclosed method, the receptor, chimeric receptor, immune cell engager, antibody, or nanobody specifically bind to a viral antigen, a bacterial antigen, a tumor-specific, a tumor-associated, or stroma antigen. The viral antigen can be present in a Cytomegalovirus (CMV), an Epstein Barr Virus (EBV), a Human Immunodeficiency Virus (HIV), a Herpes simplex virus (HSV), a Hepatitis virus, a Zika virus, an influenza virus, or a coronavirus. The Herpes virus can be HSV 1 or HSV 2, the Hepatitis virus is Hepatitis A, B, or C, and the coronavirus is SARS-CoV or SARS-CoV-2.

In certain embodiments of the disclosed method, the tumor-specific antigen, tumor-associated antigen, or stroma antigen is AFP, ALPP, ALPP2, ANTXR1, alpha-V beta-3 integrin, alpha-V beta-6 integrin, AXL, BCMA, B7-H3 (CD276), B7-H4 (VTCN1), carbonic anhydrase IX (CA1X), carcinoembryonic antigen (CEA), CD5, CD8, CD7, CD10, CD19, CD20, CD22, CD30, CD33, CD34, CD38, CD41, CD44, CD44V6, CD47, CD49c, CD49e, CD49f, CD56, CD61, CD66c, CD70, CD72, CD73, CD74, CD80, CD86, CD104,CD123, CD126, CD133, CD138, CD142, CD147, CD318, an antigen of a cytomegalovirus (CMV) infected cell (e.g., a cell surface antigen), cutaneous lymphocyte-associated antigen (CLA; a specialized glycoform of P-selectin glycoprotein ligand-1 (PSGL-1)), a chlorotoxin ligand, claudin 6 (CLDN6) claudin 18.2 (CLDN18.2), CLL1 CRLF2, DLL-3, DR4, DR5, EGF1R, epidermal growth factor receptor (EGFR), EGFR806, EGFRvIII, epithelial glycoprotein-2 (EGP-2), epithelial glycoprotein-40 (EGP-40), epithelial cell adhesion molecule (EpCAM), EpHA2, receptor tyrosine-protein kinases erb-B2,3,4 (erb-B2,3,4), ERBB, ERBB2, folate-binding protein (FBP), fetal acetylcholine receptor (AChR), FAP, folate receptor-alpha (FOLR1), FZD1, FZD2, FZD3, FZD4, FZD5, FZD6, FZD7, FZD8, FZD9, FZD10, Ganglioside G2 (GD2), Ganglioside G3 (GD3), GFRA4, GP100, GPC2, GPC3, GSPG4, GUCY2C, human Epidermal Growth Factor Receptor 2 (HER2), human telomerase reverse transcriptase (hTERT), ICAM1 (CD54), Interleukin-13 receptor subunit alpha-2 (IL-13Ralpha2), kappa-light chain, kinase insert domain receptor (KDR), KLK2, Lewis Y (LeY), L1 cell adhesion molecule (L1CAM; CD171), LMP1, LRRC15, melanoma antigen family A, 1 (MAGE-A1), MAGEA3, MAGEA4, MART1, mesothelin (MSLN), MET (c-Met; HGFR), MG7, mucin 1 (MUC1), TnMUC1, MUC3A, mucin 16 (MUC16), NECTIN4, NKG2D, an NKG2D ligand (for example, MIC-A, MIC-B, ULBP1, ULBP2, ULBP3, ULBP4, ULBP5, ULBP6), cancer-testis antigen NY-ESO-1, oncofetal antigen (h5T4), p53, PD-1, PD-L1, PD-L2, Proteinase3 (PR1), PRAME, prostate stem cell antigen (PSCA), prostate-specific membrane antigen (PSMA), REG3A (PAP), ROR1, ROR2, Survivin, Tyrosinase, tumor-associated glycoprotein 72 (TAG-72), TROP2, tetraspanin 8 (TSPAN8), vascular endothelial growth factor R2 (VEGF-R2), or Wilms tumor protein (WT-1). In certain particular embodiments, the tumor-specific or tumor-associated antigen is EGFR or any variant thereof, a NKG2D ligand, HER2, B7-H3, PSMA, PSCA, MUC1 or a variant thereof, mesothelin, or CEA.

In certain embodiments of the disclosed method, the CAR comprises an extracellular antigen binding domain, a hinge region, a transmembrane domain, and one or more intracellular signaling domains. The intracellular signaling domain can comprise one or more cytoplasmic or intracellular signaling domains of CD3zeta (CD3ζ), CD3delta, CD3epsilon, CD3gamma, CD4, CD8A, CD8B, CD2, CD7, LIGHT, CD27, CD28, 4-1BB (CD137), CD226 (DNAM1), B24 (CD244), ICOS (CD278), CTLA-4, GITR, OX40 (CD134), LAT, PD-1, TIM3, TIGIT, PD-L1, PD-L2, OX40L, 4-1BBL, ICOSLG, CD30L, CD30, CD36, CD68, CD40, CD70, CD80, CD83, CD86, CD163, CD204 (MSR1), CD206 (MRC1), CD209 (DC-SIGN), AGER (RAGE), CD276 (B7-H3), CD147, LILRB1, LILRB2, LILRB3, LILRB4, LILRB5, LILRA1, LILRA2, LILRA3, LILRA4, LILRA5, LILRA6, CLEC1A (CLEC1), CLEC1B (CLEC2), CLEC2A, CLEC2B, CLEC4D (DECTIN-3), CLEC4E (MINCLE), CLEC5A, CLEC6A (DECTIN-2), CLEC7A (DECTIN-1), CLEC8A (LOX-1)., CLEC9A (DNGR-1), CLEC10A (MGL), CLEC12A (MICL), SIGLEC1-11, SIGLEC14-16, AXL, MERTK, TYRO3, TREM2, CD11A (LFA-1; ITGAL), CD11B (ITGAM), CD11C (ITGAX), CSFIR (M-CSFR; CD115), GM-CSFR (CD116), CD14, CD16A (FcγRIIIa), CD32A (FcγRIIa), CD32B (FcγRIIb), CD32C (FcγRIIc), CD64 (FcγRI), FcRγ (FcεRIγ), MEGF10, CD79A, CD79B, CD19, TLR-1, TLR-2, TLR-3, TLR-4, TLR-5, TLR-6, TLR-7, TLR-8, TLR-9, MYD88, MAL, TRAM (TICAM2), TRIF (TICAM1), DAP-10, DAP-12, MAVS, STING, RIG-I, AIM2, NOD1-5, NLRP1-14, RIPK2, CASP1-10, CASP12-L, CASP12-S, CASP-14, a cytokine receptor, IFNGR, IFNGR1, IFNGR2, IFNAR, IFNAR1, IFNAR2, IFNLR1, CD116 (GM-CSFR), CSF2RA, CSF2RB, CSF1R (CD115; M-CSFR), IL10R, IL10RA, IL10RB, TGFBR, TGFBR1, TGFBR2. TNFRSF1A, TNFRSF1B), a chemokine receptor comprising CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, XCR1, CX3CR1, or any combination thereof; a transmembrane domain comprising a transmembrane domain of an intracellular signaling domain, and optionally comprises CD8, CD28, CD3zeta, CD4, 4-1BB, OX40, ICOS, or NKG2D; and a spacer region comprising a hinge region of IgG1, the CH₂CH₃ region of an immunoglobulin, a portion of CD3, a portion of CD28, or a portion of CD8.

In certain embodiments of the disclosed method, the immune cell engager specifically binds to one or more T cell surface proteins, one or more NK cell surface proteins, or one or more monocyte and/or macrophage surface protein. In certain embodiments of the disclosed method, the T cell surface protein preferably is CD3, CD28, and/or 4-1BB. In certain embodiments of the disclosed method, the NK cell surface protein is CD56, CD16, and/or NKG2D. In certain embodiments of the disclosed method, the monocyte and/or macrophage surface protein is CD64, CD40, CD80, and/or CD86.

In certain embodiments of the disclosed method, the transcription factor is a C/EBP transcription factor, a NF-κB transcription factor, a STAT transcription factor, a KLF transcription factor, a PPAR transcription factor, an AP-1 transcription factor, a NFAT transcription factor, a GATA transcription factor, a CREB transcription factor, or an IRF transcription factor.

In certain embodiments of the disclosed method, the cytokine or chemokine is an Interleukin, Interferon α, Interferon β, Interferon γ, Interferon λ, TGF β, TNF α, TNF β, GM-CSF, M-CSF, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, XCL1, XCL2, CX3CL1, CXCL1, CXCL2, CXLC3, CXCL4, CXCL5, CXCL6, CXCL7, CXCL8, CXCL9, CXCL10, CXCL11, CXCL12, CXCL13, CXCL14, CXCL16, or CXCL17. The interleukins of these embodiments of the method can be IL-1 α, IL-1β, IL-2, IL-4, IL-5, IL-6, IL-7, IL-8, IL-9, IL-10, IL-11, IL-12, IL-13, IL-15, IL-17, IL-18, IL-21, or IL-22.

In certain embodiments of the disclosed method, the monocyte and/or macrophage composition and/or preparation further comprises a cryoprotective agent. In certain embodiments of the disclosed method, the monocyte and/or macrophage composition and/or preparation is formulated to form a monocyte and/or macrophage formulation for infusion into a subject.

The present disclosure also provides a composition comprising the monocyte and/or macrophage composition and/or preparation produced by any of the methods above for use in immunotherapy. Immunotherapy as used herein comprises use of the described compositions and/or preparations as a therapeutic agent against tumor cells, use as an antimicrobial agent, use for autoimmune indications, or use as a therapeutic in repair of injured tissue.

In certain embodiments described in the present disclosure, the composition comprises the monocyte and/or macrophage composition and/or preparation produced by any of the methods described herein for use in delivery of small molecules, plasmid DNA, oncolytic virus, or other therapeutics. The present disclosure also provides compositions comprising the monocyte and/or macrophage composition and/or preparation produced by any of the methods described herein for use in combination with other therapeutic compositions comprising unmodified or genetically modified T cells or NK cell therapies, antibody or nanobody therapeutics, immune cell engagers, cytokines, chemokines, and/or oncolytic virus.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A and 1B depict the ex vivo expansion of pooled cord blood CD34⁺ cells. FIG. 1A shows approximately 78,000 cells were generated from each CD34⁺ cell seeded into culture on day 0. FIG. 1B shows the high viability of the cells throughout the culture process. n=2; mean±s.d.

FIGS. 2A and 2B depict the ex vivo differentiation and expansion of monocytes and macrophages from pooled cord blood CD34⁺ cells. FIG. 2A shows the significant differentiation of cells toward becoming HLA-DR⁺ CD11b⁺ occurs during Phase 2 and Phase 3 of culture. FIG. 2B shows the total fold expansion of approximately 54,000 HLA-DR⁺ CD11b⁺ cells from each starting CD34⁺ cell seeded into culture on day 0. N=2; mean±s.d.

FIGS. 3A through 3C show the HLA-DR and CD11b co-expression increases significantly during monocyte differentiation (end of Phase 2; FIG. 3B) and is maintained during macrophage differentiation (end of Phase 3, FIG. 3C) in the presence of either fetal bovine serum (FBS) or human platelet lysate (HPL).

FIGS. 4A through 4D demonstrate that HLA-DR⁺ CD11b⁺ macrophages generated ex vivo express markers associated with M1 (FIGS. 4A and 4B) or M2 (FIGS. 4C and 4D) polarization when differentiated in either GM-CSF or M-CSF, respectively, during Phase 3 of culture.

FIG. 5 demonstrates that ex vivo generated macrophages effectively phagocytose pHrodo™ Staphylococcus aureus BioParticles™ in an in vitro phagocytosis assay. Bioparticles become fluorescent within the acidic environment of the macrophage cytoplasmic phagosomes.

FIG. 6 depicts macrophage generated ex vivo using either GM-CSF or M-CSF during Phase 3 of culture can be cryopreserved with high levels of post-thaw cell recovery and viability.

FIG. 7 depicts an in vitro phagocytosis assay using fluorescent pHrodo™ S. aureus BioParticles™. Monocytes harvested and cryopreserved at the end of Phase 2 of culture or macrophage harvested and cryopreserved at the end of Phase 3 of culture each demonstrate phagocytosis following cryopreservation and thaw.

FIG. 8 shows the cell surface expression of CD19 CAR on monocytes and macrophages generated by ex vivo expansion and differentiation of CD34⁺ HSPCs transduced with a CD19 CAR viral vector.

FIGS. 9A though 9C demonstrate that engineering monocytes and macrophages to express a CD19 CAR does not reduce their total fold expansion (FIG. 9A), cell viability (FIG. 9B), or phagocytic capacity (FIG. 9C) compared with control un-transduced monocytes and macrophages.

### DETAILED DESCRIPTION

The present disclosure provides herein a culture method for the ex vivo expansion of hematopoietic stem and/or hematopoietic progenitor cells (HSPCs) followed by the ex vivo differentiation of the expanded HSPCs to functional monocytes and macrophages. The culture method comprises three phases: (1) HSPC Expansion, comprised of culturing HSPCs with serum-free expansion medium in the presence of cytokines and growth factors, with or without activation of the Notch receptor signaling pathway by Delta1^{extIgG} (DXI) binding, to induce proliferation of stem and progenitor cells without terminal differentiation; (2) Expansion and Monocyte Differentiation, comprised of culturing expanded HSPCs with serum-free medium in the presence of cytokines and growth factors, with or without activation of the Notch receptor signaling pathway by DXI binding, to induce further expansion and differentiation of cells to a monocyte phenotype; and (3) Macrophage Differentiation, comprised of culturing differentiated monocytes with medium containing serum or a serum replacement and growth factors to induce differentiation of monocytes to a macrophage phenotype. Typically, the hematopoietic stem cells, or stem and progenitor cells, are CD34⁺. In some embodiments, the hematopoietic stem cells, or hematopoietic stem and progenitor cells, are derived from human umbilical cord blood and/or human placental blood. Use of cord blood and/or placental blood derived CD34⁺ cells as the starting material, compared with adult-derived cells, allows for the generation of monocytes and macrophages that are not terminally differentiated or exhausted and are likely to have greater capacity for proliferation and persistence than adult monocytes derived from peripheral blood.

### Preparation of a CD34⁺ Enriched Cell Population

The CD34⁺ enriched cell population comprises hematopoietic stem or hematopoietic stem and progenitor cells and has been substantially depleted of T cells and red blood cells, therefore leaving enriched numbers of CD34⁺ hematopoietic stem or hematopoietic stem and progenitor cells. The hematopoietic stem or hematopoietic stem and progenitor cells can in some embodiments comprise multiple HLA-types because the hematopoietic stem or hematopoietic stem and progenitor cells are not matched to each other prior to pooling, and also are not matched to the patient. In other embodiments, the HSPCs are either HLA matched or partially mismatched with the subject. As used herein, substantially depleted of T cells refers to less than 1% CD3⁺ cells, or less than 0.5% CD3⁺ cells, or less than 0.1% CD3⁺ cells, in the enriched CD34⁺ cell population.

In some embodiments, the CD34⁺ hematopoietic stem cells or hematopoietic stem and progenitor cells are derived from cord blood or from placental blood. Human umbilical cord blood and/or human placental blood are typical sources of the cord blood stem cells. Such blood can be obtained by methods known in the art. See, e.g., U.S. Pat. Nos. 5,004,681 and 7,147,626 and U.S. Patent Publication No. 2013/0095079, incorporated herein by reference, for a discussion of collecting cord and placental blood at the birth of a human. Umbilical cord blood and/or human placental blood collections are made under sterile conditions. Upon collection, cord or placental blood is mixed with an anticoagulant, such as CPD (citrate-phosphate-dextrose), ACD (acid citrate-dextrose), Alsever's solution (Alsever et al., 1941, N. Y. St. J. Med. 41:126), De Gowin's Solution (De Gowin, et al., 1940, J. Am. Med. Ass. 114:850), Edglugate-Mg (Smith, et al., 1959, J. Thorac. Cardiovasc. Surg. 38:573), Rous-Turner Solution (Rous and Turner, 1916, J. Exp. Med. 23:219), other glucose mixtures, heparin, ethyl biscoumacetate, and the like. See, generally, Hurn, 1968, Storage of Blood, Academic Press, New York, pp. 26-160). In one embodiment, ACD can be used.

Cord blood can preferably be obtained by direct drainage from the umbilical cord and/or by needle aspiration from the delivered placenta at the root and at distended veins. Preferably, the collected human cord blood and/or placental blood is free of contamination and, in particular, viral contamination.

In certain embodiments, the following tests can be performed on the collected blood, either routinely or where clinically indicated:

Bacterial culture: To ensure the absence of microbial contamination, established assays can be performed, such as routine hospital cultures for bacteria under aerobic and anaerobic conditions.

Diagnostic screening for pathogenic microorganisms: To ensure the absence of specific pathogenic microorganisms, various diagnostic tests can be employed. Diagnostic screening for any of the numerous pathogens transmissible through blood can be done by standard procedures. As one example, the collected blood sample (or a maternal blood sample) can be subjected to diagnostic screening for the presence of viruses. Any of numerous known assay systems can be used, based on the detection of virions, viral-encoded proteins, virus-specific nucleic acids, antibodies to viral proteins, and the like. The collected blood can also be tested for infectious diseases, including but not limited to Human Immunodeficiency Virus-1 or 2 (HIV-1 or HIV-2), human T-Cell lymphotropic virus I and II (HTLV-I and HTLV-II), Hepatitis B, Hepatitis C, Cytomegalovirus, Syphilis, corona virus, West Nile Virus, and the like.

Preferably, prior to collection of the cord blood, a maternal health history is determined to identify risks that the cord blood cells might pose, e.g., transmitting genetic or infectious diseases, such as cancer, leukemia, immune disorders, neurological disorders, hepatitis, or AIDS. The collected cord blood can have undergone testing for one or more of cell viability, HLA typing, ABO/Rh typing, CD34⁺ cell count, and total nucleated cell count.

Once the umbilical cord blood and/or placental blood is collected from human donors at birth, the blood is processed to produce an enriched hematopoietic stem cell population, or an enriched hematopoietic stem and progenitor cell population. Preferably, the hematopoietic stem cells, or hematopoietic stem and progenitor cells, are CD34⁺ cells or predominantly CD34⁺ cells. Preferably, the hematopoietic stem cell or hematopoietic stem and progenitor cell population is substantially depleted of T cells and of red blood cells, resulting in a cell population enriched for CD34⁺ stem cells and/or CD34⁺ stem and progenitor cells. Enrichment thus refers to a process wherein the percentage of hematopoietic stem cells, or hematopoietic stem and progenitor cells, in the cell population is increased (relative to the percentage in the population before the enrichment procedure). Purification is one example of enrichment. Typically, a starting cord blood unit is made up of ~0.25%-0.75% CD34⁺ cells, and the enriched selected cell population is made up of ~92%-99% CD34⁺ cells.

Prior to processing for enrichment, the collected cord and/or placental blood can be fresh or have been previously cryopreserved. Any suitable technique known in the art for cell separation/selection can be used to carry out the enrichment for hematopoietic stem cells, or hematopoietic stem and progenitor cells. Methods which rely on differential expression of cell surface markers can be used. For example, cells expressing the cell surface marker CD34 can be positively selected using a monoclonal antibody to CD34, such that cells expressing CD34 are retained, and cells not expressing CD34 are not retained. Moreover, the separation techniques employed should maximize the viability of the cell population to be selected. The particular technique employed will depend upon the efficiency of separation, cytotoxicity of the methodology, ease and speed of performance, and the necessity for sophisticated equipment and/or technical skill.

Procedures for separation can include magnetic separation, using antibody-coated magnetic beads, affinity chromatography, and “panning” with antibody attached to a solid matrix, e.g., plate, or other convenient technique. Techniques providing accurate separation/selection include fluorescence activated cell sorters, which can have varying degrees of sophistication, e.g., a plurality of color channels, low angle and obtuse light scattering detecting channels, impedance channels, and the like.

The antibodies used in the selection process may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the remaining cells. Examples include, for example, the FDA approved CliniMACs® processing system (Miltenyi Biotec B.V. & Co. KG), the Dynabeads™ CD34 isolation system (Invtrogen Inc.), the EasySep™ Human CD34 Positive Selection Kit (Stemcell Technologies, Inc.), and the like.

In a preferred embodiment, fresh cord blood units are processed to select for, i.e., enrich for, CD34⁺ cells using anti-CD34 antibodies directly or indirectly conjugated to magnetic particles in connection with a magnetic cell separator, for example, the CliniMACS® Cell Separation System (Miltenyi Biotec, Bergisch Gladbach, Germany), which employs nano-sized super-paramagnetic particles composed of iron oxide and dextran coupled to specific monoclonal antibodies. The CliniMACS® Cell Separator is a closed sterile system, outfitted with a single-use disposable tubing set. The disposable tubing set can be used for and discarded after processing a single unit of collected cord and/or placental blood to enrich for CD34⁺ cells.

In an embodiment, two or more umbilical cord blood and/or placental blood units can be pooled prior to enriching for the hematopoietic stem cells, or hematopoietic stem and progenitor cells. In another embodiment, individual populations of CD34⁺ stem cells or CD34⁺ stem and progenitor cells can be pooled after enriching for the hematopoietic stem cells, or hematopoietic stem and progenitor cells. In specific embodiments, the number of umbilical cord blood and/or placental blood units, or populations of hematopoietic stem or hematopoietic stem and progenitor cells that are pooled is 2, 3, 4, 5, 6, 7, 8, 9, 10, 15, 20, 25, or at least any of the foregoing numbers. In some embodiments, the pool contains 2 to 8, 2 to 10, 4 to 8, 4 to 10, 2 to 20, 4 to 20, 2 to 25 or 4 to 25, and no more than 20 or 25, umbilical cord blood and/or placental blood units, or CD34⁺ hematopoietic stem or hematopoietic stem and progenitor cell populations. The umbilical cord blood and/or placental blood units or hematopoietic stem or hematopoietic stem and progenitor cell populations are pooled without regard to the HLA-type of the hematopoietic stem or hematopoietic stem and progenitor cells. In certain embodiments, the cells in the pool are derived from the umbilical cord blood and/or placental blood of individuals of the same race, e.g., African-American, Caucasian, Asian, Hispanic, Native-American, Australian Aboriginal, Inuit, Pacific Islander, or derived from umbilical cord blood and/or placental blood of individuals of the same ethnicity, e.g., Irish, Italian, Indian, Japanese, Chinese, Russian, and the like. In other embodiments, the cells in the pool are combined without regard to race or ethnicity.

Optionally, prior to enrichment for hematopoietic stem cells or hematopoietic stem and progenitor cells, the red blood cells and white blood cells of the cord blood or placental blood can be separated. Once the separation of the red blood cells and the white blood cells has taken place, the red blood cell fraction can be discarded, and the white blood cell fraction can be processed in the magnetic cell separator as described above to enrich for CD34⁺ hematopoietic stem cells or hematopoietic stem and progenitor cells. Separation of the white and red blood cell fractions can be performed by any method known in the art, including centrifugation techniques. Other separation methods that can be used include, for example, the use of commercially available products FICOLL™ or FICOLL-PAQUE™ or PERCOLL™ (GE Healthcare, Piscataway, New Jersey). FICOLL-PAQUE™ is normally placed at the bottom of a conical tube, and the whole blood is layered above. After being centrifuged, the following layers will be visible in the conical tube, from top to bottom: plasma and other constituents, a layer of mono-nuclear cells called buffy coat containing the peripheral blood mononuclear cells (white blood cells), FICOLL-PAQUE™, and erythrocytes and granulocytes, which should be present in pellet form. This separation technique allows easy harvest of the peripheral blood mononuclear cells (PBMCs).

Optionally, prior to CD34⁺ cell selection, an aliquot of the cord blood or placental unit can be checked for total nucleated cell count and/or CD34⁺ cell content. In a specific embodiment, after the CD34⁺ cell selection, both CD34⁺ and CD34⁻ cell fractions are recovered. Optionally, DNA can be extracted from a sample of the CD34⁻ cell fraction for initial HLA typing and future chimerism studies, even though HLA matching of the CD34⁺ cell fraction to the patient or to the other cord blood or placental blood units is not done.

### Phase 1—Expansion of Hematopoietic Stem and Progenitor Cells (HSPCs)

“Expanded HSPCs” refers to hematopoietic stem cells or stem and progenitor cells that have been subjected to a technique for expanding the hematopoietic stem cells, or hematopoietic stem and progenitor cells ex vivo, which technique has been shown to result in (i) an increase in the number of hematopoietic stem cells, or hematopoietic stem and progenitor cells, in an aliquot of the cells thus expanded, or (ii) an increased number of severe combined immunodeficiency (SCID) repopulating cells determined by limiting-dilution analysis as shown by enhanced engraftment in non-obese diabetic (NOD)/SCID mice infused with an aliquot of the cells thus expanded. These are relative to that seen with an aliquot of the cells not subjected to the expansion technique. (See, for example, US Patent Application Publication No. 2013/0095079; Delaney et al., Nat. Med. 16(2):232-236, 2010).

In a specific embodiment, the umbilical cord blood and/or placental blood units are red cell depleted, and the number of CD34⁺ cells in the red cell depleted fraction is determined. Preferably, the umbilical cord blood and/or placental blood samples enriched for CD34⁺ cells are seeded into tissue culture treated plastic culture vessels lacking any additional coating at a density of 9,000 to 10,000 cells/cm².

After the hematopoietic stem cells or hematopoietic stem and progenitor cells have been isolated (e.g., from human cord blood and/or human placental blood collected from humans at birth) according to the enrichment methods described above or other methods known in the art, the hematopoietic stem cells or hematopoietic stem and progenitor cells are expanded to increase the number of hematopoietic stem cells or hematopoietic stem and progenitor cells, e.g., CD34⁺ cells. Any method known in the art for expanding the number of hematopoietic stem cells or hematopoietic stem and progenitor cells that gives rise to an expanded (i.e., increased number of) population of hematopoietic stem cells or hematopoietic stem and progenitor cells can be used. Preferably, the hematopoietic stem cells or hematopoietic stem and progenitor cells are cultured under cell growth conditions (e.g., promoting mitosis) such that the hematopoietic stem cells or hematopoietic stem and progenitor cells grow and divide (proliferate) to obtain an expanded population of CD34⁺ hematopoietic stem cells or hematopoietic stem and progenitor cells. In one embodiment, individual populations of hematopoietic stem cells or hematopoietic stem and progenitor cells derived from an umbilical cord blood and/or placental blood of a single human at birth can be pooled, without matching to the HLA type of the other hematopoietic stem cells or hematopoietic stem and progenitor cells, prior to or after expansion. In another embodiment, the hematopoietic stem cells or hematopoietic stem and progenitor cells are expanded prior to pooling. Preferably, the technique used for expansion is one that has been shown to (i) result in an increase in the number of hematopoietic stem cells, or hematopoietic stem and progenitor cells, e.g., CD34⁺ cells, in the expanded stem cell product relative to the unexpanded population of hematopoietic stem cells or stem and progenitor cells, where the unexpanded cell population and expanded cell population are from different aliquots of the same source of stem or stem and progenitor cells, wherein the expanded cells but not the unexpanded cells are subjected to the expansion technique.

Expansion techniques include but are not limited to those described in U.S. Pat. No. 7,399,633 B2; U.S. Patent Publication No. 2013/0095079; Delaney et al., 2010, Nature Med. 16 (2): 232-236; Zhang et al., 2008, Blood 111:3415-3423; or Himburg et al., 2010, Nature Medicine 16 (4): 475-82, each incorporated herein by reference, as well as those described below.

In one embodiment, the hematopoietic stems cells or hematopoietic stem and progenitor cells are cultured in culture medium in the presence of growth factors, and are exposed to cell growth conditions (e.g., promoting mitosis) such that the hematopoietic stem or hematopoietic stem and progenitor cells proliferate to obtain an expanded population of hematopoietic stem or hematopoietic stem and progenitor cells. In a preferred embodiment, the hematopoietic stem or hematopoietic stem and progenitor cells are exposed to cell growth conditions (e.g., promoting mitosis) such that the hematopoietic stem or hematopoietic stem and progenitor cells proliferate to generate an expanded hematopoietic stem or hematopoietic stem and progenitor cell population. In certain embodiments, the increase in the number of CD34⁺ cells (or other suitable antigen-positive cells) as a percentage of cells in the expanded stem cell product, relative to the population prior to the enrichment procedure, is at least 25-, 50-, 75-, 100-, 150-, 200-, 250-, 300-, 350-, 400- or at least 350-fold, and preferably is 100- to 200-fold or 100- to 400-fold. The expanded hematopoietic stem or hematopoietic stem and progenitor cell population so obtained can be frozen and stored for later use.

In specific embodiments, the hematopoietic stem or hematopoietic stem and progenitor cells are cultured for 2, 3, 4, 5, 6, 7, 8, 9, 10, 11, 12, 13, 14, 15, 16, 17, 18, 19, 20, 21, 22, 23, 24, or 25 days or more; or, preferably, the hematopoietic stem or hematopoietic stem and progenitor cells are cultured for 3, 7, 10, 14, or 21 days. In more preferred embodiments, the cells are expanded for at least 10 days or from about 14 to about 21 days.

An exemplary culture condition for expanding the hematopoietic stem or hematopoietic stem and progenitor cells includes culturing the cells for about 14 to 21 days in the presence of a serum free medium supplemented with the following human growth factors: stem cell factor, Flt-3 receptor ligand, Thrombopoietin, Interleukin-6 and Interleukin-3. Preferably, the foregoing growth factors are present at the following concentrations: 50 to 300 ng/mL stem cell factor, 50 to 300 ng/mL Flt-3 receptor ligand, 50 to 100 ng/ml Thrombopoietin, 50 to 100 ng/ml Interleukin-6 and 10 ng/ml Interleukin-3. In more specific embodiments, 50 ng/mL stem cell factor, 50 ng/ml of Flt-3 receptor ligand, 50 ng/ml Thrombopoietin, 50 ng/ml Interleukin-6 and 10 ng/ml Interleukin-3, are used. In a more preferred embodiment, the cell culture medium consists of X-VIVO™-10 (Lonza). In alternative embodiments, the base medium used during HSPC expansion culture can be StemSpan™ SFEM II, StemPro™-34 SFM, X-VIVO™-15, PRIME-XV Expansion XSFM, CellGenix® SCGM, StemLine® or StemLine® II Hematopoietic Stem Cell Expansion Medium, or StemMACS HSC Expansion Media.

### Phase 2—Expansion and Monocyte Differentiation

### Monocyte and Macrophage Descriptions and Phenotypic Markers

Monocytes are approximately 0.10 μm diameter round mononuclear white blood cells that travel from bone marrow through the peripheral blood to tissues where they can further differentiate into macrophages or dendritic cells. As part of the innate immune system, stimulated monocytes demonstrate properties of chemotaxis and phagocytosis and can secrete cytokines that attract and/or activate additional cells of the innate and adaptive immune system. Cytokines released by monocytes include, but are not limited to, tumor necrosis factor alpha (TNFα), interleukin 6 (IL-6), interleukin 12 (IL-12), and interleukin 15 (IL-15).

Macrophages are large (approximately 21 μm diameter), complex, adherent, vacuolated white blood cells derived from monocytes, and are located mostly within tissues, where they can act as phagocytic scavengers to clear cellular debris related to cell aging or wound healing, can digest and present pathogen-related antigens to other immune cells to initiate an immune response, and can secrete powerful cytokines involved in inflammation and the regulation of immune responses. Tissue resident macrophages often perform functions specific to the tissue in which they reside but are generally classified into simplified groupings based on their broad functional and activation status, markers expressed on the cell surface, and cytokines released. The “classically activated” M1 polarized macrophages are pro-inflammatory, anti-pathogenic cells that express surface markers including, but not limited to, CD40, CD80, CD86, and interferon gamma (IFNγ) receptor, and can secrete cytokines including, but not limited to, TNFα, IL-12, and IL-6. The “alternatively activated” M2 polarized macrophages are anti-inflammatory and involved in tissue remodeling and immune regulation, express the surface markers including, but not limited to, CD163, CD206, CD209, and interleukin 4 (IL-4) receptor alpha, and can secrete cytokines including, but not limited to, interleukin 10 (IL-10) and transforming growth factor beta (TGFβ). Tumor-associated macrophages (TAMs) are identified within a tumor microenvironment and are often influenced by that microenvironment to adopt an immunosuppressive phenotype.

To further expand the cell population and to differentiate the expanded CD34⁺ cells produced in Phase 1 (the HSPC Expansion culture), cells were harvested and washed with, for example, phosphate buffered saline (PBS) by centrifugation, and resuspended in Phase 2 culture medium. The base cell culture medium for this phase can be selected from, for example, StemSpan™ SFEM II, StemPro™-34 SFM, X-VIVO™-10, X-VIVO™-15, or IMDM. In a preferred embodiment, the base cell culture medium is X-VIVO™-10 medium (Lonza).

During this phase the expanded HSPCs are also contacted with human growth factors that can continue to expand the population of HSPCs as in Phase 1, and also include human cytokines that induce the differentiation of the expanded HSPCs into Monocytes. The human growth factors and/or cytokines can include monocyte colony stimulating factor (M-CSF), granulocyte macrophage colony stimulating factor (GM-CSF), Flt3-L, IL-6 and/or IL-3. An exemplary culture condition for further expanding the hematopoietic stem or hematopoietic stem and progenitor cells and inducing the differentiation of the expanded HSPCs into monocytes includes culturing the cells for about 7, 10, or 14 days in the presence of a serum free medium supplemented with the following human growth factors: M-CSF (50 ng/mL), Flt-3 receptor ligand (Flt3-L; 50 ng/mL), IL-3 (10 ng/ml), and IL-6 (50 ng/ml). In an alternative embodiment, the Expansion and monocyte differentiation media supplements can include a combination of 50 ng/mL MCSF, 50 ng/mL GM-CSF, 50 ng/mL Flt3L, and 10 ng/ml IL-3.

The Monocyte cell product produced can be cryopreserved for later use or used directly in Phase 3—Macrophage Differentiation. The monocyte cell product can also be formulated as a pharmaceutical product.

### Phase 3—Macrophage Differentiation

At the end of the Phase 2 (Expansion and Monocyte Differentiation culture), cells were harvested and washed with PBS by centrifugation, and resuspended in Phase 3 culture medium supplemented with human growth factors and other factors that can induce to differentiation of monocytes into macrophage. Alternately, cells cryopreserved at the end of Phase 2 can be thawed and resuspended in Phase 3 culture medium. Phase 2 cells enriched for monocytes were seeded into tissue culture treated plastic culture vessels lacking any additional coating at a density of about 130,000 to 230,000 cells/cm². Cells were cultured for 6 to 8 days with a feed with Phase 3 medium about 3 to about 4 days into the culture period. Preferably the cells are cultured for about 7 days. At the end of Phase 3 culture, non-adherent cells are collected with the culture medium into a sterile conical tube. The remaining adherent cells are incubated to loosen them from the tissue culture substrate with, for example, TrypLE™ Express Enzyme (Gibco), followed by scraping of the tissue culture plastic surface, and the released adherent cells are combined with the collected non-adherent cells and washed by centrifugation. Washed cells can be used for in vitro or in vivo analyses, formulation into a pharmaceutical cell product, and/or cryopreserved.

In certain embodiments the base cell culture medium can be selected from X-VIVO™-10, X-VIVO™-15, RPMI 1640, IMDM, or ImmunoCult™-SF Macrophage Medium. In a specific preferred embodiment, the base cell culture medium is RPMI 1640.

The base cell culture medium is also typically supplemented with serum or a serum replacement. In certain embodiments the serum or serum replacement can be fetal bovine serum (FBS), human AB serum, human platelet lysate (HPL), and/or human serum albumin (HSA). In more specific embodiments, the serum or serum replacement is about 10% FBS; or about 10% human AB serum; or about 2.5% HPL, 5% HPL, or 10% HPL; or about 0.25% HSA, 0.5% HSA, 1% HSA, or 2% HSA.

The base medium is also supplemented with certain human growth factors and/or cytokines to induce the differentiation of the monocytes into macrophage. In certain embodiments the human growth factors and/or cytokines can be, for example, GM-CSF and/or M-CSF. In certain embodiments, about 10 ng/ml, 25 ng/ml, or 50 ng/ml of GM-CSF can be used. Or, in certain embodiments, about 25 ng/ml, 50 ng/mL, or 100 ng/ml M-CSF can be used. In a specific preferred embodiment, the base culture media is RPMI 1640, supplemented with 10 ng/mL GM-CSF and either 2.5% HPL or 10% FBS.

As above, the macrophage cell product produced at the end of Phase 3 can be cryopreserved using, for example, CryoStor® CS10 (Biolife Solutions) for later use or used directly to formulate a pharmaceutical product.

### Alternative Embodiments Wherein the Tissue Culture Substrate is Coated

In vitro culture of differentiating cells with feeder cell layers or with a tissue culture vessel substrate or coating can be done to activate cell signaling pathways that influence cell differentiation toward a specific desired lineage or activation status. During the Phase 1 HSPC Expansion culture, cells have been cultured in tissue culture treated plastic culture vessels without additional coating or pre-treatment. In certain embodiments, the enriched HSPCs have been cultured in a tissue culture substrate pre-coated with a Notch ligand and a portion of fibronectin as used in well-known methods for HSPC expansion. In particular embodiments, 0.1 μg/cm² Delta1^{extIgG} (DXI) and 0.8 μg/cm² recombinant human fibronectin fragment (RetroNectin®), 0.16 μg/cm² DXI and 0.8 μg/cm² RetroNectin®, 0.2 μg/cm² DXI and 0.8 μg/cm² RetroNectin®, or 0.4 μg/cm² DXI and 0.8 μg/cm² RetroNectin® were used to coat the tissue culture substrate prior to Phase 1 expansion of the HSPCs. These cultures were allowed to expand for all 7 days of a 7-day Phase 1 culture period, for the first 7 days of a 14- or 21-day Phase 1 culture period, and for the first 14 days of a 21-day Phase 1 culture period. In additional embodiments, cells were cultured in Phase 2 Expansion and Monocyte Differentiation culture, in tissue culture treated plastic culture vessels without additional coating or pre-treatment; pre-coated with 0.1 μg/cm² DXI and 0.8 μg/cm² RetroNectin®, with 0.2 μg/cm² DXI and 0.8 μg/cm² RetroNectin®, and with 0.4 μg/cm² DXI and 0.8 μg/cm² RetroNectin®. The cells were then cultured in Phase 3 Macrophage Differentiation in tissue culture treated plastic culture vessels without additional coating or pre-treatment. These methods all resulted in the production of a monocyte and/or macrophage product, although the yields were not better than those methods where Notch ligand was not used.

### Cell Engineering or Cell Modification

The monocytes/macrophage cell product generated through these methods can be genetically modified during any culture phase during their expansion or differentiation through standard methods of cell engineering that can include viral transduction with lentiviral, retroviral, adenoviral, or adeno-associated viral (AAV) vectors, or electroporation of mRNA, short hairpin RNA (shRNA), silencing RNA (siRNA), or guide RNA (gRNA), or plasmid DNA or covalently closed DNA or transposons or enzymes, or combinations of these materials, or lipid fusion with cell membranes to deliver mRNA, shRNA, siRNA, gRNA, or plasmid DNA or covalently closed DNA or transposons or enzymes, or combinations of these materials encapsulated within simple lipids or lipid nanoparticles. Depending upon the cell engineering method and material chosen, the genetic modification can be integrated within the genome of the cell and present through the lifespan of the cell, or the genetic modification may not be integrated within the genome of the cell and be only temporarily present within the cell.

Such cell engineering can be carried out for the purposes of prolonging the lifespan of the modified cells, enhancing endogenous functions of the modified cells, stabilizing desired phenotypes of the modified cells, introducing new capabilities or functions to the modified cells, or combinations of the above. Such modifications could include expression of a receptor (such as a CAR, a TCR, a chimeric receptor or other receptor), an immune cell engager, an antibody, a nanobody, a transcription factor, a cytokine, and/or a chemokine. Such modifications can also include knockdown, knockout, and/or knock-in of a gene or multiple genes. Another potential modification can include pre-loading macrophages with immune activating cargo, such as an oncolytic virus or other immunomodulatory molecule, for delivery to the otherwise immunosuppressive solid tumor microenvironment.

### Engineering for Gene Knockdown, Knockout, or Knock-in

In certain embodiments, the monocyte/macrophage product is engineered to have one or more gene knockdowns, knockouts, or knock-ins. In some embodiments, the gene knockdown is achieved by the use of shRNAs or siRNAs. In some embodiments the gene knockdown, knockout, or knock-in is achieved by targeted enzymatic cleavage followed by homology-directed repair or non-homologous end joining. In some embodiments, the enzyme can include but is not limited to Cas enzymes (Cas 7-11, Cas9, dCas9, nCas9, Cas12a), zinc-finger nucleases (ZFNs), or transcription activator-like effector nucleases (TALENs). In some embodiments, the enzyme is fused to one or more activating or repressor effector domains such as Krüppel-associated box (KRAB) domain, VP64, Rta, p65, or HSF1. In some embodiments, targeted enzymatic cleavage is achieved using nucleic acids such as guide RNAs (gRNAs). In some embodiments, gene knockdown, knockout, or knock-in is achieved through transient expression of enzymes and/or nucleic acid(s). In some embodiments, gene knockdown, knockout, or knock-in is achieved through sustained expression of enzymes and/or nucleic acid(s). In some embodiments, the enzymes and nucleic acid(s) are delivered via transfection, electroporation, or viral transduction. In some embodiments, plasmids or viral vectors encode the nucleic acid sequences required for shRNAs, siRNAs, gRNAs, enzymes, or the transgene for targeted gene knock-in. In certain embodiments, the gene targeted for knockdown or knockout includes, but is not limited to beta-2-microglobulin (B2M) or class II major histocompatibility complex transactivator (CIITA). In some embodiments, the gene targeted for knockdown or knockout includes a cytokine, a cytokine receptor, a chemokine, a chemokine receptor, a transcription factor, a growth factor receptor, an immunomodulatory molecule, a stimulatory molecule, a costimulatory molecule, and/or a costimulatory ligand. In some embodiments, the gene that is knocked-in encodes, for example, HLA-E or HLA-E and B2M. In some embodiments, the gene that is knocked-in encodes a cytokine, a cytokine receptor, a chemokine, a chemokine receptor, a transcription factor, a growth factor receptor, an immunomodulatory molecule, a stimulatory molecule, a costimulatory molecule, and/or a costimulatory ligand. In some embodiments, the transgene that is knocked-in is a CAR or Non-CAR construct as described below.

### Cell Engineering for Expression of Chimeric Antigen Receptors (CARs)

In some embodiments, the monocyte/macrophage product is genetically modified to express a CAR. In some embodiments, a CAR can comprise an extracellular antigen-binding domain, a spacer (or hinge) region, a transmembrane domain, and one or more intracellular signaling domains, wherein the extracellular antigen-binding domain specifically binds to an antigen, e.g., a tumor antigen or a pathogen antigen, including for example a viral or bacterial antigen.

Generations of CARs include the following. “First generation” CARs are typically composed of an extracellular antigen binding domain (e.g., a single-chain variable fragment (scFv)) fused to a transmembrane domain, fused to cytoplasmic/intracellular signaling domain of the T cell receptor chain. “First generation” CARs typically have the intracellular signaling domain from the CD3zeta-chain, which is the primary transmitter of signals from endogenous TCRs. “First generation” CARs can provide de novo antigen recognition and cause activation of cells through their CD3zeta chain signaling domain in a single fusion molecule, independent of HLA-mediated antigen presentation. “Second generation” CARs add intracellular signaling domains from various co-stimulatory molecules (e.g., CD28, 4-1BB, ICOS, OX40, or 2B4) to the cytoplasmic tail of the CAR to provide additional signals to the cell. “Second generation” CARs comprise those that provide both co-stimulation (e.g., CD28, 4-1BB or 2B4) and activation (CD3zeta). Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL). “Third generation” CARs comprise those that provide multiple co-stimulation (e.g., CD28 and 4-1BB) and activation (CD3zeta).

### Extracellular Antigen-Binding Domain of a CAR

In certain non-limiting embodiments, the extracellular antigen-binding domain of the CAR (embodied, for example, as an scFv or an analog thereof) binds to an antigen with a dissociation constant (K_{d}) of about 2×10⁻⁷ M or less. In certain embodiments, the K_{d} is about 1×10⁻⁷ M or less, about 5×10⁻⁸ M or less, about 1×10⁻⁸ M or less, about 5×10⁻⁹ M or less, or about 1×10⁻⁹ M or less.

Binding of an extracellular antigen-binding domain (for example, an scFv or an analog thereof) of an antigen-targeted CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a gamma counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet). In certain embodiments, the extracellular antigen-binding domain, the spacer region, or the intracellular signaling domain of the CAR is labeled with an epitope tag. Non-limiting examples of epitope tags include hemagglutinin (HA), c-Myc, FLAG, 6-His, and V5.

In certain embodiments, the extracellular antigen-binding domain specifically binds to an antigen. In certain embodiments, the extracellular antigen-binding domain is an scFv. In certain embodiments, the scFv is a human scFv. In certain embodiments, the scFv is a humanized scFv. In certain embodiments, the extracellular antigen-binding domain is a Fab, which is optionally crosslinked. In certain embodiments, the extracellular binding domain is a F(ab′)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. In certain embodiments, the antigen is a tumor antigen. In certain embodiments, the antigen is a pathogen antigen, including for example, a viral or a bacterial antigen.

In certain embodiments, the extracellular binding domain is an scFv that specifically binds to a tumor-specific, tumor-associated, or stroma antigen. The tumor-specific, tumor-associated, or stroma antigen can be for example, AFP, ALPP, ALPP2, ANTXR1, alpha-V beta-3 integrin, alpha-V beta-6 integrin, AXL, BCMA, B7-H3 (CD276), B7-H4 (VTCN1), carbonic anhydrase IX (CA1X), carcinoembryonic antigen (CEA), CD5, CD8, CD7, CD10, CD19, CD20, CD22, CD30, CD33, CD34, CD38, CD41, CD44, CD44V6, CD47, CD49c, CD49e, CD49f, CD56, CD61, CD66c, CD70, CD72, CD73, CD74, CD80, CD86, CD104, CD123, CD126, CD133, CD138, CD142, CD147, CD318, an antigen of a cytomegalovirus (CMV) infected cell (e.g., a cell surface antigen), CEA, cutaneous lymphocyte-associated antigen (CLA; a specialized glycoform of P-selectin glycoprotein ligand-1 (PSGL-1)), chorotoxin ligands, Claudin 6 (CLDN6), claudin 18.2 (CLDN18.2), CLL1, CRLF2, DLL-3, DR4, DR5, EGF1R, epidermal growth factor receptor (EGFR), EGFR806, EGFRvIII, epithelial glycoprotein-2 (EGP-2), epithelial glycoprotein-40 (EGP-40), epithelial cell adhesion molecule (EpCAM), EpHA2, receptor tyrosine-protein kinases erb-B2,3,4 (erb-B2,3,4), ERBB, ERBB2, folate-binding protein (FBP), fetal acetylcholine receptor (AChR), FAP, folate receptor-alpha (FOLR1), FZD1, FZD2, FZD3, FZD4, FZD5, FZD6, FZD7, FZD8, FZD9, FZD10, Ganglioside G2 (GD2), Ganglioside G3 (GD3), GFRA4, GP100, GPC2, GPC3, GSPG4, GUCY2C, human Epidermal Growth Factor Receptor 2 (HER2), human telomerase reverse transcriptase (hTERT), ICAM1 (CD54), Interleukin-13 receptor subunit alpha-2 (IL-13Ralpha2), kappa-light chain, kinase insert domain receptor (KDR), KLK2, Lewis Y (LeY), L1 cell adhesion molecule (L1CAM; (CD171), LMP1, LRRC15, melanoma antigen family A, 1 (MAGE-A1), MAGEA3, MAGEA4, MART1, mesothelin (MSLN), MET (c-Met; HGFR), MG7, mucin 1 (MUC1), TnMUC1, MUC3A, mucin 16 (MUC16), NECTIN4, NKGD2,an NKG2D ligand (for example, MIC-1, MIC-B, ULBP1, ULBP2, ULBP3, ULBP4, ULBP5, ULBP6), cancer-testis antigen NY-ESO-1, oncofetal antigen (h5T4), p53, PD-1, PD-L1, PD-L2, Proteinase3 (PR1), PRAME, prostate stem cell antigen (PSCA), prostate-specific membrane antigen (PSMA), REG3A (PAP), ROR1, ROR2, Survivin, Tyrosinase, tumor associated glycoprotein 72 (TAG-72), TROP2, tetraspanin 8 (TSPAN8), vascular endothelial growth factor R2 (VEGF-R2), or Wilms tumor protein (WT-1).

In a certain embodiments, the tumor specific target antigen is CD19, such as an scFv derived from the FMC63 antibody or 4G7 antibody. In some embodiments, the scFv comprises the CDRs of the FMC63 antibody: a CDRL1 sequence of RASQDISKYLN (SEQ ID NO:1), a CDRL2 sequence of SRLHSGV (SEQ ID NO:2), a CDRL3 sequence of GNTLPYTFG (SEQ ID NO:3), a CDRH1 sequence of DYGVS (SEQ ID NO:4), a CDRH2 sequence of VTWGSETTYYNSALKS (SEQ ID NO:5), and a CDRH3 sequence of YAMDYWG (SEQ ID NO:6); or a CDRL1 sequence of RASQDISKYLN (SEQ ID NO:1), a CDRL2 sequence of SRLHSGV (SEQ ID NO:2), a CDRL3 sequence of GNTLPYTFG (SEQ ID NO:3), a CDRH1 sequence of DYGVS (SEQ ID NO:4), a CDRH2 sequence of DNSKSQ (SEQ ID NO:63), and a CDRH3 sequence of YAMDYWG (SEQ ID NO:6). In some embodiments, an extracellular binding domain is an scFv derived from or comprising the heavy and light chain variable regions of antibody FMC63. The heavy and light chain variable regions of antibody FMC63 are shown in SEQ ID NO:64 and SEQ ID NO:65, respectively.

SEQ ID NO:64
FMC63 heavy chain variable region
EVKLQESGPGLVAPSQSLSVTCTVSGVSLPDYGVSW
IRQPPRKGLEWLGVIWGSETTYYNSALKSRLTIIKD
NSKSQVFLKMNSLQTDDTAIYYCAKHYYYGGSYAMD
YWGQGTSVTVSS
SEQ ID NO:65
FMC63 light chain variable region
DIQMTQTTSSLSASLGDRVTISCRASQDISKYLNWY
QQKPDGTVKLLIYHTSRLHSGVPSRFSGSGSGTDYS
LTISNLEQEDIATYFCQQGNTLPYTFGGGTKLEIT

In some embodiments, an scFv comprises the heavy and light chain CDRs of the CD19 monoclonal antibody 4G7, as set forth in SEQ ID NO:25 and SEQ ID NO:26 or SEQ ID NO: 27. In some embodiments, the extracellular antigen binding comprises the heavy and light chain variable region CDRs set forth in SEQ ID NO:25 and 26, or in SEQ NO: 25 and 27. In some embodiments, an scFv is derived from the CD19 monoclonal antibody 4G7, preferably comprises a part of the binding domains of CD19 monoclonal antibody 4G7, portions of variable region of the CD19 monoclonal antibody 4G7 immunoglobulin gamma 1 heavy chain (SEQ ID NO:25) and the variable fragments of the CD19 monoclonal antibody 4G7 immunoglobulin kappa light chain (SEQ ID NO:26 or SEQ ID NO:27) linked together by a flexible linker. In a particular embodiment, the flexible linker has the amino acid sequence set forth in SEQ ID NO:28. In some embodiments, the extracellular antigen binding comprises the heavy and light chain variable region CDRs set forth in SEQ ID NO: 25 and SEQ ID NO:26, or in SEQ NO: 25 and SEQ ID NO:27.

SEQ ID NO: 25 anti-human CD19 monoclonal antibody
4G7 heavy chain variable region:
EVQLQQSGPELIKPGASVKMSCKASGYTFTSYVMHWVKQKPGQGLEWIGY
INPYNDGTKYNEKFKGKATLTSDKSSSTAYMELSSLTSEDSAVYYCARGT
YYYGSRVFDYWGQGTTLTVSS
SEQ ID NO: 26 anti-human CD19 monoclonal antibody
4G7 immunoglobulin kappa light chain variable
region:
DIVMTQAAPSIPVTPGESVSISCRSSKSLLNSNGNTYLYWFLQRPGQSPQ
LLIYRMSNLASGVPDRFSGSGSGTAFTLRISRVEAEDVGVYYCMQHLEYP
FTFGAGTKLELKRAD
SEQ ID NO: 27 anti-human CD19 monoclonal antibody
4G7 immunoglobulin kappa light chain variable
region:
DIVMTQAAPSIPVTPGESVSISCRSSKSLLNSNGNTYLYWFLQRPGQSPQ
LLIYRMSNLASGVPDRFSGSGSGTAFTLRISRVEAEDVGVYYCMQHLEYP
FTFGAGTKLELKRSDP
flexible linker
SEQ ID NO: 28
GGGGSGGGGSGGGGS

In some embodiments, an scFv is derived from a CLA (cutaneous lymphocyte antigen) monoclonal antibody and comprises the heavy and light chain variable region of the CLA monoclonal antibody set forth in SEQ ID NO:29 and SEQ ID NO:30, respectively (see US Patent Application Publication No. 2019/0209611; incorporated herein by reference). In some embodiments, the extracellular antigen binding domain comprises the heavy and light chain variable region CDRs set forth in SEQ ID NO:29 and SEQ ID NO:30.

CLA VH (heavy chain variable region)
SEQ ID NO: 29
EVQLVESGGGLVQPGNSLKLSCSASGFTFSSYGMHWIRQAPGEGLDWVAY
ISSSSGTVYADAVKARFTISRDNAKNTLYLQLNSLKSEDTAIYYCARAQN
WDLFDYWGQGVMVTVSS
CLA VL (light chain variable region)
SEQ ID NO: 30
QIMLTQQAESLWISPGERVSITCRASQSLLYTDGKHYLSWYQQKPGQTTK
ALIYHASVRTDGVPTRFIGSGSGTEFTLSIEHVQPEDFAIYYCLQTLKSP
FTFGSGTKLEIK

In some embodiments, an scFv is derived from a CD142 monoclonal antibody and comprises the heavy and light chain variable region of the CD142 monoclonal antibody set forth in SEQ ID NOs: 31 and 32, respectively (see US Patent Application Publication No. 2019/0209611, incorporated herein by reference). In some embodiments, the extracellular antigen binding domain comprises the heavy and light chain variable region CDRs set forth in SEQ ID NO:31 and SEQ ID NO:32.

CD142 VH (heavy chain variable region)
SEQ ID NO: 31
QVQLKQSGPGLVQPSQSLSITCTVSGFSLSNYGVHWVRQSPGKGLEWLGV
IWSGGSTDYNVAFISRLIITKDNSKSQVFLKMNSLQADDTAIYFCARTTG
SVFNAMDHWGQGTSVTVSS
CD142 VL (light chain variable region)
SEQ ID NO: 32
QIVLTQSPALMSASPGEKVTMTCSASSSVTYMYWYQQKPRSSPKPWIYLT
SNLASGVPARFSGSGSGTSYSLTISSVEAEDAATYYCQQWSSNPLTFGAG
TKLELK

In some embodiments, an scFv is derived from a CD73 monoclonal antibody and comprises the heavy and light chain variable region of the CD73 monoclonal antibody set forth in SEQ ID NOs: 33 and 34, respectively (see US Patent Application Publication No. 2019/0209611, incorporated herein by reference). In some embodiments, the extracellular antigen binding domain comprises the heavy and light chain variable region CDRs set forth in SEQ ID NO:33 and SEQ ID NO:34.

CD73 VH (heavy chain variable region)
SEQ ID NO: 33
EVQLQQSGAELVKPGASVKLSCTASGFNIKDTYIHWVKQRPEQGLEWIGR
IDPATGNTEYDPKFQGKATITADTSSNTAYLHLSSLTSEDTAVYYCARGY
YGSSYPPWFAYWGQGTLVTVSA
CD73 VL (light chain variable region)
SEQ ID NO: 34
DIVMTQSHKFMSTSVGDRVSITCKASQDVGSAVAWYQQKPGQSPKLLIYW
ASTRHTGVPDRFTGSGSGTDFTLTISNVQSEDLADYFCQQYSSYPLTFGA
GTKLELK

In some embodiments, an scFv is derived from a CD49c monoclonal antibody and comprises the heavy and light chain variable region of the CD49c monoclonal antibody set forth in SEQ ID NOs: 35 and 36, respectively (see US Patent Application Publication No. 2019/0209611; incorporated herein by reference). In some embodiments, the extracellular antigen binding domain comprises the heavy and light chain variable region CDRs set forth in SEQ ID NO:35 and SEQ ID NO:36.

CD49c VH (heavy chain variable region)
SEQ ID NO: 35
EVQLQQSGAELVKPGASVKLSCTASGFNIKDTYMHWVKQRPEQGLEWIGR
IDPANGHTKYDPKFQGKATITADTSSNAAYLQLNSLTSEDTAVYYCARRV
AYAMDYWGQGTSVTVSS
CD49c VL (light chain variable region)
SEQ ID NO: 36
ENVLTQSPAIMSASPGEKVTMTCSASSSVTYMHWYQQKSSTSPKLWIYDT
SKLASGVPGRFSGSGSGNSYSLTISSMEAEDVATYCCFQGSGYPLTFGGG
TKLEIK

In some embodiments, an scFv is derived from a CD66c monoclonal antibody and comprises the heavy and light chain variable region of the CD66c monoclonal antibody set forth in SEQ ID NOs: 37 and 38, respectively (see US Patent Application Publication No. 2019/0209611, incorporated herein by reference). In some embodiments, the extracellular antigen binding domain comprises the heavy and light chain variable region CDRs set forth in SEQ ID NO:37 and SEQ ID NO:38.

CD66c VH (heavy chain variable region)
SEQ ID NO: 37
QVTLKESGPGILKPSQTLSLTCSFSGFSLSTSGMGVGWIRQPSGKSLEWL
AHIWWNDERYYNPSLKNQLTISKDTSRNQVFLKITSVDTADTATYYCARS
PRGYFDYWGHGTTLTVSS
CD66c VL (light chain variable region)
SEQ ID NO: 38
DIVMTQSQKFMSTSVGDRVSVTCKASQNVVTNVAWYQQTPGQSPKALIYS
ASYRYSGVPDRFSGSGSGTDFTLTISNVQSGDLAEYFCQQYNSYPLTFGA
GTKLELK

In some embodiments, an scFv is derived from a CD104 monoclonal antibody and comprises the heavy and light chain variable region of the CD104 monoclonal antibody set forth in SEQ ID NOs: 39 and 40, respectively (see US Patent Application Publication No. 2019/0209611; incorporated herein by reference). In some embodiments, the extracellular antigen binding domain comprises the heavy and light chain variable region CDRs set forth in SEQ ID NO:39 and SEQ ID NO:40.

CD104 VH (heavy chain variable region)
SEQ ID NO: 39
QVNLLQSGAALVKPGASVKLSCKASGYTFTDYYIFWVKQSHGKSLEWIGY
INPNSGSTNYNEKFKRKATLSVDKSTNTAYMELSRLTSEDSATYYCTRRA
YYGYNPFDYWGQGVMVTVSS
CD104 VL (light chain variable region)
SEQ ID NO: 40
DIQMTQTPSSMPASLGERVTISCRASRGINNYLSWYQQNLDGTIKPLIYY
TSNLQSGVPSRFSGSGSGTDYSLTISSLEPEDFAMYYCQQYDSSPWTFGG
GTKLELK

In some embodiments, an scFv is derived from a CD318 monoclonal antibody and comprises the heavy and light chain variable region of the CD318 monoclonal antibody set forth in SEQ ID NO:41 and SEQ ID NO:42, respectively (see US Patent Application Publication No. 2019/0209611; incorporated herein by reference). In some embodiments, the extracellular antigen binding domain comprises the heavy and light chain variable region CDRs set forth in SEQ ID NO:41 and SEQ ID NO:42.

CD318 VH (heavy chain variable region)
SEQ ID NO: 41
EVQLQQSGAELVRPGALVKLSCKASGFNIKDYYIHWVKQRPEQGLEWIGW
IDPENGHTIYDPKFQGKASITADTSSNTAYLQLSSLTSEDTAVYYCARLT
GTTYAMDYWGQGTSVTVSS
CD318 VL (light chain variable region)
SEQ ID NO: 42
DIVMTQSHKFMSTSVGDRVSITCKASQDVSTAVAWYQQKSGQSPKLLIYW
ASTRHTGVPDRFTGSGSGTDYTLTISSVQAEDLALYYCQQHYSTPYTFGG
GTKLEIK

In some embodiments, an scFv is derived from a TSPAN8 monoclonal antibody and comprises the heavy and light chain variable region of the TSPAN8 monoclonal antibody set forth in SEQ ID NOs: 43 and 44, respectively (see US Patent Application Publication No. 2019/0209611; incorporated herein by reference). In some embodiments, the extracellular antigen binding domain comprises the heavy and light chain variable region CDRs set forth in SEQ ID NO:43 and SEQ ID NO:44.

TSPAN8 VH (heavy chain variable region)
SEQ ID NO: 43
EVKLLESGGGLVQPGGSMRLSCAASGFTFTDFYMNWIRQPAGKAPEWLGF
IRNKASGYTTEYNPSVKGRFTISRDNTQNMLYLQMNTLRAEDTATYYCAR
AHSYYGYDYFDYWGQGVMVTVSS
TSPAN8 VL (light chain variable region)
SEQ ID NO: 44
DIQMTQSPASLSASLEEIVTITCQASQDIGNWLSWYQQKPGKSPQLLIYG
ATSLADGVPSRFSGSRSGTQYSLKISRLQVEDIRIYYCLQAYSAPWTFGG
GTKLELK

In some embodiments, an scFv to CLA has the amino acid sequence set forth in SEQ ID NO:45 or SEQ ID NO:46 (see US Patent Application Publication No. 2019/0209611; incorporated herein by reference).

CLA specific scFv VH-linker-VL
SEQ ID NO: 45
EVQLVESGGGLVQPGNSLKLSCSASGFTFSSYGMHWIRQAPGEGLDWVAY
ISSSSGTVYADAVKARFTISRDNAKNTLYLQLNSLKSEDTAIYYCARAQN
WDLFDYWGQGVMVTVSSGGGGSGGGGSGGGGSQIMLTQQAESLWISPGER
VSITCRASQSLLYTDGKHYLSWYQQKPGQTTKALIYHASVRTDGVPTRFI
GSGSGTEFTLSIEHVQPEDFAIYYCLQTLKSPFTFGSGTKLEIK
CLA specific scFv VL-linker-VH
SEQ ID NO: 46
QIMLTQQAESLWISPGERVSITCRASQSLLYTDGKHYLSWYQQKPGQTTK
ASVRTDGVPTRFIGSGSGTEFTLSIEHVQPEDFAIYYCLQTLKSPFTFGS
ALIYHGTKLEIKGGGGSGGGGSGGGGSEVQLVESGGGLVQPGNSLKLSCS
ASGFTFSSYGMHWIRQAPGEGLDWVAYISSSSGTVYADAVKARFTISRDN
AKNTLYLQLNSLKSEDTAIYYCARAQNWDLFDYWGQGVMVTVSS

In some embodiments, an scFv to CD142 has the amino acid sequence set forth in SEQ ID NO:47 or SEQ ID NO:48 (see US Patent Application Publication No. 2019/0209611; incorporated herein by reference).

CD142 specific CAR sequence VH-linker-VL
SEQ ID NO: 47
QVQLKQSGPGLVQPSQSLSITCTVSGFSLSNYGVHWVRQSPGKGLEWLGV
IWSGGSTDYNVAFISRLIITKDNSKSQVFLKMNSLQADDTAIYFCARTTG
SVFNAMDHWGQGTSVTVSSGGGGSGGGGSGGGGSQIVLTQSPALMSASPG
EKVTMTCSASSSVTYMYWYQQKPRSSPKPWIYLTSNLASGVPARFSGSGS
GTSYSLTISSVEAEDAATYYCQQWSSNPLTFGAGTKLELK
CD142 specific CAR sequence VL-linker-VH
SEQ ID NO: 48
QIVLTQSPALMSASPGEKVTMTCSASSSVTYMYWYQQKPRSSPKPWIYLT
SNLASGVPARFSGSGSGTSYSLTISSVEAEDAATYYCQQWSSNPLTFGAG
TKLELKGGGGSGGGGSGGGGSQVQLKQSGPGLVQPSQSLSITCTVSGFSL
SNYGVHWVRQSPGKGLEWLGVIWSGGSTDYNVAFISRLIITKDNSKSQVF
LKMNSLQADDTAIYFCARTTGSVFNAMDHWGQGTSVTVSS

In some embodiments, an scFv to CD73 has the amino acid sequence set forth in SEQ ID NO:49 or SEQ ID NO:50 (see US Patent Application Publication No. 2019/0209611; incorporated herein by reference).

CD73 specific CAR sequence VH-linker-VL
SEQ ID NO: 49
EVQLQQSGAELVKPGASVKLSCTASGFNIKDTYIHWVKQRPEQGLEWIGR
IDPATGNTEYDPKFQGKATITADTSSNTAYLHLSSLTSEDTAVYYCARGY
YGSSYPPWFAYWGQGTLVTVSAGGGGSGGGGSGGGGSDIVMTQSHKFMST
SVGDRVSITCKASQDVGSAVAWYQQKPGQSPKLLIYWASTRHTGVPDRFT
GSGSGTDFTLTISNVQSEDLADYFCQQYSSYPLTFGAGTKLELK
CD73 specific CAR sequence VL-linker-VH
SEQ ID NO: 50
DIVMTQSHKFMSTSVGDRVSITCKASQDVGSAVAWYQQKPGQSPKLLIYW
ASTRHTGVPDRFTGSGSGTDFTLTISNVQSEDLADYFCQQYSSYPLTFGA
GTKLELKGGGGSGGGGSGGGGSEVQLQQSGAELVKPGASVKLSCTASGFN
IKDTYIHWVKQRPEQGLEWIGRIDPATGNTEYDPKFQGKATITADTSSNT
AYLHLSSLTSEDTAVYYCARGYYGSSYPPWFAYWGQGTLVTVSA

In some embodiments, an scFv to CD49c has the amino acid sequence set forth in SEQ ID NO:51 or SEQ ID NO:52 (see US Patent Application Publication No. 2019/0209611; incorporated herein by reference).

CD49c specific CAR sequence VH-linker-VL
SEQ ID NO: 51
EVQLQQSGAELVKPGASVKLSCTASGFNIKDTYMHWVKQRPEQGLEWIGR
IDPANGHTKYDPKFQGKATITADTSSNAAYLQLNSLTSEDTAVYYCARRV
AYAMDYWGQGTSVTVSSGGGGSGGGGSGGGGSENVLTQSPAIMSASPGEK
VTMTCSASSSVTYMHWYQQKSSTSPKLWIYDTSKLASGVPGRFSGSGSGN
SYSLTISSMEAEDVATYCCFQGSGYPLTFGGGTKLEIK
CD49c specific CAR sequence VL-linker-VH
SEQ ID NO: 52
ENVLTQSPAIMSASPGEKVTMTCSASSSVTYMHWYQQKSSTSPKLWIYDT
SKLASGVPGRFSGSGSGNSYSLTISSMEAEDVATYCCFQGSGYPLTFGGG
TKLEIKGGGGSGGGGSGGGGSEVQLQQSGAELVKPGASVKLSCTASGFNI
KDTYMHWVKQRPEQGLEWIGRIDPANGHTKYDPKFQGKATITADTSSNAA
YLQLNSLTSEDTAVYYCARRVAYAMDYWGQGTSVTVSS

In some embodiments, an scFv to CD66c has the amino acid sequence set forth in SEQ ID NO:53 or SEQ ID NO:54 (see US Patent Application Publication No. 2019/0209611; incorporated herein by reference).

CD66c specific CAR sequence VH-linker-VL
SEQ ID NO: 53
QVTLKESGPGILKPSQTLSLTCSFSGFSLSTSGMGVGWIRQPSGKSLEWL
AHIWWNDERYYNPSLKNQLTISKDTSRNQVFLKITSVDTADTATYYCARS
PRGYFDYWGHGTTLTVSSGGGGSGGGGSGGGGSDIVMTQSQKFMSTSVGD
RVSVTCKASQNVVTNVAWYQQTPGQSPKALIYSASYRYSGVPDRFSGSGS
GTDFTLTISNVQSGDLAEYFCQQYNSYPLTFGAGTKLELK
CD66c specific CAR sequence VL-linker-VH
SEQ ID NO: 54
DIVMTQSQKFMSTSVGDRVSVTCKASQNVVTNVAWYQQTPGQSPKALIYS
ASYRYSGVPDRFSGSGSGTDFTLTISNVQSGDLAEYFCQQYNSYPLTFGA
GTKLELKGGGGSGGGGSGGGGSQVTLKESGPGILKPSQTLSLTCSFSGFS
LSTSGMGVGWIRQPSGKSLEWLAHIWWNDERYYNPSLKNQLTISKDTSRN
QVFLKITSVDTADTATYYCARSPRGYFDYWGHGTTLTVSS

In some embodiments, an scFv to CD104 has the amino acid sequence set forth in SEQ ID NO:55 or SEQ ID NO:56 (see US Patent Application Publication No. 2019/0209611; incorporated herein by reference).

CD104 specific CAR sequence VH-linker-VL
SEQ ID NO: 55
QVNLLQSGAALVKPGASVKLSCKASGYTFTDYYIFWVKQSHGKSLEWIGY
INPNSGSTNYNEKFKRKATLSVDKSTNTAYMELSRLTSEDSATYYCTRRA
YYGYNPFDYWGQGVMVTVSSGGGGSGGGGSGGGGSDIQMTQTPSSMPASL
GERVTISCRASRGINNYLSWYQQNLDGTIKPLIYYTSNLQSGVPSRFSGS
GSGTDYSLTISSLEPEDFAMYYCQQYDSSPWTFGGGTKLELK
CD104 specific CAR sequence VL-linker-VH
SEQ ID NO: 56
DIQMTQTPSSMPASLGERVTISCRASRGINNYLSWYQQNLDGTIKPLIYY
TSNLQSGVPSRFSGSGSGTDYSLTISSLEPEDFAMYYCQQYDSSPWTFGG
GTKLELKGGGGSGGGGSGGGGSQVNLLQSGAALVKPGASVKLSCKASGYT
FTDYYIFWVKQSHGKSLEWIGYINPNSGSTNYNEKFKRKATLSVDKSTNT
AYMELSRLTSEDSATYYCTRRAYYGYNPFDYWGQGVMVTVSS

In some embodiments, an scFv to CD318 has the amino acid sequence set forth in SEQ ID NO:57 or SEQ ID NO:58 (see US Patent Application Publication No. 2019/0209611; incorporated herein by reference).

CD318 specific CAR sequence VH-linker-VL
SEQ ID NO: 57
EVQLQQSGAELVRPGALVKLSCKASGFNIKDYYIHWVKQRPEQGLEWIGW
IDPENGHTIYDPKFQGKASITADTSSNTAYLQLSSLTSEDTAVYYCARLT
GTTYAMDYWGQGTSVTVSSGGGGSGGGGSGGGGSDIVMTQSHKFMSTSVG
DRVSITCKASQDVSTAVAWYQQKSGQSPKLLIYWASTRHTGVPDRFTGSG
SGTDYTLTISSVQAEDLALYYCQQHYSTPYTFGGGTKLEIK
CD318 specific CAR sequence VL-linker-VH
SEQ ID NO: 58
DIVMTQSHKFMSTSVGDRVSITCKASQDVSTAVAWYQQKSGQSPKLLIYW
ASTRHTGVPDRFTGSGSGTDYTLTISSVQAEDLALYYCQQHYSTPYTFGG
GTKLEIKGGGGSGGGGSGGGGSEVQLQQSGAELVRPGALVKLSCKASGFN
IKDYYIHWVKQRPEQGLEWIGWIDPENGHTIYDPKFQGKASITADTSSNT
AYLQLSSLTSEDTAVYYCARLTGTTYAMDYWGQGTSVTVSS

In some embodiments, an scFv to TSPAN8 has the amino acid sequence set forth in SEQ ID NO:59 or SEQ ID NO:60 (see US Patent Application Publication No. 2019/0209611; incorporated herein by reference).

TSPAN8 specific CAR sequence VH-linker-VL
SEQ ID NO: 59
EVKLLESGGGLVQPGGSMRLSCAASGFTFTDFYMNWIRQPAGKAPEWLGF
IRNKASGYTTEYNPSVKGRFTISRDNTQNMLYLQMNTLRAEDTATYYCAR
AHSYYGYDYFDYWGQGVMVTVSSGGGGSGGGGSGGGGSDIQMTQSPASLS
ASLEEIVTITCQASQDIGNWLSWYQQKPGKSPQLLIYGATSLADGVPSRF
SGSRSGTQYSLKISRLQVEDIRIYYCLQAYSAPWTFGGGTKLELK
TSPAN8 specific CAR sequence VL-linker-VH
SEQ ID NO: 60
DIQMTQSPASLSASLEEIVTITCQASQDIGNWLSWYQQKPGKSPQLLIYG
ATSLADGVPSRFSGSRSGTQYSLKISRLQVEDIRIYYCLQAYSAPWTFGG
GTKLELKGGGGSGGGGSGGGGSEVKLLESGGGLVQPGGSMRLSCAASGFT
FTDFYMNWIRQPAGKAPEWLGFIRNKASGYTTEYNPSVKGRFTISRDNTQ
NMLYLQMNTLRAEDTATYYCARAHSYYGYDYFDYWGQGVMVTVSS

### Transmembrane Domain of a CAR

In some embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. The transmembrane domain of the CAR can comprise, for example, a CD8 polypeptide, a CD28 polypeptide, a NKG2D polypeptide, a CD3zeta polypeptide, a CD4 polypeptide, a 4-1BB polypeptide, an OX40 polypeptide, an ICOS polypeptide, a synthetic peptide (not based on a protein associated with the immune response), or a combination thereof. In some embodiments, the transmembrane domain of a CAR comprises the transmembrane domain of the molecule, or portion of the molecule, used in the membrane proximal region of the intracellular signaling domain of the CAR.

In certain embodiments, the transmembrane domain comprises a CD8 polypeptide. In certain embodiments, the CD8 polypeptide has an amino acid sequence that is at least about 85%, about 90%, about 95%, about 96%, about 97%, about 98%, about 99% or about 100% identical to the sequence having a NCBI Reference No: NP_001139345.1, SEQ ID NO:7, (sequence identity herein may be determined using standard software such as BLAST or FASTA), or fragments thereof, and/or may optionally comprise up to one or up to two or up to three conservative amino acid substitutions. As used herein, a “conservative amino acid substitution” means an amino acid substitution wherein an amino acid is substituted for another electronically similar amino acid. For example, an amino acid with a hydrophobic side chain can be substituted for a different amino acid also having a hydrophobic side chain (e.g., leucine substituted for isoleucine, alanine substituted for valine, and the like); an amino acid with an acidic chain can be substituted for a different amino acid also having an acidic side chain (e.g., aspartic acid substituted for glutamic acid, and the like); an amino acid with a basic chain can be substituted for a different amino acid also having a basic side chain (e.g., lysine substituted for arginine, and the like); and an amino acid with a polar side chain can be substituted for a different amino acid also having a polar side chain (e.g., serine substituted for threonine, and the like). In certain embodiments, the CD8 polypeptide can have an amino acid sequence that is a consecutive portion of SEQ ID NO:7 which is at least 20, or at least 30, or at least 40, or at least 50, and up to 235 amino acids in length. Alternatively, or additionally, in non-limiting various embodiments, the CD8 polypeptide comprises or has an amino acid sequence of amino acids 1 to 235, 1 to 50, 50 to 100, 100 to 150, 150 to 200, or 200 to 235 of SEQ ID NO:7. In certain embodiments, a CAR comprises a transmembrane domain comprising a human CD8 polypeptide that comprises an amino acid sequence of amino acids 137 to 209 of SEQ ID NO: 7.

SEQ ID NO: 7
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

In certain embodiments, the CD8 polypeptide has an amino acid sequence that is at least about 85%, about 90%, about 95%, about 96%, about 97%, about 98%, about 99% or about 100% identical to the sequence having a NCBI Reference No: AAA92533.1, SEQ ID NO:8, or fragments thereof, and/or may optionally comprise up to one or up to two or up to three conservative amino acid substitutions. In certain embodiments, the CD8 polypeptide can have an amino acid sequence that is a consecutive portion of SEQ ID NO:8 which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, 151 to 219, or 200 to 247 of SEQ ID NO: 8. In certain embodiments, the CAR comprises a transmembrane domain comprising a murine CD8 polypeptide that comprises an amino acid sequence of amino acids 151 to 219 of SEQ ID NO:8.

SEQ ID NO: 8
MASPLTRELSLNLLLMGESIILGSGEAKPQAPELRIFPKKMDAELGQKVD
LVCEVLGSVSQGCSWLFQNSSSKLPQPTFVVYMASSHNKITWDEKLNSSK
LFSAVRDTNNKYVLTLNKFSKENEGYYFCSVISNSVMYFSSVVPVLQKVN
STTTKPVLRTPSPVHPTGTSQPQRPEDCRPRGSVKGTGLDFACDIYIWAP
LAGICVAPLLSLIITLICYHRSRKRVCKCPRPLVRQEGKPRPSEKIV

In certain embodiments, the CD8 polypeptide comprises or has the amino acid sequence set forth in SEQ ID NO:9.

SEQ ID NO: 9
STTTKPVLRTPSPVHPTGTSQPQRPEDCRPRGSVKGTGLDFACDIYIWAP
LAGICVALLLSLIITLICY

In certain embodiments, the transmembrane domain of a CAR comprises a CD28 polypeptide. The CD28 polypeptide can have an amino acid sequence that is at least about 85%, about 90%, about 95%, about 96%, about 97%, about 98%, about 99% or 100% identical to the sequence having a NCBI Reference No: P10747 or NP_006130 (SEQ ID No: 10), or fragments thereof, and/or may optionally comprise up to one or up to two or up to three conservative amino acid substitutions. In non-limiting certain embodiments, the CD28 polypeptide can have an amino acid sequence that is a consecutive portion of SEQ ID NO: 10 which is at least 20, or at least 30, or at least 40, or at least 50, and up to 220 amino acids in length. Alternatively, or additionally, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO:10. In certain embodiments, the CD28 polypeptide comprised in the transmembrane domain of a presently disclosed CAR has an amino acid sequence of amino acids 153 to 179 of SEQ ID NO: 10.

SEQ ID NO: 10
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

In certain embodiments, the transmembrane domain of a CAR comprises a NKG2D polypeptide. The NKG2D polypeptide can have an amino acid sequence that is at least about 85%, about 90%, about 95%, about 96%, about 97%, about 98%, about 99% or 100% identical to the sequence having a NCBI Reference No: NP_031386.2 (SEQ ID NO: 66), or fragments thereof, and/or may optionally comprise up to one or up to two or up to three conservative amino acid substitutions. In non-limiting certain embodiments, the NKG2D polypeptide can have an amino acid sequence that is a consecutive portion of SEQ ID NO: 66 which is at least 20, or at least 30, or at least 40, or at least 50, and up to 216 amino acids in length. In certain embodiments, the NKG2D polypeptide comprises the transmembrane domain of SEQ ID NO: 66 (e.g., amino acids 52-72).

SEQ ID NO: 66
MGWIRGRRSRHSWEMSEFHNYNLDLKKSDFSTRWQKQRCPVVKSKCRENA
SPFFFCCFIAVAMGIRFIIMVTIWSAVFLNSLFNQEVQIPLTESYCGPCP
KNWICYKNNCYQFFDESKNWYESQASCMSQNASLLKVYSKEDQDLLKLVK
SYHWMGLVHIPTNGSWQWEDGSILSPNLLTIIEMQKGDCALYASSFKGYI
ENCSTPNTYICMQRTV

In some embodiments, the transmembrane domain of a CAR comprises a CD3zeta (CD3ζ) polypeptide. The CD3zeta polypeptide can have an amino acid sequence that is at least about 85%, about 90%, about 95%, about 96%, about 97%, about 98%, about 99% or 100% identical to the sequence of the transmembrane domain of CD3zeta set forth in NCBI Reference No: NP_932170 (SEQ ID NO:11), SEQ ID NO: 12, or SEQ ID NO:13, and/or may optionally comprise up to one or up to two or up to three conservative amino acid substitutions.

In some embodiments, the transmembrane domain of a CAR comprises a CD4 polypeptide. The CD4 polypeptide can have an amino acid sequence that is at least about 85%, about 90%, about 95%, about 96%, about 97%, about 98%, about 99% or 100% identical to the sequence of the transmembrane domain of CD4 set forth in NCBI Reference No: NP_000607.1, NP_001181943.1 or NP_001181946.1, (each incorporated herein by reference) and/or may optionally comprise up to one or up to two or up to three conservative amino acid substitutions.

In some embodiments, the transmembrane domain of a CAR comprises a 4-1BB polypeptide. The 4-1BB polypeptide can have an amino acid sequence that is at least about 85%, about 90%, about 95%, about 96%, about 97%, about 98%, about 99% or 100% identical to the sequence of the transmembrane domain of 4-1BB set forth in NCBI Reference No: P41273 (incorporated herein by reference) or NP_001552.2 (SEQ ID NO: 18), and/or may optionally comprise up to one or up to two or up to three conservative amino acid substitutions.

In some embodiments, the transmembrane domain of a CAR comprises an OX40 polypeptide. The OX40 polypeptide can have an amino acid sequence that is at least about 85%, about 90%, about 95%, about 96%, about 97%, about 98%, about 99% or 100% identical to the sequence of the transmembrane domain of OX40 set forth in NCBI Reference No: NP_003318.1 (incorporated herein by reference), and/or may optionally comprise up to one or up to two or up to three conservative amino acid substitutions.

In some embodiments, the transmembrane domain of a CAR comprises an ICOS polypeptide. The ICOS polypeptide can have an amino acid sequence that is at least about 85%, about 90%, about 95%, about 96%, about 97%, about 98%, about 99% or 100% identical to the sequence of the transmembrane domain of ICOS set forth in NCBI Reference No: NP_036224.1 (incorporated herein by reference), and/or may optionally comprise up to one or up to two or up to three conservative amino acid substitutions.

### Spacer Region of a CAR

In certain non-limiting embodiments, a CAR can also comprise a spacer region that links the extracellular antigen-binding domain to the transmembrane domain. The spacer region can be flexible enough to allow the antigen binding domain to orient in different directions to facilitate antigen recognition. The spacer region can be the hinge region from IgG1 (GenPept Ref No.: P01857.1, incorporated herein by reference), or the CH2CH3 region of an immunoglobulin (e.g., IgG4 (GenPept Ref No.: P01861.1, incorporated herein by reference), a portion of a CD3 polypeptide, a portion of a CD28 polypeptide (e.g., a portion of SEQ ID NO: 10), a portion of a CD8 polypeptide (e.g., a portion of SEQ ID NO: 7, or a portion of SEQ ID NO:8), a variation of any of the foregoing which is at least about 80%, at least about 85%, at least about 90%, or at least about 95% identical thereto, or a synthetic spacer sequence.

### Intracellular Signaling Domain or Domains of a CAR

As used herein, the term “intracellular signaling domain” refers to the intracellular domain of a CAR which comprises one or more cytoplasmic or intracellular signaling domains, or portions of cytoplasmic of intracellular signaling domains, of cell surface or intracellular molecules such as activating or stimulatory molecules, co-stimulatory molecules, co-stimulatory ligands, and signal transduction molecules. As used herein, “activating or stimulatory molecules” refer to cell surface molecules such as antigen receptors or phagocytosis receptors or cytokine receptors or chemokine receptors, which can activate or stimulate a cell (e.g., a cell of the myeloid lineage, e.g., a monocyte and/or macrophage). As used herein, “co-stimulatory molecules” refer to cell surface molecules other than antigen receptors or their ligands that are required for an efficient response of immune cells to antigen, providing optimal cellular activation or suppression. As used herein, a “co-stimulatory ligand” refers to a protein expressed on the cell surface that upon binding to its co-stimulatory receptor produces a co-stimulatory response, i.e., an intracellular response that effects the stimulation provided when an antigen binds to its receptor. As used herein, “signal transduction molecules” refer to intracellular proteins, such as signaling adaptors, that participate in the propagation of a signaling cascade in response to binding of extracellular or intracellular receptors to their cognate ligands.

In certain non-limiting embodiments, an intracellular signaling domain of the CAR can comprise one or more intracellular signaling domains derived from stimulatory molecules, co-stimulatory molecules, co-stimulatory ligands or signal transduction molecules including but not limited to CD3zeta (CD3ζ), CD3delta, CD3epsilon, CD3gamma, CD4, CD8A, CD8B, CD2, CD7, LIGHT, CD27, CD28, 4-1BB (CD137), CD226 (DNAM1), B24 (CD244), ICOS (CD278), CTLA-4, GITR, OX40 (CD134), LAT, PD-1, TIM3, TIGIT, PD-L1, PD-L2, OX40L, 4-1BBL, ICOSLG, CD30L, CD30, CD36, CD68, CD40, CD70, CD80, CD83, CD86, CD163, CD204 (MSR1), CD206 (MRC1), CD209 (DC-SIGN), AGER (RAGE), CD276 (B7-H3), CD147, LILRB1, LILRB2, LILRB3, LILRB4, LILRB5, LILRA1, LILRA2, LILRA3, LILRA4, LILRA5, LILRA6, CLEC1A (CLEC1), CLEC1B (CLEC2), CLEC2A, CLEC2B, CLEC4D (DECTIN-3), CLEC4E (MINCLE), CLEC5A, CLEC6A (DECTIN-2), CLEC7A (DECTIN-1), CLEC8A (LOX-1)., CLEC9A (DNGR-1), CLEC10A (MGL), CLEC12A (MICL), SIGLEC1-11, SIGLEC14-16. AXL, MERTK, TYRO3, TREM2, CD11A (LFA-1; ITGAL), CD11B (ITGAM), CD11C (ITGAX), CSFIR (M-CSFR; CD115), GM-CSFR (CD116), CD14, CD16A (FcγRIIIa), CD32A (FcγRIIa), CD32B (FcγRIIb), CD32C (FcγRIIc), CD64 (FcγRI), FcRγ (FcεRIγ), MEGF10, CD79A, CD79B, CD19, TLR-1, TLR-2, TLR-3, TLR-4, TLR-5, TLR-6, TLR-7, TLR-8, TLR-9, MYD88, MAL, TRAM (TICAM2), TRIF (TICAM1), DAP-10, DAP-12, MAVS, STING, RIG-I, AIM2, NOD1-5, NLRP1-14, RIPK2, CASP1-10, CASP12-L, CASP12-S, CASP-14, cytokine receptors (including but not limited to interleukin (IL-) receptors, IFNGR, IFNGR1, IFNGR2, IFNAR, IFNAR1, IFNAR2, IFNLR1, CD116 (GM-CSFR), CSF2RA, CSF2RB, CSF1R (CD115; M-CSFR), IL10R, IL10RA, IL10RB, TGFBR, TGFBR1, TGFBR2. TNFRSF1A, TNFRSF1B), chemokine receptors (including but not limited to CCR1, CCR2, CCR3, CCR4, CCR5, CCR6, CCR7, CCR8, CCR9, CCR10, CCR11, CXCR1, CXCR2, CXCR3, CXCR3B, CXCR4, CXCR5, CXCR6, CXCR7, XCR1, or CX3CR1), or any combination thereof.

In certain non-limiting embodiments, an intracellular signaling domain of the CAR can comprise a CD3zeta (CD3) polypeptide. CD32 comprises 3 immunoreceptor tyrosine-based activation motifs (ITAMs) and transmits an activation signal to the cell (e.g., a cell of the myeloid lineage, e.g., a monocyte and/or macrophage) after antigen is bound. In certain embodiments, the CD3zeta polypeptide has an amino acid sequence that is at least about 85%, about 90%, about 95%, about 96%, about 97%, about 98%, about 99% or about 100% identical to the sequence having a NCBI Reference No: NP_932170 (SEQ ID NO: 11), or fragments thereof, and/or can optionally comprise up to one or up to two or up to three conservative amino acid substitutions. In certain non-limiting embodiments, the CD3 polypeptide comprises or has an amino acid sequence that is a consecutive portion of SEQ ID NO:11, which is at least 20, or at least 30, or at least 40, or at least 50, and up to 164 amino acids in length. Alternatively, or additionally, in non-limiting various embodiments, the CD3zeta polypeptide comprises or has an amino acid sequence of amino acids 1 to 164, 1 to 50, 50 to 100, 100 to 150, or 150 to 164 of SEQ ID NO:11. In certain embodiments, the CD3 polypeptide comprises or has an amino acid sequence of amino acids 52 to 164 of SEQ ID NO:11.

SEQ ID NO: 11
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

In certain embodiments, the CD3zeta polypeptide has an amino acid sequence that is at least about 85%, about 90%, about 95%, about 96%, about 97%, about 98%, about 99% or about 100% identical to the sequence having a NCBI Reference No: NP_001106864.2 (SEQ ID NO:12), or fragments thereof, and/or may optionally comprise up to one or up to two or up to three conservative amino acid substitutions. In certain non-limiting embodiments, the CD3zeta polypeptide can have an amino acid sequence that is a consecutive portion of SEQ ID NO:12, which is at least about 20, or at least about 30, or at least about 40, or at least about 50, or at least about 90, or at least about 100, and up to 188 amino acids in length. Alternatively, or additionally, in non-limiting various embodiments, the CD35 polypeptide comprises or has an amino acid sequence of amino acids 1 to 164, 1 to 50, 50 to 100, 52 to 142, 100 to 150, or 150 to 188 of SEQ ID NO: 12. In certain embodiments, the CD3zeta polypeptide comprises or has an amino acid sequence of amino acids 52 to 142 of SEQ ID NO:12.

SEQ ID NO: 12
MKWKVSVLACILHVRFPGAEAQSFGLLDPKLCYLLDGILFIYGVIITALY
LRAKFSRSAETAANLQDPNQLYNELNLGRREEYDVLEKKRARDPEMGGKQ
RRRNPQEGVYNALQKDKMAEAYSEIGTKGERRRGKGHDGLYQDSHFQAVQ
FGNRREREGSELTRTLGLRARPKACRHKKPLSLPAAVS

In certain embodiments, the CD3zeta polypeptide comprises or has the amino acid sequence set forth in SEQ ID NO:13.

SEQ ID NO: 13
RAKFSRSAETAANLQDPNQLYNELNLGRREEYDVLEKKRARDPEMGGKQQ
RRRNPQEGVYNALQKDKMAEAYSEIGTKGERRRGKGHDGLYQGLSTATKD
TYDALHMQTLAPR

In certain embodiments, the intracellular signaling domain of the CAR can comprise a CD28 polypeptide. In certain embodiments, the CD28 polypeptide can have an amino acid sequence that is at least about 85%, about 90%, about 95%, about 96%, about 97%, about 98%, about 99% or 100% identical to the sequence having a NCBI Reference No: P10747 (incorporated herein by reference) or NP_006130 (SEQ ID NO:10), or fragments thereof, and/or may optionally comprise up to one or up to two or up to three conservative amino acid substitutions. In non-limiting certain embodiments, the CD28 polypeptide has an amino acid sequence that is a consecutive portion of SEQ ID NO:10 which is at least 20, or at least 30, or at least 40, or at least 50, and up to 220 amino acids in length. Alternatively or additionally, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO:10. In certain embodiments, the intracellular signaling domain of the CAR comprises a signaling region that comprises a CD28 polypeptide having an amino acid sequence of amino acids 180 to 220 of SEQ ID NO: 10.

In certain embodiments, the CD28 polypeptide has an amino acid sequence that is at least about 85%, about 90%, about 95%, about 96%, about 97%, about 98%, about 99% or about 100% identical to the sequence having a NCBI Reference No: NP_031668.3 (SEQ ID NO:14), or fragments thereof, and/or may optionally comprise up to one or up to two or up to three conservative amino acid substitutions. In non-limiting certain embodiments, the CD28 polypeptide has an amino acid sequence that is a consecutive portion of SEQ ID NO:14 which is at least about 20, or at least about 30, or at least about 40, or at least about 50, and up to 218 amino acids in length. Alternatively or additionally, in non-limiting various embodiments, the CD28 polypeptide has an amino acid sequence of amino acids 1 to 218, 1 to 50, 50 to 100, 100 to 150, 114 to 220, 150 to 200, 178 to 218, or 200 to 220 of SEQ ID NO:14. In certain embodiments, the signaling region of a presently disclosed CAR comprises a CD28 polypeptide that comprises or has the amino acids 178 to 218 of SEQ ID NO:14.

SEQ ID NO: 14
MTLRLLFLALNFFSVQVTENKILVKQSPLLVVDSNEVSLSCRYSYNLLAKE
FRASLYKGVNSDVEVCVGNGNFTYQPQFRSNAEFNCDGDFDNETVTFRLWN
LHVNHTDIYFCKIEFMYPPPYLDNERSNGTIIHIKEKHLCHTQSSPKLFWA
LVVVAGVLFCYGLLVTVALCVIWTNSRRNRLLQSDYMNMTPRRPGLT
RKPYQPYAPARDFAAYRP

CARs comprising an intracellular signaling domain that comprises a co-stimulatory signaling region comprising 4-1BB, ICOS, or DAP 10 are disclosed in U.S. Pat. No. 7,446,190; incorporated herein by reference (i.e., a nucleotide sequence encoding 4-1BB is set forth in SEQ ID NO:15 and the protein sequence is set forth in NP_001551.2; a nucleotide sequence encoding ICOS is set forth in SEQ ID NO:16 and the protein sequence is set forth in NP_036224.1, and a nucleotide sequence encoding DAP-10 is set forth in SEQ ID NO:17 and the protein sequence is set forth in NP_055081.1), which U.S. Pat. No. 7,446,190 and Ref Protein sequences are herein incorporated by reference in their entirety.

4-1BB
SEQ ID NO: 15
ATGGGAAACAGCTGTTACAACATAGTAGCCACTCTGTTGCTGGTCCTCAAC
TTTGAGAGGACAAGATCATTGCAGGATCCTTGTAGTAACTGCCCAGCTGGT
ACATTCTGTGATAATAACAGGAATCAGATTTGCAGTCCCTGTCCTCCAAAT
AGTTTCTCCAGCGCAGGTGGACAAAGGACCTGTGACATATGCAGGCAGTGT
AAAGGTGTTTTCAGGACCAGGAAGGAGTGTTCCTCCACCAGCAATGCAGAG
TGTGACTGCACTCCAGGGTTTCACTGCCTGGGGGCAGGATGCAGCATGTGT
GAACGGATTGTAAACAAGGTCAAGAACTGACAAAAAAAGGTTGTAAAGACT
GTTGCTTTGGGACATTTAACGATCAGAAACGTGGCATCTGTCGACCCTGGA
CAAACTGTTCTTTGGATGGAAAGTCTGTGCTTGTGAATGGGACGAAGGAGA
GGGACGTGGTCTGTGGACCATCTCCAGCCGACCTCTCTCCGGGAGCATCCT
CTGTGACCCCGCCTGCCCCTGCGAGAGAGCCAGGACACTCTCCGCAGATCA
TCTCCTTCTTTCTTGCGCTGACGTCGACTGCGTTGCTCTTCCTGCTGTTCT
TCCTCACGCTCCGTTTCTCTGTTGTTAAACGGGGCAGAAAGAAACTCCTGT
ATATATTCAAACAACCATTTATGAGACCAGTACAAACTACTCAAGAGGAAG
ATGGCTGTAGCTGCCGATTTCCAGAAGAAGAAGAAGGAGGATGTGAACTGT
GA
ICOS
SEQ ID NO: 16
ATGAAGTCAGGCCTCTGGTATTTCTTTCTCTTCTGCTTGCGCATTAAAGTT
TTAACAGGAGAAATCAATGGTTCTGCCAATTATGAGATGTTTATATTTCAC
AACGGAGGTGTACAAATTTTATGCAAATATCCTGACATTGTCCAGCAATTT
AAAATGCAGTTGCTGAAAAGGGGGGCAAATACTCTGCGATCTCACTAAGAC
AAAAGGAAGTGGAAACACAGTGTCCATTAAGAGTCTGAAATTCTGCCATTC
TCAGTTATCCAACAACAGTGTCTCTTTTTTTCTACAACCTTGGACCATTCT
CATGCCAACTATTACTTCTGCAACCTATCAATTTTTGATCCTCCTCCTTTT
AAAGTAACTCTTACAGGAGGATATTTGCATATTTATGAAATCACAACTTTG
TTGCCAGCTGAAGTTCTGGTTACCCATAGGATGTGCAGCCTTTGTTGTAGT
CTGCATTTTGGGATGCATACTTATTTGTTGGCTTACAAAAAAGAAGTATTC
ATCCAGTGTGCACGACCCTAACGGTGAATACATGTTCATGAGAGCAGTGAA
CACAGCCAAAAAATCTAGACTCACAGATGTGACCCTATAA
DAP-10
SEQ ID NO: 17
ATGATCCATCTGGGTCACATCCTCTTCCTGCTTTTGCTCCCAGTGGCTGCA
GCTCAGACGACCCCAGGAGAGAGATCATCACTCCCTGCCTTTTACCCTGGC
ACTTCAGGCTCCTGTTCCGGATGTGGGTCCCTCTCTCTGCCGCTCCTGGCA
GGCCTCGTGGCTGCTGATGCGGTGGCATCGCTGCTCATCGTGGGGGCGGTG
TTCCTGTGCGCACGCCCACGCCGCAGCCCCGCCCAAGAAGATGGCAAAGTC
TACATCAACATGCCAGGCAGGGGCTGA

In certain embodiments, the intracellular signaling domain of the CAR comprises an intracellular signaling region that comprises two intracellular signaling molecules: CD28 (SEQ ID NO:10; SEQ ID NO:14) and 4-1BB or CD28 and OX40.

4-1BB can act as a tumor necrosis factor (TNF) ligand and have stimulatory activity. In certain embodiments, the 4-1BB polypeptide can have an amino acid sequence that is at least about 85%, about 90%, about 95%, about 96%, about 97%, about 98%, about 99% or about 100% identical to the sequence set forth in NCBI Reference No: P41273 (incorporated herein by reference) or NP_001552.2 (SEQ ID NO:18) or fragments thereof, and/or may optionally comprise up to one or up to two or up to three conservative amino acid substitutions.

SEQ ID NO: 18
MGNSCYNIVATLLLVLNFERTRSLQDPCSNCPAGTFCDNNRNQICSPCPPN
SFSSAGGQRTCDICRQCKGVFRTRKECSSTSNAECDCTPGFHCLGAGCSMC
EQDCKQGQELTKKGCKDCCFGTFNDQKRGICRPWTNCSLDGKSVLVNGTKE
RDVVCGPSPADLSPGASSVTPPAPAREPGHSPQIISFFLALTSTALLFLLF
FLTLRFSVVKRGRKKLLYIFKQPFMRPVQTTQEEDGCSCRFPEEEEGGCEL

In certain embodiments, an OX40 polypeptide can have an amino acid sequence that is at least about 85%, about 90%, about 95%, about 96%, about 97%, about 98%, about 99% or about 100% identical to the sequence having a NCBI Reference No: P43489 (incorporated herein by reference) or NP_003318.1 (SEQ ID NO:19), or fragments thereof, and/or may optionally comprise up to one or up to two or up to three conservative amino acid substitutions.

SEQ ID NO: 19
MCVGARRLGRGPCAALLLLGLGLSTVTGLHCVGDTYPSNDRCCHECRPGNG
MVSRCSRSQNTVCRPCGPGFYNDVVSSKPCKPCTWCNLRSGSERKQLCTAT
QDTVCRCRAGTQPLDSYKPGVDCAPCPPGHFSPGDNQACKPWTNCTLAGKH
TLQPASNSSDAICEDRDPPATQPQETQGPPARPITVQPTEAWPRTSQGPST
RPVEVPGGRAVAAILGLGLVLGLLGPLAILLALYLLRRDQRLPPDAHKPPG
GGSFRTPIQEEQADAHSTLAKI

In certain embodiments, an ICOS polypeptide can have an amino acid sequence that is at least about 85%, about 90%, about 95%, about 96%, about 97%, about 98%, about 99% or about 100% identical to the sequence having a NCBI Reference No: NP_036224.1 (SEQ ID NO:20) or fragments thereof, and/or may optionally comprise up to one or up to two or up to three conservative amino acid substitutions.

SEQ ID NO: 20
MKSGLWYFFLFCLRIKVLTGEINGSANYEMFIFHNGGVQILCKYPDIVQQF
KMQLLKGGQILCDLIKTKGSGNTVSIKSLKFCHSQLSNNSVSFFLYNLDHS
HANYYFCNLSIFDPPPFKVTLIGGYLHIYESQLCCQLKFWLPIGCAAFVVV
CILGCILICWLTKKKYSSSVHDPNGEYMFMRAVNTAKKSRLTDVTL

In certain embodiments, a DAP-12 polypeptide can have an amino acid sequence of a co-stimulatory region that is at least about 85%, about 90%, about 95%, about 96%, about 97%, about 98%, about 99% or about 100% identical to the sequence having a NCBI Reference No: NP_003323.1, NP_001166986.1, NP_001166985.1 or NP_937758.1 (each incorporated herein by reference) or fragments thereof, and/or may optionally comprise up to one or up to two or up to three conservative amino acid substitutions.

In certain embodiments, a 2B4 polypeptide can have an amino acid sequence of a co-stimulatory region that is at least about 85%, about 90%, about 95%, about 96%, about 97%, about 98%, about 99% or about 100% identical to the sequence having a NCBI Reference No: NP_057466.1, NP_001160135.1 or NP_001160136.1 (each incorporated herein by reference) or fragments thereof, and/or may optionally comprise up to one or up to two or up to three conservative amino acid substitutions.

### Cell Engineering for Expression of Non-CAR Constructs

The monocyte/macrophage cell product can be genetically modified to express non-CAR genetic constructs such as a receptor (such as a TCR, chimeric receptor, or other receptor), immune cell engager, antibody, nanobody, transcription factor, cytokine, and/or chemokine. As used herein, the term “chimeric receptor” refers to a cell surface, synthetic molecule comprising an extracellular domain, a transmembrane domain, and an intracellular signaling domain or domains from two or more molecules. As used herein, the term “immune cell engager” refers to a synthetic molecule comprising two or more binding domains designed to create an artificial immune synapse between tumor cells and immune cells for the purpose of redirecting immune effector cell function.

In certain embodiments, the genetic modification is expression of a non-modified endogenous receptor. In certain embodiments, the genetic modification is expression of a T cell receptor (TCR). In certain embodiments, a TCR consists of one alpha and one beta chain, each containing a variable domain. In certain embodiments, heterodimerization of the alpha and beta chains creates a unique receptor conformation that binds to HLA molecules displaying tumor-associated or virus-associated antigens on the surface of an antigen-presenting immune cell. In certain embodiments, the TCR binds specifically to antigens associated with tumors including but not limited to melanoma, synovial sarcoma, lung, breast, ovarian, gastric, or pancreatic. In certain embodiments, the TCR binds specifically to antigens associated with infectious diseases including but not limited to hepatitis B virus (HBV) or human immunodeficiency virus (HIV).

In certain embodiments, the genetic modification is expression of a chimeric receptor. In certain embodiments, a chimeric receptor includes an extracellular domain, a transmembrane, and one or more intracellular signaling domains. In certain embodiments, a chimeric receptor may include a linker between the extracellular domain and transmembrane domain, between the transmembrane domain and intracellular signaling domain, and/or between multiple intracellular signaling domains. In certain non-limiting embodiments, the endogenous receptor or the chimeric receptor can comprise all or a portion of molecules such as, but not limited to cytokine receptors, chemokine receptors, growth factor receptors, stimulatory molecules, costimulatory molecules, or costimulatory ligands. In certain non-limiting embodiments, the linker may be the (G4S)3 linker (SEQ ID NO: 28).

In certain embodiments, the genetic modification is expression of an immune cell engager. In certain embodiments, the immune cell engager includes one binding domain recognizing a tumor-associated antigen and one binding domain recognizing an immune cell surface protein. In certain embodiments, the immune cell engager includes more than one binding domain recognizing a tumor-associated antigen and one binding domain recognizing an immune cell surface protein. In certain embodiments, the immune cell engager includes one binding domain recognizing a tumor-associated antigen and more than one binding domain recognizing immune cell surface proteins. In certain embodiments, the binding domains of the immune cell engager are comprised of scFv domains. In certain embodiments, the immune cell engager binds specifically to tumor-associated antigens including but not limited to HER2, EpCAM, PSMA, PD-L1, CEA, EGFR, or CD33. In certain embodiments, the immune cell engager binds specifically to proteins on T cells including but not limited to CD3, CD28, and/or 4-1BB. In certain embodiments, the immune cell engager binds specifically to proteins on NK cells including but not limited to CD56, CD16, and/or NKG2D. In certain embodiments, the immune cell engager binds specifically to proteins on monocytes or macrophages including but not limited to CD64, CD40, CD80, and/or CD86.

In certain embodiments, the genetic modification is expression of an antibody or nanobody. In certain embodiments, the genetic modification is a monoclonal antibody that comprises four polypeptide chains including two identical heavy and two light chains covalently bonded by mainly disulfide interactions into a Y-shaped structure of ~150 kDa. In certain embodiments, the monoclonal antibody comprises antigen binding fragments consisting of the variable regions of the heavy and light chains, herein referred to as Fv, responsible for specific binding to an antigen. In some embodiments, the Fv region is specific for a tumor-specific antigen or tumor-associated antigen including but not limited to CD20, EGFR, HER2, PD-1, TNF-alpha, IL-1beta, IL-1RI, IL-4R, IL-6, IL-17A, or IL-23. In certain embodiments, the genetic modification is a nanobody comprised of the single variable domain of ~15 kDa derived from a heavy-chain-only antibody, responsible for specific binding to an antigen, herein referred to as VHH. In some embodiments, the VHH region is specific for a tumor-specific or tumor-associated antigen including but not limited to EGFR, EGF, HER2, PD-1, CAIX, death receptor 5 (DR5), c-Met, mesothelin, or CD33.

In certain embodiments, the genetic modification is expression of a transcription factor or transcription factor subunit. In certain embodiments, the transcription factor or transcription factor subunit modulates the function of the genetically engineered cell expressing the transcription factor. In certain embodiments, the transcription factor or transcription factor subunit can derive from, for example, the following families of transcription factors: C/EBP family, NF-kB family, STAT family, KLF family, PPAR family, AP-1 family, NFAT family, GATA family, CREB family or IRF family.

In certain embodiments, the genetic modification is expression of a cytokine. In certain embodiments, the genetic modification is expression of a chemokine. In certain embodiments, the cytokine or chemokine modulates the surrounding microenvironment. In certain embodiments, cytokine or chemokine modulates the function or activities of immune cells such as T cells, NK cells, B cells, monocytes, macrophages, or dendritic cells, including the genetically engineered cell secreting the cytokine or chemokine. In certain embodiments, the cytokine or chemokine can include but is not limited to: interleukins (such as IL-1alpha, IL-1beta, IL-2, IL-4, IL-5, IL-6, IL-7, IL-8, IL-9, IL-10, IL-11, IL-12, IL-13, IL-15, IL-17, IL-18, IL-21, IL-22), IFN-alpha, IFN-beta, IFN-gamma, IFN-lambda, TGFbeta, TNF-alpha, TNF-beta, GM-CSF, M-CSF, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, XCL1, XCL2, CX3CL1, CXCL1, CXCL2, CXLC3, CXCL4, CXCL5, CXCL6, CXCL7, CXCL8, CXCL9, CXCL10, CXCL11, CXCL12, CXCL13, CXCL14, CXCL16, or CXCL17. In some embodiments, the gene encodes a cytokine, such as human IL-15 (U.S. Pat. No. 9,931,377; SEQ ID NO:68).

IL-15
SEQ ID NO: 68
RISKPHLRSISIQCYLCLLLNSHFLTEAGIHVFILGCFSAGLPKTEANWV
NVISDLKKIEDLIQSMHIDATLYTESDVHPSCKVTAMKCFLLELQVISLE
SGDASIHDTVENLIILANNSLSSNGNVTESGCKECEELEEKNIKEFLQSF
VHIVQMFINTS

### Engineering for Expression of Multiple Genes

In some embodiments, the genetic modification is a receptor (such as a CAR, TCR, chimeric receptor, or other receptor), an immune cell engager, an antibody, a nanobody, a transcription factor, a cytokine, a chemokine, or any combination thereof. In some embodiments, multiple genes are separated by a self-cleaving peptide, including but not limited to T2A or P2A. In some embodiments, the genetic modification further includes a gene(s) encoding an additional gene product, such as a transfection marker or a suicide gene such as truncated EGFR (tEGFR) (see WO2011/056894; SEQ ID NO:67) or iCasp9 (WO2013/040371), the sequences of which are incorporated by reference herein.

tEGFR
SEQ ID NO: 67
RKVCNGIGIGEFKDSLSINATNIKHFKNCTSISGDLHILPVAFRGDSFTH
TPPLDPQELDILKTVKEITGFLLIQAWPENRTDLHAFENLEIIRGRTKQH
GQFSLAVVSLNITSLGLRSLKEISDGDVIISGNKNLCYANTINWKKLFGT
SGQKTKIISNRGENSCKATGQVCHALCSPEGCWGPEPRDCVSCRNVSRGR
ECVDKCNLLEGEPREFVENSECIQCHPECLPQAMNITCTGRGPDNCIQCA
HYIDGPHCVKTCPAGVMGENNTLVWKYADAGHVCHLCHPNCTYGCTGPGL
EGCPTNGPKIPSIATGMVGALLLLLVVALGIGLFM

### Exemplary CAR Constructs

In certain embodiments, a CAR comprises an extracellular antigen-binding domain that binds to CD19, a transmembrane domain comprising a CD28 polypeptide, and an intracellular signaling domain comprising a CD3zeta polypeptide and a co-stimulatory signaling region comprising a CD28 polypeptide. In certain embodiments, the CAR is designated 1928z. In certain embodiments, 1928z is a protein having at least about 85%, about 90%, about 95%, about 96%, about 97%, about 98%, about 99% or about 100% identical to the amino acid sequence set forth in SEQ ID NO:21. The protein sequence includes a CD8 leader sequence at amino acids 1 to 18 and is able to bind human CD19.

SEQ ID NO: 21
MALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYMQ
LSGLTSEDSAVYFCARKTISSVVDFYFDYWGQGTTVTVSSGGGGSGGGGS
GGGGSDIELTQSPKFMSTSVGDRVSVTCKASQNVGTNVAWYQQKPGQSPK
PLIYSATYRNSGVPDRFTGSGSGTDFTLTITNVQSKDLADYFCQQYNRYP
YTSGGGTKLEIKRAAAIEVMYPPPYLDNEKSNGTIIHVKGKHLCPSPLFP
GPSKPFWVLVVVGGVLACYSLLVTVAFIIFWVRSKRSRLLHSDYMNMTPR
RPGPTRKHYQPYAPPRDFAAYRSRVKFSRSAEPPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPR

In another embodiment, a CAR having an extracellular antigen-binding domain that binds to CD19, a hinge spacer from human IgG4, a transmembrane domain comprising a CD28 transmembrane domain, a 4-1BB co-stimulatory signaling region, and a CD32 intracellular signaling domain is provided. In certain embodiments, the CAR is designated 1928z1. In certain embodiments, 1928z1 is a protein having at least about 85%, about 90%, about 95%, about 96%, about 97%, about 98%, about 99% or about 100% identical to the amino acid sequence set forth in SEQ ID NO:22.

SEQ ID NO: 22
MLLLVTSLLLCELPHPAFLLIPDIQMTQTTSSLSASLGDRVTISCRASQD
ISKYLNWYQQKPDGTVKLLIYHTSRLHSGVPSRESGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSESKYGPPCPPCPMFWVLVVVGGVLACYSLLVTV
AFIIFWVKRGRKKLLYIFKQPFMRPVQTTQEEDGCSCRFPEEEEGGCELR
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPRL

In certain embodiments, a CAR comprises an extracellular antigen-binding domain that binds to MUC16, a transmembrane domain comprising a CD28 polypeptide, and an intracellular signaling domain comprising a CD3zeta polypeptide and a co-stimulatory signaling region comprising a CD28 polypeptide. In certain embodiments, the CAR is designated 4H1128z. In certain embodiments, 4H1128z is a protein having at least about 85%, about 90%, about 95%, about 96%, about 97%, about 98%, about 99% or about 100% identical to the amino acid sequence set forth in SEQ ID NO:23. The protein includes a CD8 leader sequence at amino acids 1 to 18 and binds to the MUC-16 ectodomain.

SEQ ID NO: 23
MALPVTALLLPLALLLHAEVKLQESGGGFVKPGGSLKVSCAASGFTFSSY
AMSWVRLSPEMRLEWVATISSAGGYIFYSDSVQGRFTISRDNAKNTLHLQ
MGSLRSGDTAMYYCARQGFGNYGDYYAMDYWGQGTTVTVSSGGGGSGGGG
SGGGGSDIELTQSPSSLAVSAGEKVTMSCKSSQSLLNSRTRKNQLAWYQQ
KPGQSPELLIYWASTRQSGVPDRFTGSGSGTDFTLTISSVQAEDLAVYYC
QQSYNLLTFGPGTKLEIKRAAAIEVMYPPPYLDNEKSNGTIIHVKGKHLC
PSPLFPGPSKPFWVLVVVGGVLACYSLLVTVAFIIFWVRSKRSRLLHSDY
MNMTPRRPGPTRKHYQPYAPPRDFAAYRSRVKFSRSAEPPAYQQGQNQLY
NELNLGRREEYDVLDKRRGRDPEMGGKPRRKNPQEGLYNELQKDKMAEAY
SEIGMKGERRRGKGHDGLYQGLSTATKDTYDALHMQALPPR

In certain embodiments, a CAR comprises an extracellular antigen-binding domain that binds to CD19, a transmembrane domain comprising a CD8 polypeptide, and an intracellular signaling domain comprising a CD3zeta polypeptide and a co-stimulatory signaling region comprising a 4-1BB polypeptide. In certain embodiments, the CAR is 19BBz. An exemplary protein sequence of the 19BBz polypeptide is set forth in SEQ ID NO: 24.

SEQ ID NO: 24
MALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYMQ
LSGLTSEDSAVYFCARKTISSVVDFYFDYWGQGTTVTVSSGGGGSGGGGS
GGGGSDIELTQSPKFMSTSVGDRVSVTCKASQNVGTNVAWYQQKPGQSPK
PLIYSATYRNSGVPDRFTGSGSGTDFTLTITNVQSKDLADYFCQQYNRYP
YTSGGGTKLEIKRAAAPTTTPAPRPPTPAPTIASQPLSLRPEACRPAAGG
AVHTRGLDFACDIYIWAPLAGTCGVLLLSLVITLYCNKRGRKKLLYIFKQ
PFMRPVQTTQEEDGCSCRFPEEEEGGCELRVKFSRSAEPPAYQQGQNQLY
NELNLGRREEYDVLDKRRGRDPEMGGKPRRKNPQEGLYNELQKDKMAEAY
SEIGMKGERRRGKGHDGLYQGLSTATKDTYDALHMQALPPR

In certain embodiments, a CAR having an extracellular antigen-binding domain that binds to CD19, a hinge spacer from human IgG4, a transmembrane domain comprising a CD28 transmembrane domain, a 4-1BB co-stimulatory signaling region, and a CD3zeta intracellular signaling domain is provided. An exemplary CD19 CAR protein sequence, that also includes a truncated EGFR (tEGFR) polypeptide attached to the carboxy terminal portion of the CAR by a self-cleaving 2A peptide, is set forth in SEQ ID NO:61.

SEQ ID NO: 61
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSESKYGPPCPPCPMFWVLVVVGGVLACYSLLVTV
AFIIFWVKRGRKKLLYIFKQPFMRPVQTTQEEDGCSCRFPEEEEGGCELR
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPRLEGGGEGRGSLLTCGDVEENPGPRMLLLVTSLLLCELPH
PAFLLIPRKVCNGIGIGEFKDSLSINATNIKHFKNCTSISGDLHILPVAF
RGDSFTHTPPLDPQELDILKTVKEITGFLLIQAWPENRTDLHAFENLEII
RGRTKQHGQFSLAVVSLNITSLGLRSLKEISDGDVIISGNKNLCYANTIN
WKKLFGTSGQKTKIISNRGENSCKATGQVCHALCSPEGCWGPEPRDCVSC
RNVSRGRECVDKCNLLEGEPREFVENSECIQCHPECLPQAMNITCTGRGP
DNCIQCAHYIDGPHCVKTCPAGVMGENNTLVWKYADAGHVCHLCHPNCTY
GCTGPGLEGCPTNGPKIPSIATGMVGALLLLLVVALGIGLFM

In certain embodiments, a CAR having an extracellular antigen-binding domain that binds to CD19, a hinge spacer from human IgG4, a transmembrane domain of a CD28 transmembrane domain, a 4-1BB co-stimulatory signaling region, and a CD3ζ intracellular signaling domain is provided. An exemplary CD19 CAR protein sequence, that also includes a human IL-15 attached to the carboxy terminal portion of the CAR by a self-cleaving 2A peptide, is set forth in SEQ ID NO:62.

SEQ ID NO: 62
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSESKYGPPCPPCPMFWVLVVVGGVLACYSLLVTV
AFIIFWVKRGRKKLLYIFKQPFMRPVQTTQEEDGCSCRFPEEEEGGCELR
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPRLEGGGEGRGSLLTCGDVEENPGPRMRISKPHLRSISIQC
YLCLLLNSHFLTEAGIHVFILGCFSAGLPKTEANWVNVISDLKKIEDLIQ
SMHIDATLYTESDVHPSCKVTAMKCFLLELQVISLESGDASIHDTVENLI
ILANNSLSSNGNVTESGCKECEELEEKNIKEFLQSFVHIVQMFINTS

The corresponding nucleic acid sequence that can encode the construct in SEQ ID NO: 62 is set forth in SEQ ID NO:69.

SEQ ID NO: 69
ATGCTGCTGCTGGTGACCAGCCTGCTGCTGTGCGAGCTGCCCCACCCCGC
CTTTCGCTGATCCCCGACATCCAGATGACCCAGACCACCTCCAGCCTGAG
CGCCAGCCTGGGCGACCGGGTGACCATCAGCTGCCGGGCCAGCCAGGACA
TCAGCAAGTACCTGAACTGGTATCAGCAGAAGCCCGACGGCACCGTCAAG
CTGCTGATCTACCACACCAGCCGGCTGCACAGCGGCGTGCCCAGCCGGTT
TAGCGGCAGCGGCTCCGGCACCGACTACAGCCTGACCATCTCCAACCTGG
AACAGGAAGATATCGCCACCTACTTTTGCCAGCAGGGCAACACACTGCCC
TACACCTTTGGCGGCGGAACAAAGCTGGAAATCACCGGCAGCACCTCCGG
CAGCGGCAAGCCTGGCAGCGGCGAGGGCAGCACCAAGGGCGAGGTGAAGC
TGCAGGAAAGCGGCCCTGGCCTGGTGGCCCCCAGCCAGAGCCTGAGCGTG
ACCTGCACCGTGAGCGGCGTGAGCCTGCCCGACTACGGCGTGAGCTGGAT
CCGGCAGCCCCCCAGGAAGGGCCTGGAATGGCTGGGCGTGATCTGGGGCA
GCGAGACCACCTACTACAACAGCGCCCTGAAGAGCCGGCTGACCATCATC
AAGGACAACAGCAAGAGCCAGGTGTTCCTGAAGATGAACAGCCTGCAGAC
CGACGACACCGCCATCTACTACTGCGCCAAGCACTACTACTACGGCGGCA
GCTACGCCATGGACTACTGGGGCCAGGGCACCAGCGTGACCGTGAGCAGC
GAATCTAAGTACGGACCGCCCTGCCCCCCTTGCCCTATGTTCTGGGTGCT
GGTGGTGGTCGGAGGCGTGCTGGCCTGCTACAGCCTGCTGGTCACCGTGG
CCTTCATCATCTTTTGGGTGAAACGGGGCAGAAAGAAACTCCTGTATATA
TTCAAACAACCATTTATGAGACCAGTACAAACTACTCAAGAGGAAGATGG
CTGTAGCTGCCGATTTCCAGAAGAAGAAGAAGGAGGATGTGAACTGAGGG
TGAAGTTCAGCAGAAGCGCCGACGCCCCTGCCTACCAGCAGGGCCAGAAT
CAGCTGTACAACGAGCTGAACCTGGGCAGAAGGGAAGAGTACGACGTCCT
GGATAAGCGGAGAGGCCGGGACCCTGAGATGGGCGGCAAGCCTCGGCGGA
AGAACCCCCAGGAAGGCCTGTATAACGAACTGCAGAAAGACAAGATGGCC
GAGGCCTACAGCGAGATCGGCATGAAGGGCGAGCGGAGGCGGGGCAAGGG
CCACGACGGCCTGTATCAGGGCCTGTCCACCGCCACCAAGGATACCTACG
ACGCCCTGCACATGCAGGCCCTGCCCCCAAGGCTCGAGGGCGGCGGAGAG
GGCAGAGGAAGTCTTCTAACATGCGGTGACGTGGAGGAGAATCCCGGCCC
TAGGATGCGGATTTCCAAACCTCACCTGCGCTCTATCTCTATCCAGTGCT
ATCTGTGCCTGCTGCTGAACTCACATTTCCTGACCGAAGCCGGCATCCAC
GTGTTCATCCTGGGCTGCTTTTCCGCCGGCCTGCCAAAGACCGAGGCAAA
CTGGGTGAATGTGATCTCTGACCTGAAGAAGATCGAGGATCTGATCCAGA
GCATGCACATCGACGCCACCCTGTACACAGAGTCCGATGTGCACCCTTCT
TGCAAGGTGACAGCCATGAAGTGTTTCCTGCTGGAGCTGCAGGTCATCAG
CCTGGAGAGCGGCGACGCCTCTATCCACGATACCGTGGAGAACCTGATCA
TCCTGGCCAACAATAGCCTGAGCAGCAACGGCAATGTGACAGAGTCCGGC
TGCAAGGAGTGTGAGGAGCTGGAGGAGAAGAATATCAAAGAGTTCCTGCA
GTCATTCGTCCATATCGTCCAGATGTTTATCAATACCTCCTAA

In certain embodiments, a CAR having an extracellular antigen-binding domain that binds to CD19, a hinge spacer from human IgG4, a transmembrane domain of a CD28 transmembrane domain, a 2B4 co-stimulatory signaling region, and a CD3zeta intracellular signaling domain is provided. An exemplary CD19 CAR protein sequence, that also includes a human IL-15 attached to the carboxy terminal portion of the CAR by a self-cleaving 2A peptide, is set forth in SEQ ID NO:70.

SEQ ID NO: 70
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSESKYGPPCPPCPMFWVLVVVGGVLACYSLLVTV
AFIIFWVWRRKRKEKQSETSPKEFLTIYEDVKDLKTRRNHEQEQTFPGGG
STIYSMIQSQSSAPTSQEPAYTLYSLIQPSRKSGSRKRNHSPSFNSTIYE
VIGKSQPKAQNPARLSRKELENFDVYSRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPRLEGGGEGRGSL
LTCGDVEENPGPRMRISKPHLRSISIQCYLCLLLNSHFLTEAGIHVFILG
CFSAGLPKTEANWVNVISDLKKIEDLIQSMHIDATLYTESDVHPSCKVTA
MKCFLLELQVISLESGDASIHDTVENLIILANNSLSSNGNVTESGCKECE
ELEEKNIKEFLQSFVHIVQMFINTS

The corresponding nucleic acid sequence encoding the SEQ ID NO:70 construct is set forth in SEQ ID NO:71.

SEQ ID NO: 71
ATGCTGCTGCTGGTGACCAGCCTGCTGCTGTGCGAGCTGCCCCACCCCGC
CTTTCTGCTGATCCCCGACATCCAGATGACCCAGACCACCTCCAGCCTGA
GCGCCAGCCTGGGCGACCGGGTGACCATCAGCTGCCGGGCCAGCCAGGAC
ATCAGCAAGTACCTGAACTGGTATCAGCAGAAGCCCGACGGCACCGTCAA
GCTGCTGATCTACCACACCAGCCGGCTGCACAGCGGCGTGCCCAGCCGGT
TTAGCGGCAGCGGCTCCGGCACCGACTACAGCCTGACCATCTCCAACCTG
GAACAGGAAGATATCGCCACCTACTTTTGCCAGCAGGGCAACACACTGCC
CTACACCTTTGGCGGCGGAACAAAGCTGGAAATCACCGGCAGCACCTCCG
GCAGCGGCAAGCCTGGCAGCGGCGAGGGCAGCACCAAGGGCGAGGTGAAG
CTGCAGGAAAGCGGCCCTGGCCTGGTGGCCCCCAGCCAGAGCCTGAGCGT
GACCTGCACCGTGAGCGGCGTGAGCCTGCCCGACTACGGCGTGAGCTGGA
TCCGGCAGCCCCCCAGGAAGGGCCTGGAATGGCTGGGCGTGATCTGGGGC
AGCGAGACCACCTACTACAACAGCGCCCTGAAGAGCCGGCTGACCATCAT
CAAGGACAACAGCAAGAGCCAGGTGTTCCTGAAGATGAACAGCCTGCAGA
CCGACGACACCGCCATCTACTACTGCGCCAAGCACTACTACTACGGCGGC
AGCTACGCCATGGACTACTGGGGCCAGGGCACCAGCGTGACCGTGAGCAG
CGAATCTAAGTACGGACCGCCCTGCCCCCCTTGCCCTATGTTCTGGGTGC
TGGTGGTGGTCGGAGGCGTGCTGGCCTGCTACAGCCTGCTGGTCACCGTG
GCCTTCATCATCTTTTGGGTGTGGAGGAGGAAGAGGAAGGAGAAGCAGAG
CGAGACAAGCCCTAAGGAGTTTCTGACAATCTATGAAGACGTGAAGGACC
TGAAGACACGGAGAAACCACGAGCAGGAGCAGACCTTCCCTGGAGGAGGC
AGCACAATCTACTCCATGATCCAGTCTCAGAGCAGCGCCCCCACCTCCCA
GGAGCCTGCCTACACACTGTATAGCCTGATCCAGCCATCCCGGAAGTCTG
GCAGCAGGAAGCGCAACCACTCCCCCTCTTTTAATTCTACCATCTATGAA
GTGATCGGCAAGAGCCAGCCCAAGGCACAGAACCCCGCACGACTGAGCAG
GAAGGAACTGGAGAACTTTGATGTCTACTCTAGGGTGAAGTTCAGCAGAA
GCGCCGACGCCCCTGCCTACCAGCAGGGCCAGAATCAGCTGTACAACGAG
CTGAACCTGGGCAGAAGGGAAGAGTACGACGTCCTGGATAAGCGGAGAGG
CCGGGACCCTGAGATGGGCGGCAAGCCTCGGCGGAAGAACCCCCAGGAAG
GCCTGTATAACGAACTGCAGAAAGACAAGATGGCCGAGGCCTACAGCGAG
ATCGGCATGAAGGGCGAGCGGAGGCGGGGCAAGGGCCACGACGGCCTGTA
TCAGGGCCTGTCCACCGCCACCAAGGATACCTACGACGCCCTGCACATGC
AGGCCCTGCCCCCAAGGCTCGAGGGCGGCGGAGAGGGCAGAGGAAGTCTT
CTAACATGCGGTGACGTGGAGGAGAATCCCGGCCCTAGGATGCGGATTTC
CAAACCTCACCTGCGCTCTATCTCTATCCAGTGCTATCTGTGCCTGCTGC
TGAACTCACATTTCCTGACCGAAGCCGGCATCCACGTGTTCATCCTGGGC
TGCTTTTCCGCCGGCCTGCCAAAGACCGAGGCAAACTGGGTGAATGTGAT
CTCTGACCTGAAGAAGATCGAGGATCTGATCCAGAGCATGCACATCGACG
CCACCCTGTACACAGAGTCCGATGTGCACCCTTCTTGCAAGGTGACAGCC
ATGAAGTGTTTCCTGCTGGAGCTGCAGGTCATCAGCCTGGAGAGCGGCGA
CGCCTCTATCCACGATACCGTGGAGAACCTGATCATCCTGGCCAACAATA
GCCTGAGCAGCAACGGCAATGTGACAGAGTCCGGCTGCAAGGAGTGTGAG
GAGCTGGAGGAGAAGAATATCAAAGAGTTCCTGCAGTCATTCGTCCATAT
CGTCCAGATGTTTATCAATACCTCCTAA

In certain embodiments, a CAR having an extracellular antigen-binding domain that binds to CD19, a hinge spacer from human IgG4, a transmembrane domain of a NKG2D transmembrane domain, a 4-1BB co-stimulatory signaling region, and a CD3zeta intracellular signaling domain is provided. Use of the NKG2Ds transmembrane domain has been used previously (Xu et al., J. Hematol. Oncol. 12:49, 2019) although some suggest that because NKGD2 is a type II membrane protein it should be inserted in the opposite orientation. An exemplary CD19 CAR protein sequence, that also includes a human IL-15 attached to the carboxy terminal portion of the CAR by a self-cleaving 2A peptide, is set forth in SEQ ID NO:72.

SEQ ID NO: 72
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSESKYGPPCPPCPPFFFCCFIAVAMGIRFIIMVT
KRGRKKLLYIFKQPFMRPVQTTQEEDGCSCRFPEEEEGGCELRVKFSRSA
DAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQA
LPRLEGGGEGRGSLLTCGDVEENPGPRMRISKPHLRSISIQCYLCLLLNS
HFLTEAGIHVFILGCFSAGLPKTEANWVNVISDLKKIEDLIQSMHIDATL
YTESDVHPSCKVTAMKCFLLELQVISLESGDASIHDTVENLIILANNSLS
SNGNVTESGCKECEELEEKNIKEFLQSFVHIVQMFINTS

The corresponding nucleic acid sequence encoding the SEQ ID NO:72 construct is set forth in SEQ ID NO:73.

SEQ ID NO: 73
ATGCTGCTGCTGGTGACCAGCCTGCTGCTGTGCGAGCTGCCCCACCCCGC
CTTTCTGCTGATCCCCGACATCCAGATGACCCAGACCACCTCCAGCCTGA
GCGCCAGCCTGGGCGACCGGGTGACCATCAGCTGCCGGGCCAGCCAGGAC
ATCAGCAAGTACCTGAACTGGTATCAGCAGAAGCCCGACGGCACCGTCAA
GCTGCTGATCTACCACACCAGCCGGCTGCACAGCGGCGTGCCCAGCCGGT
TTAGCGGCAGCGGCTCCGGCACCGACTACAGCCTGACCATCTCCAACCTG
GAACAGGAAGATATCGCCACCTACTTTTGCCAGCAGGGCAACACACTGCC
CTACACCTTTGGCGGCGGAACAAAGCTGGAAATCACCGGCAGCACCTCCG
GCAGCGGCAAGCCTGGCAGCGGCGAGGGCAGCACCAAGGGCGAGGTGAAG
CTGCAGGAAAGCGGCCCTGGCCTGGTGGCCCCCAGCCAGAGCCTGAGCGT
GACCTGCACCGTGAGCGGCGTGAGCCTGCCCGACTACGGCGTGAGCTGGA
TCCGGCAGCCCCCCAGGAAGGGCCTGGAATGGCTGGGCGTGATCTGGGGC
AGCGAGACCACCTACTACAACAGCGCCCTGAAGAGCCGGCTGACCATCAT
CAAGGACAACAGCAAGAGCCAGGTGTTCCTGAAGATGAACAGCCTGCAGA
CCGACGACACCGCCATCTACTACTGCGCCAAGCACTACTACTACGGCGGC
AGCTACGCCATGGACTACTGGGGCCAGGGCACCAGCGTGACCGTGAGCAG
CGAATCTAAGTACGGACCGCCCTGCCCCCCTTGCCCTCCCTTCTTTTTCT
GCTGTTTTATCGCCGTGGCTATGGGCATCCGGTTCATCATCATGGTGACC
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTGAGGGTGAAGTTCAGCAGAAGCGCC
GACGCCCCTGCCTACCAGCAGGGCCAGAATCAGCTGTACAACGAGCTGAA
CCTGGGCAGAAGGGAAGAGTACGACGTCCTGGATAAGCGGAGAGGCCGGG
ACCCTGAGATGGGCGGCAAGCCTCGGCGGAAGAACCCCCAGGAAGGCCTG
TATAACGAACTGCAGAAAGACAAGATGGCCGAGGCCTACAGCGAGATCGG
CATGAAGGGCGAGCGGAGGCGGGGCAAGGGCCACGACGGCCTGTATCAGG
GCCTGTCCACCGCCACCAAGGATACCTACGACGCCCTGCACATGCAGGCC
CTGCCCCCAAGGCTCGAGGGCGGCGGAGAGGGCAGAGGAAGTCTTCTAAC
ATGCGGTGACGTGGAGGAGAATCCCGGCCCTAGGATGCGGATTTCCAAAC
CTCACCTGCGCTCTATCTCTATCCAGTGCTATCTGTGCCTGCTGCTGAAC
TCACATTTCCTGACCGAAGCCGGCATCCACGTGTTCATCCTGGGCTGCTT
TCGAGGATCTGATCCAGAGCATGCACATCGACGCCACCCTGTACACAGAG
TTCCGCCGGCCTGCCAAAGACCGAGGCAAACTGGGTGAATGTGATCTCTG
ACCTGAAGAAGATCCGATGTGCACCCTTCTTGCAAGGTGACAGCCATGAA
GTGTTTCCTGCTGGAGCTGCAGGTCATCAGCCTGGAGAGCGGCGACGCCT
CTATCCACGATACCGTGGAGAACCTGATCATCCTGGCCAACAATAGCCTG
AGCAGCAACGGCAATGTGACAGAGTCCGGCTGCAAGGAGTGTGAGGAGCT
GGAGGAGAAGAATATCAAAGAGTTCCTGCAGTCATTCGTCCATATCGTCC
AGATGTTTATCAATACCTCCTAA

In certain embodiments, a CAR having an extracellular antigen-binding domain that binds to CD19, a hinge spacer from human IgG₄, a transmembrane domain of a NKG2D transmembrane domain, a 2B4 co-stimulatory signaling region, and a CD3ζ intracellular signaling domain is provided. An exemplary CD19 CAR protein sequence, that also includes a human IL-15 attached to the carboxy terminal portion of the CAR by a self-cleaving 2A peptide, is set forth in SEQ ID NO:74.

SEQ ID NO: 74
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSESKYGPPCPPCPPFFFCCFIAVAMGIRFIIMVT
WRRKRKEKQSETSPKEFLTIYEDVKDLKTRRNHEQEQTFPGGGSTIYSMI
QSQSSAPTSQEPAYTLYSLIQPSRKSGSRKRNHSPSFNSTIYEVIGKSQP
KAQNPARLSRKELENFDVYSRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPRLEGGGEGRGSLLTCGDVE
ENPGPRMRISKPHLRSISIQCYLCLLLNSHFLTEAGIHVFILGCFSAGLP
KTEANWVNVISDLKKIEDLIQSMHIDATLYTESDVHPSCKVTAMKCFLLE
LQVISLESGDASIHDTVENLIILANNSLSSNGNVTESGCKECEELEEKNI
KEFLQSFVHIVQMFINTS

The corresponding nucleic acid sequence encoding the SEQ ID NO:74 construct is set forth in SEQ ID NO:75.

SEQ ID NO: 75
atgctgctgctggtgaccagcctgctgctgtgcgagctgccccaccccgc
ctttctgctgatccccgacatccagatgacccagaccacctccagcctga
gcgccagcctgggcgaccgggtgaccatcagctgccgggccagccaggac
atcagcaagtacctgaactggtatcagcagaagcccgacggcaccgtcaa
gctgctgatctaccacaccagccggctgcacagcggcgtgcccagccggt
ttagcggcagcggctccggcaccgactacagcctgaccatctccaacctg
gaacaggaagatatcgccacctacttttgccagcagggcaacacactgcc
ctacacctttggcggcggaacaaagctggaaatcaccggcagcacctccg
gcageggcaagcctggcagcggcgagggcagcaccaagggcgaggtgaag
ctgcaggaaagcggccctggcctggtggcccccagccagagcctgagcgt
gacctgcaccgtgagcggcgtgagcctgcccgactacggcgtgagctgga
tccggcagccccccaggaagggcctggaatggctgggcgtgatctggggc
agcgagaccacctactacaacagcgccctgaagagccggctgaccatcat
caaggacaacagcaagagccaggtgttcctgaagatgaacagcctgcaga
ccgacgacaccgccatctactactgcgccaagcactactactacggcggc
agctacgccatggactactggggccagggcaccagcgtgaccgtgagcag
cGaatctaagtacggaccgccctgccccccttgccctCCCTTCTTTTTCT
GCTGTTTTATCGCCGTGGCTATGGGCATCCGGTTCATCATCATGGTGACC
TGGAGGAGGAAGAGGAAGGAGAAGCAGAGCGAGACAAGCCCTAAGGAGTT
TCTGACAATCTATGAAGACGTGAAGGACCTGAAGACACGGAGAAACCACG
AGCAGGAGCAGACCTTCCCTGGAGGAGGCAGCACAATCTACTCCATGATC
CAGTCTCAGAGCAGCGCCCCCACCTCCCAGGAGCCTGCCTACACACTGTA
TAGCCTGATCCAGCCATCCCGGAAGTCTGGCAGCAGGAAGCGCAACCACT
CCCCCTCTTTTAATTCTACCATCTATGAAGTGATCGGCAAGAGCCAGCCC
AAGGCACAGAACCCCGCACGACTGAGCAGGAAGGAACTGGAGAACTTTGA
TGTCTACTCTAgggtgaagttcagcagaagcgccgacgcccctgcctacc
agcagggccagaatcagctgtacaacgagctgaacctgggcagaagggaa
gagtacgacgtcctggataagcggagaggccgggaccctgagatgggcgg
caagcctcggcggaagaacccccaggaaggcctgtataacgaactgcaga
aagacaagatggccgaggcctacagcgagatcggcatgaagggcgagcgg
aggcggggcaagggccacgacggcctgtatcagggcctgtccaccgccac
caaggatacctacgacgccctgcacatgcaggccctgcccccaaggCtcg
agggggcggagagggcagaggaagtcttctaacatgcggtgacgtggagg
agaatcccggccctAggATGCGGATTTCCAAACCTCACCTGCGCTCTATC
TCTATCCAGTGCTATCTGTGCCTGCTGCTGAACTCACATTTCCTGACCGA
AGCCGGCATCCACGTGTTCATCCTGGGCTGCTTTTCCGCCGGCCTGCCAA
AGACCGAGGCAAACTGGGTGAATGTGATCTCTGACCTGAAGAAGATCGAG
GATCTGATCCAGAGCATGCACATCGACGCCACCCTGTACACAGAGTCCGA
TGTGCACCCTTCTTGCAAGGTGACAGCCATGAAGTGTTTCCTGCTGGAGC
TGCAGGTCATCAGCCTGGAGAGCGGCGACGCCTCTATCCACGATACCGTG
GAGAACCTGATCATCCTGGCCAACAATAGCCTGAGCAGCAACGGCAATGT
GACAGAGTCCGGCTGCAAGGAGTGTGAGGAGCTGGAGGAGAAGAATATCA
AAGAGTTCCTGCAGTCATTCGTCCATATCGTCCAGATGTTTATCAATACC
TCCTAA

### Expression Constructs

In certain embodiments, a receptor (such as a CAR, TCR, chimeric receptor, or other receptor), an immune engager, an antibody, a nanobody, a transcription factor, a cytokine, and/or a chemokine can further be expressed from a nucleic acid comprising an inducible promoter, for expressing nucleic acid sequences in human cells. In certain embodiments, the inducible promoter is responsive to factors in the tumor microenvironment. In certain embodiments, the inducible promoter is responsive to exogenously administered molecules. In certain embodiments, a receptor (such as a CAR, TCR, chimeric receptor, or other receptor), an immune cell engager, an antibody, a nanobody, a transcription factor, a cytokine, and/or a chemokine can further be expressed from a nucleic acid comprising a constitutive promoter, such as promoters for ubiquitin C (UbiC), PGK, EF-1alpha, MND (a synthetic viral promoter that contains the U3 region of a modified Moloney murine leukemia virus long terminal repeat with myeloproliferative sarcoma virus enhancer), or Chicken beta actin.

Methods of preparing genetically modified monocytes/macrophage compositions and/or preparations for immunotherapy comprise introducing into the CD11b⁺ HLA-DR⁺ (mainly monocyte/macrophage) cells a polynucleotide(s) encoding a receptor (such as a CAR, TCR, chimeric receptor, or other receptor), an immune cell engager, an antibody, a nanobody, a transcription factor, a cytokine, and/or a chemokine) into the cells. The polynucleotide(s) encoding the desired molecule can be introduced into the HSPCs, before, during, or after expansion, or into HSPCs or monocyte/macrophage compositions and/or preparations before, during, or after differentiation. Some embodiments relate to a method of engineering a monocyte/macrophage composition and/or preparation by transforming, transducing, or transfecting an HSPC or monocyte/macrophage composition and/or preparation with at least one polynucleotide encoding a receptor (such as a CAR, a TCR, a chimeric receptor, or other receptor), an immune cell engager, an antibody, a nanobody, a transcription factor, a cytokine, and/or a chemokine, and expressing the polynucleotide in the cell. Desired polynucleotides encoding genes can be introduced into HSPCs or other cells by any method known in the art, including transfection, electroporation, microinjection, lipofection, calcium phosphate mediated transfection, infection with a viral or bacteriophage vector containing the gene sequences, cell fusion, chromosome-mediated gene transfer, microcell-mediated gene transfer, spheroplast fusion, using CRISPR or other rare-cutting endonuclease (e.g., TALE-nuclease or Cas9 endonuclease), and the like. Numerous techniques are known in the art for the introduction of foreign genes into cells (see e.g., Loeffler and Behr, Meth. Enzymol. 217:599-618, 1993; Cohen et al., Meth. Enzymol. 217:618-644, 1993; Cline, Pharmac. Ther. 29:69-92, 1985) and can be used, provided that the necessary physiological functions of the recipient cells are not disrupted. The technique should provide for the stable transfer of the gene to the cell, so that the gene is expressible by the cell and preferably heritable and expressible by its cell progeny. In some embodiments, the method of transfer includes the transfer of a selectable marker or tag sequence to the cells. The cells are then placed under selection to isolate those cells that have taken up and are expressing the transferred gene. In a preferred embodiment, the polynucleotide(s) or genes are included in lentiviral vectors in view of being stably expressed in the cells.

In an embodiment, a receptor (such as a CAR, a TCR, a chimeric receptor, or other receptor), an immune cell engager, an antibody, a nanobody, a transcription factor, a cytokine, and/or a chemokine is introduced into HSPCs during the expansion phase and prior to differentiation, to form a receptor-(such as a CAR, TCR, chimeric receptor, or other receptor), an immune cell engager-, an antibody-, a nanobody-, a transcription factor-, a cytokine-, and/or a chemokine-expressing HSPCs and/or a receptor-(such as a CAR, TCR, chimeric receptor, or other receptor), an immune cell engager-, an antibody-, a nanobody-, a transcription factor-, a cytokine-, and/or a chemokine-expressing monocyte/macrophage. In a preferred embodiment, the receptor-(such as a CAR, TCR, chimeric receptor, or other receptor), immune cell engager-, antibody-, nanobody-, transcription factor-, cytokine-, and/or chemokine is introduced into HSPCs during the expansion phase and prior to differentiation, to form receptor-(such as a CAR, TCR, chimeric receptor, or other receptor), immune cell engager-, antibody-, nanobody-, transcription factor-, cytokine-, and/or chemokine-expressing HSPCs and/or monocyte/macrophage. In another embodiment, a receptor-(such as a CAR, TCR, chimeric receptor, or other receptor), an immune cell engager-, an antibody-, a nanobody-, a transcription factor-, a cytokine-, and/or a chemokine is introduced into CD11b⁺ HLA-DR⁺ monocyte/macrophage after expansion and during the differentiation phase, to form a receptor-(such as a CAR, a TCR, a chimeric receptor, or other receptor), an immune cell engager-, an antibody-, a nanobody-, a transcription factor-, a cytokine-, and/or a chemokine-expressing HSPCs and/or a receptor-(such as a CAR, a TCR, a chimeric receptor, or other receptor), an immune cell engager-, an antibody-, a nanobody-, a transcription factor-, a cytokine-, and/or a chemokine-expressing monocyte/macrophage. In a preferred embodiment, the receptor (such as a CAR, TCR, chimeric receptor, or other receptor), immune cell engager, antibody, nanobody, transcription factor, cytokine, and/or chemokine is introduced into CD11b⁺ HLA-DR⁺ monocyte/macrophage after expansion and during the differentiation phase, to form a receptor-(such as a CAR, TCR, chimeric receptor, or other receptor), an immune cell engager-, an antibody-, a nanobody-, a transcription factor-, a cytokine-, and/or a chemokine-HSPCs and/or a receptor-(such as a CAR, TCR, chimeric receptor, or other receptor), an immune cell engager-, an antibody-, a nanobody-, a transcription factor-, a cytokine-, and/or a chemokine-expressing-monocyte/macrophage cells. The receptor (such as a CAR, TCR, chimeric receptor, or other receptor), immune cell engager, antibody, nanobody, transcription factor, cytokine, and/or chemokine introduction, cell expansion, and cell differentiation mentioned above are each carried out as described above and below.

The different methods described above involve introducing a receptor (such as a CAR, TCR, chimeric receptor, or other receptor), an immune cell engager, an antibody, a nanobody, a transcription factor, a cytokine, and/or a chemokine into a cell. As a non-limiting example, a receptor (such as a CAR, TCR, chimeric receptor, or other receptor), an immune cell engager, an antibody, a nanobody, a transcription factor, a cytokine, and/or a chemokine can be introduced as a transgene(s) encoded by one or more than one plasmid vector. The plasmid vector can also contain a selection marker which provides for identification and/or selection of cells which received said vector.

Polypeptides, such as a receptor (such as a CAR, a TCR, a chimeric receptor, or other receptor), an immune cell engager, an antibody, a nanobody, a transcription factor, a cytokine, and/or a chemokine, can be synthesized in situ in the cell as a result of the introduction of polynucleotides encoding the polypeptides into the cell.

Methods for viral mediated introduction of a polynucleotide construct into cells are known in the art, and include as non-limiting examples recombinant viral vectors (e.g., lentiviruses, retroviruses, adenoviruses, and AAVs).

### Oncolytic Viruses

An oncolytic virus can be used as a form of immunotherapy in which the virus selectively replicates within and lyses tumor cells, leading to activation of innate immune cells including, but not limited to, monocytes, macrophages, dendritic cells, or NK cells. Viral oncolysis of tumor cells results in dispersal of viral progeny and antigen release. An innate function of macrophages is phagocytic activity to clear virus particles and virally infected cells. Macrophage phagocytic activity can result in immune activation including, but not limited to, release of cytokines, a shift in tumor-resident macrophages from a tumor-supportive phenotype towards an anti-tumor pro-inflammatory phenotype, sustained inflammation, recruitment of immune cells to infiltrate the suppressive tumor microenvironment (TME), or macrophage-mediated antigen cross-presentation to T cells to initiate a cascade of adaptive immune responses against the tumor cells.

In certain embodiments, an oncolytic virus comprises modified herpes simplex virus, adenovirus, vaccinia virus, myxoma virus, measles virus, Coxsackievirus, parvovirus, or human intestinal cytopathic orphan virus. In some embodiments, oncolytic virus is delivered through systemic administration, intratumoral injection, or via carrier cells pre-loaded with virus ex vivo prior to administration. In some embodiments, carrier cells comprise NK cells, monocytes, dendritic cells, and/or macrophages. In some embodiments, carrier cells comprise the monocyte/macrophage product.

Cryopreservation of the Monocyte/Macrophage Composition and/or Preparation

A monocyte/macrophage composition and/or preparation, with or without genetic engineering, can be divided and frozen in one or more bags (or units). In a preferred embodiment, from about 50 to about 500 million total cells are frozen in a single bag (or unit). In another preferred embodiment, from about 100 to about 500 million cells are frozen in a single bag (or unit). In other preferred embodiments, about 50, 100, 200, 300, 400 or 400 million cells are frozen in a single bag (or unit). In some embodiments a single bag (or unit) contains about 50 million to about 2 billion viable cells per dose. In some embodiments, a single bag (or unit) contains about 100 million, about 200 million, about 300 million, about 400 million, about 500 million, about 600 million, about 750 million, about 1 billion, about 1.5 billion or about 2 billion viable cells. In some embodiments, a single bag contains about 50 million to about 2 billion viable CD11b⁺ HLA-DR⁺ monocyte/macrophage per dose. In some embodiments, a single bag (or unit) contains about 100 million, about 200 million, about 300 million, about 400 million, about 500 million, about 600 million, about 750 million, about 1 billion, about 1.5 billion or about 2 billion viable CD11b⁺ HLA-DR⁺ monocyte/macrophage.

In a preferred embodiment, the monocyte/macrophage composition and/or preparation is frozen or cryopreserved. In another embodiment, the monocyte/macrophage composition and/or preparation is fresh, i.e., the cells have not been previously frozen prior to expansion or cryopreservation. The terms “frozen/freezing” and “cryopreserved/cryopreserving/cryopreservation” are used interchangeably in the present application. Cryopreservation can be by any method known in the art that preserves cells in viable form. The freezing of cells is ordinarily destructive because on cooling, water within the cell freezes, leading to injury caused by osmotic effects on the cell membrane, cell dehydration, solute concentration, and ice crystal formation. As ice forms outside the cell, available water is removed from solution and withdrawn from the cell, causing osmotic dehydration and raised solute concentration which eventually destroys the cell. For a discussion, see Mazur, P., Cryobiology 14:251-272, 1977.

These injurious effects can be circumvented by (a) use of a cryoprotective agent, (b) control of the freezing rate, and (c) storage at a temperature sufficiently low to minimize degradative reactions.

Cryoprotective agents which can be used include, but are not limited to, dimethyl sulfoxide (DMSO) (Lovelock and Bishop, Nature 183:1394-1395, 1959; Ashwood-Smith, Nature 190:1204-1205, 1961); glycerol, polyvinylpyrrolidine (Rinfret, Ann. N.Y. Acad. Sci. 85:576, 1960); polyethylene glycol (Sloviter and Ravdin, Nature 196:548, 1962); albumin, dextran, sucrose, ethylene glycol, i-erythritol, D-ribitol, D-mannitol (Rowe et al., Fed. Proc. 21:157, 1962); D-sorbitol, i-inositol, D-lactose, choline chloride (Bender et al., J. Appl. Physiol. 15:520, 1960); amino acids (Phan The Tran and Bender, 1960, Exp. Cell Res. 20:651, 1960); methanol, acetamide, glycerol monoacetate (Lovelock, Biochem. J. 56:265, 1954); inorganic salts (Phan The Tran and Bender, Proc. Soc. Exp. Biol. Med. 104:388, 1960; Phan The Tran and Bender, in Radiobiology, Proceedings of the Third Australian Conference on Radiobiology, Ilbery ed., Butterworth, London, p. 59, 1961), and CryoStor® CS5 or CS10 (BioLife Solutions Inc., Bothell, WA). In a preferred embodiment, DMSO is used. For example, DMSO is used at a concentration which is nontoxic to cells. Additionally, DMSO comprises up to about 20% of the composition, up to about 15% of the composition, up to about 10% of the composition, up to about 5% of the composition, up to about 2% of the composition, up to about 1% of the composition, or up to about 0.5% of the composition. In some embodiments, addition of plasma (e.g., up to a concentration of about 20 to 25%) can augment the protective effect of DMSO. In some embodiments, addition of a human protein, such as for example, human serum albumin (e.g., up to a concentration of about 2 to 10%) can augment the protective effect of DMSO. After addition of DMSO, cells should be kept at 0 C until freezing, since DMSO concentrations of about 1% can be toxic at temperatures above 4 °C.

In another embodiment, PBS containing 20% DMSO and 8% human serum albumin (HSA), or other suitable cell freezing media is used. This mixture is then diluted 1:1 with media so that the final concentration of DMSO and HSA are 10% and 4%, respectively. The cells in this mixture are then frozen to −80 °C at a rate of 1 °C per minute and stored in the vapor phase of a liquid nitrogen storage tank.

A controlled slow cooling rate can be important. Different cryoprotective agents (Rapatz et al., Cryobiology 5 (1): 18-25, 1968) and different cell types have different optimal cooling rates (see e.g., Rowe and Rinfret, Blood 20:636, 1962; Rowe, Cryobiology 3 (1): 12-18, 1966; Lewis, et al., Transfusion 7 (1): 17-32, 1967; and Mazur, Science 168:939-949, 1970 for effects of cooling velocity on survival of marrow-stem cells and on their transplantation potential). The heat of fusion phase where water turns to ice should be kept to a minimum. The cooling procedure can be carried out by use of, e.g., a programmable freezing device or a methanol bath procedure known in the art.

A programmable freezing apparatus allows the determination of optimal cooling rates and facilitates standard reproducible cooling. A programmable controlled-rate freezer such as Cryomed or Planar permit tuning of the freezing regimen to the desired cooling rate curve. For example, for marrow cells in 10% DMSO and 20% plasma, the optimal rate is 1 °C to 3 °C per minute from 0 °C to 80 °C. In a preferred embodiment, this cooling rate of 1 °C to 3 °C per minute from 0 °C to 80 °C can be used. The container holding the cells must be stable at cryogenic temperatures and allow for rapid heat transfer for effective control of both freezing and thawing. Sealed plastic vials (e.g., Nunc, the Wheaton Cryule®) or glass ampules can be used for multiple small amounts (1 to 2 mL) or larger amounts (e.g., 5 to 30 mL), while larger volumes (20 to 200 mL) can be frozen in polyolefin bags (e.g., Del-Med) or ethylene vinyl acetate freezer bags (e.g., OriGen) held between metal plates for better heat transfer during cooling. By way of example, bags of bone marrow cells have been successfully frozen by placing them in −80 °C freezers which gives a cooling rate of approximately 3 °C/minute.

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred embodiment, DMSO-treated cells are pre-cooled on ice and transferred to a tray containing chilled methanol, which is placed in a mechanical refrigerator (e.g., Harris or Revco) at −80 °C. Thermocouple measurements of the methanol bath and the samples indicate the desired cooling rate of 1 °C to 3 °C per minute. After at least two hours, the specimens have reached a temperature of −80 °C and can be placed directly into liquid nitrogen (−196 °C) for permanent storage.

After thorough freezing, the monocyte/macrophage composition and/or preparation can be rapidly transferred to a long-term cryogenic storage vessel. In a preferred embodiment, samples are cryogenically stored in liquid nitrogen (−196 °C) or its vapor (between about −140 °C and −180 °C). In another preferred embodiment, samples are cryogenically stored in liquid nitrogen vapor phase (e.g., at about −140 °C to −180 °C). Such storage is greatly facilitated by the availability of highly efficient liquid nitrogen refrigerators, which resemble large Thermos® containers with an extremely low vacuum and internal super insulation, such that heat leakage and nitrogen losses are minimized.

Suitable racking systems are commercially available and can be used for cataloguing, storage, and retrieval of individual specimens.

Other methods of cryopreservation of viable cells, or modifications thereof, are available and envisioned for use (e.g., cold metal-mirror techniques; Livesey and Linner, Nature 327:255, 1987; Linner et al., J. Histochem. Cytochem. 34 (9): 1123-1135, 1986; see also U.S. Pat. No. 4,199,022 by Senkan et al., U.S. Pat. No. 3,753,357 by Schwartz, U.S. Pat. No. 4,559,298 by Fahy).

Cryopreserved or frozen cells are preferably thawed quickly (e.g., in a water bath maintained at 37 °C to 41 °C), and chilled immediately upon thawing. In a specific embodiment, the vial containing the frozen cells can be immersed up to its neck in a warm water bath with gentle rotation to ensure mixing of the cell suspension as it thaws and to increase heat transfer from the warm water to the internal ice mass. As soon as the ice has completely melted, the vial is immediately placed in ice.

In an embodiment, a cryopreserved monocyte/macrophage composition and/or preparation is thawed, and the full preparation, or a portion thereof, is infused into a human or animal patient in need thereof (e.g., having an infection, for example, a viral infection (e.g., HIV, HSV1 or 1, Hepatitis A, B, or C, Zika, SARS-CoV or SARS-CoV-2, and the like); or other infection as disclosed herein). Several procedures relating to processing of the thawed cells are available, known in the art, and can be employed if desirable.

It can be desirable to treat the cells to prevent cellular clumping upon thawing. To prevent clumping, various procedures are well known in the art and can be used in the disclosed methods, including, but not limited to, the addition of DNase before and/or after freezing (Spitzer et al., Cancer 45:3075-3085, 1980), low molecular weight dextran and citrate, and/or hydroxyethyl starch (Stiff et al., Cryobiology 20:17-24, 1983), and the like.

The cryoprotective agent, if toxic in humans, should be removed prior to therapeutic use of the thawed monocyte/macrophage composition and/or preparation. In an embodiment employing DMSO as the cryopreservative, it is preferable to omit this step to avoid cell loss. However, where removal of the cryoprotective agent is desired, the removal is preferably accomplished upon thawing.

For example, removal of the cryoprotective agent can be by dilution to achieve the cryoprotective agent at an insignificant concentration. This can be accomplished by an addition of medium, followed by, if necessary, one or more cycles of centrifugation to pellet cells, removal of the supernatant, and resuspension of the cells. For example, intracellular DMSO in the thawed cells can be reduced to a level (less than 1%) that will not adversely affect the recovered cells. This is preferably done slowly to minimize potentially damaging osmotic gradients that occur during DMSO removal.

After removal of the cryoprotective agent, cell count (e.g., by use of a hemocytometer) and viability testing (e.g., by trypan blue exclusion; Kuchler, Biochemical Methods in Cell Culture and Virology, Dowden, Hutchinson & Ross, Stroudsburg, Pa., pp. 18-19, 1977; Methods in Medical Research, Eisen et al., eds., Vol. 10, Year Book Medical Publishers, Inc., Chicago, pp. 39-47, 1964) can be performed to confirm cell survival. The percentage of viable antigen (e.g., CD11b⁺ HLA-DR⁺) positive cells can be determined by calculating the number of antigen positive cells that exclude 7-AAD (or other suitable dye excluded by viable cells) in an aliquot of the cells, divided by the total number of nucleated cells (TNC) (both viable and non-viable) in the aliquot of the cells. The number of viable antigen positive cells can then be determined by multiplying the percentage of viable antigen positive cells by the TNC.

Prior to cryopreservation and/or after thawing, the total number of nucleated cells, or in a specific embodiment, the total number of CD11b⁺ HLA-DR⁺ cells, can be determined. For example, total nucleated cell count can be performed by using a hemocytometer and exclusion of trypan blue dye. Specimens that are of high cellularity can be diluted to a concentration range appropriate for manual counting. Final cell counts for products are corrected for any dilution factors.

Total nucleated cell count equals viable nucleated cells per mL×volume of product in milliliters (mL). The number of CD11b⁺ HLA-DR⁺ positive cells in the sample can be determined, e.g., by use of flow cytometry using anti-CD11b and anti-HLA-DR monoclonal antibodies conjugated to at least one fluorochrome.

The monocyte/macrophage compositions and/or preparations can be used in immunotherapy for the treatment of viral disorders, bacterial infections, solid tumors, hematopoietic malignancies, autoimmune disorders, and the like. In some embodiments, the monocyte/macrophage compositions and/or preparations are administered to inhibit inflammation, viral/bacterial proliferation in a subject (also referred to as a patient) in need thereof. A method according to this aspect of the present invention is affected by administering a therapeutically effective amount of a monocyte/macrophage composition and/or preparation to the subject. As used herein, “treating” or “treatment” includes, but is not limited to, the administration of a monocyte/macrophage composition and/or preparation to reduce or delay the onset of the symptoms, complications, or biochemical indicia of a disease, alleviating the symptoms or arresting or inhibiting further development of the disease, condition, or disorder (e.g., cancer, metastatic cancer, metastatic solid tumors, viral or bacterial symptoms). Treatment can be prophylactic, i.e., as an adjuvant (to prevent relapse or delay the onset of the disease, or to prevent the manifestation of clinical or subclinical symptoms thereof) or therapeutic suppression or alleviation of symptoms after the manifestation of the disease.

In one embodiment, a monocyte/macrophage cell composition and/or preparation is administered in an amount effective to inhibit inflammation, inhibit viral and/or bacterial infection, or inhibit tumor proliferation.

While illustrative embodiments have been illustrated and described, it will be appreciated that various changes can be made therein without departing from the spirit and scope of the invention.

### EXAMPLES

### Example 1

The following example demonstrates the ex vivo expansion and differentiation of a monocyte and/or macrophage product from hematopoietic stem cells and/or hematopoietic progenitor cells (HSPCs). The HSPCs in this example are CD34⁺ cells isolated from human cord blood and/or human placental blood. The method comprises three distinct linear phases of ex vivo culture, where Phase 1 comprises Expansion, Phase 2 comprises Expansion and Monocyte Differentiation, and Phase 3 comprises Macrophage Differentiation.

### Phase 1—Expansion

CD34⁺ HSPCs, enriched from 2 to 20 pooled unmatched cord blood units, were seeded into tissue culture treated plastic culture vessels lacking any additional coating at a density of 9,000-10,000 cells/cm². The ex vivo culture medium used in the Phase 1 culturing step comprises X-VIVO™-10 medium (Lonza) supplemented with stem cell factor (SCF; 50 ng/mL), thrombopoietin (TPO; 50 ng/mL), FMS-like tyrosine kinase 3 ligand (Flt3-L; 50 ng/mL), and interleukin 6 (IL-6; 50 ng/ml), and interleukin 3 (IL-3; 10 ng/ml). Cells were cultured for 14 to 21 days, with either a feed with Phase 1 medium every 3 to 4 days or a harvest and replating of the cells in new vessels at a density of 20,000-30,000 cells/cm² on Day 7 and Day 14 (for the 21-day expansion) with additions of Phase 1 medium to accommodate increasing cell densities. Cell harvesting consisted of collection of non-adherent cells with the culture medium into a sterile conical tube. Adherent cells were incubated for 20 to 30 minutes with phosphate buffered saline (PBS), dislodged from the tissue culture plastic by pipetting, and combined with the non-adherent cells. All collected cells were then washed with PBS by centrifugation. Cells collected at the end of Phase 1 of culture could be either passaged directly into Phase 2 Expansion and Monocyte Differentiation or cryopreserved for later use using CryoStor® CS10 (Biolife Solutions) which comprises 10% DMSO as the cryopreservation medium.

### Phase 2—Expansion and Monocyte Differentiation

At the end of the Phase 1 HSPC Expansion, cells were harvested and washed with phosphate buffered saline (PBS) by centrifugation, and resuspended in Phase 2 culture medium, comprising of X-VIVO™-10 medium (Lonza) supplemented with monocyte colony stimulating factor (M-CSF; 50 ng/ml), Flt3-L (50 ng/ml), and IL-3 (10 ng/mL). Alternately, cells cryopreserved at the end of Phase 1 of culture were thawed and resuspended in Phase 2 culture medium.

Cells were seeded into tissue culture treated plastic culture vessels lacking any additional coating at a density of 20,000-30,000 cells/cm². Cells were cultured for 7 days with a feed with Phase 2 medium 3 to 4 days into the culture period. Cell harvesting at the end of Phase 2 of culture consisted of collection of non-adherent cells with the culture medium into a sterile conical tube. Adherent cells were incubated for 20-30 minutes with phosphate buffered saline (PBS), dislodged from the tissue culture plastic by pipetting, and combined with the non-adherent cells. All collected cells were then washed with PBS by centrifugation. Cells collected at the end of Phase 2 of culture could be either passaged directly into Phase 3 Macrophage Differentiation culture or cryopreserved for later use using CryoStor® CS10 (Biolife Solutions) as the cryopreservation medium.

### Phase 3—Macrophage Differentiation

At the end of the Phase 2 Expansion and Monocyte Differentiation culture, cells were harvested and washed with PBS by centrifugation, and resuspended in Phase 3 culture medium, comprising of RPMI 1640 (Gibco), supplemented with granulocyte-macrophage colony stimulating factor (GM-CSF; 10 ng/mL) and either 2.5% human platelet lysate (HPL) or 10% fetal bovine serum (FBS). Alternately, cells cryopreserved at the end of Phase 2 culture were thawed and resuspended in Phase 3 culture medium. Cells were seeded into tissue culture treated plastic culture vessels lacking any additional coating at a density of 130,000-230,000 cells/cm². Cells were cultured for 7 days with a feed with Phase 3 medium 3 to 4 days into the culture period. At the end of Phase 3 culture, non-adherent cells were collected with the culture medium into a sterile conical tube. Adherent cells were incubated with TrypLE™ Express Enzyme (Gibco) according to product instructions, followed by scraping of the tissue culture plastic surface, and the released adherent cells were combined with the collected non-adherent cells and washed with RPMI 1640 by centrifugation. Washed cells could either be used for in vitro or in vivo analyses or cryopreserved for later use using CryoStor® CS10 (Biolife Solutions) as the cryopreservation medium.

### Results

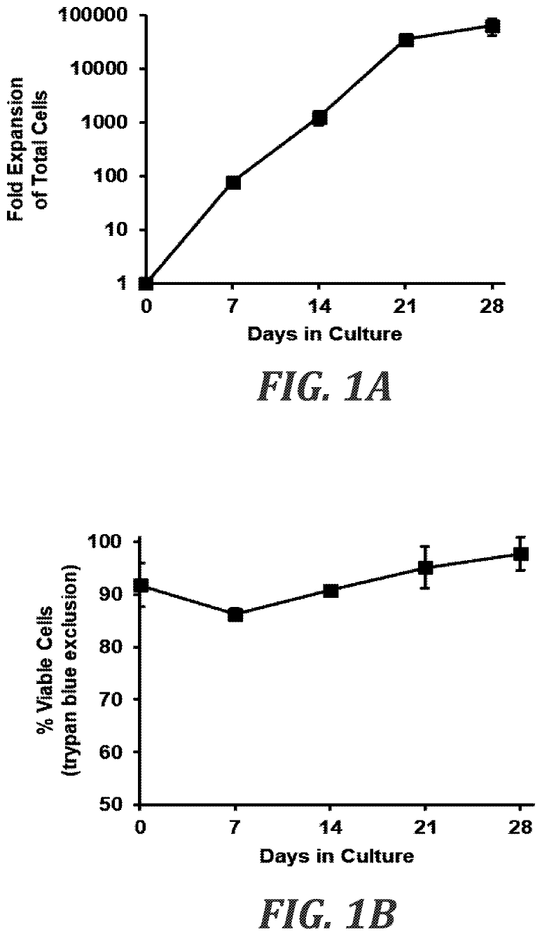

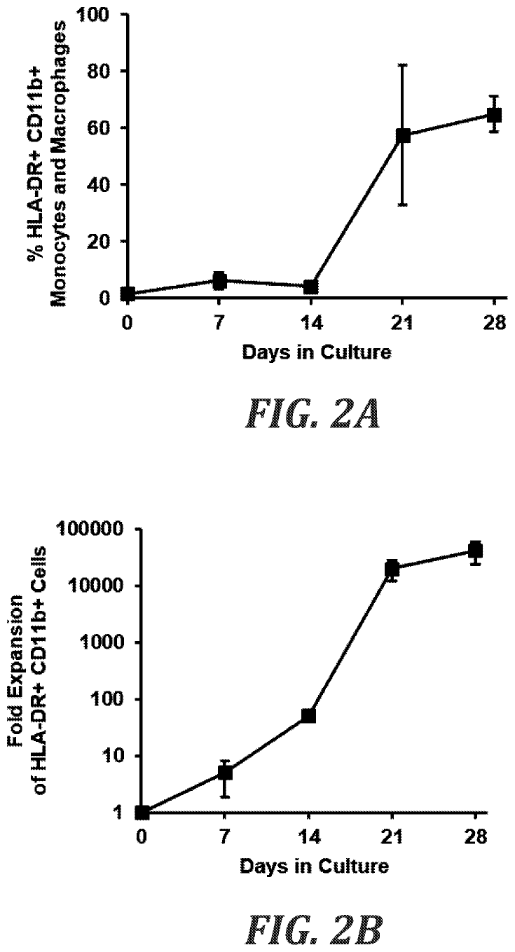

Ex vivo expansion of cells during Phase 1, Phase 2, and Phase 3 of culture generates approximately 78,000 cells from each starting CD34⁺ cell seeded into culture, with greater than 80% viability maintained throughout the culture process (see FIGS. 1A and 1B). Directed differentiation of the expanded cell population that occurs during Phase 2 and Phase 3 of culture results in 60 to 90% of the cells being identified as monocytes or macrophages, generating approximately 54,000 monocytes and macrophages from each starting CD34⁺ cell seeded into culture (see FIGS. 2A and 2B).

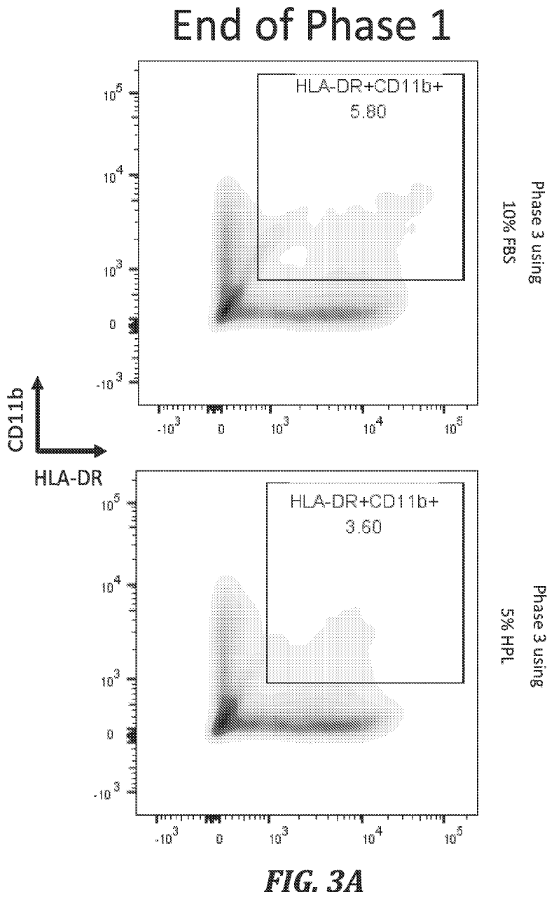

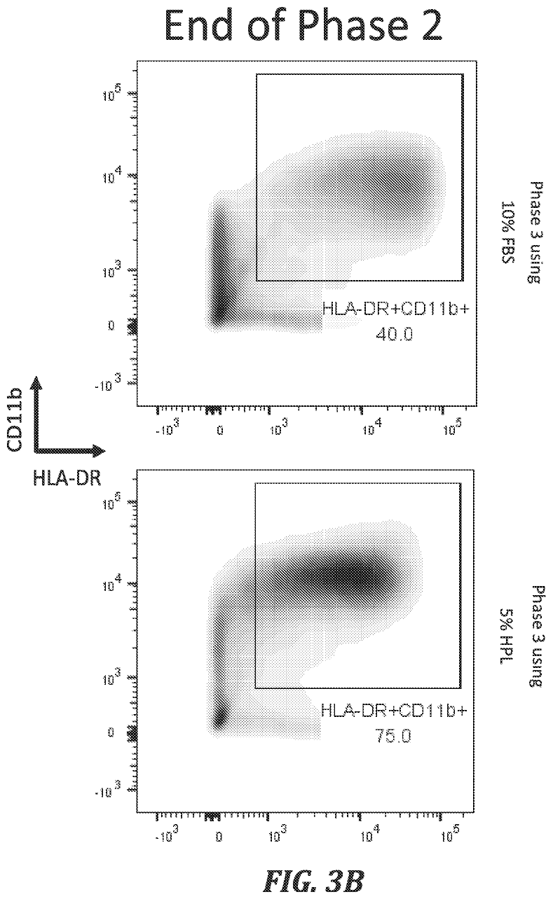

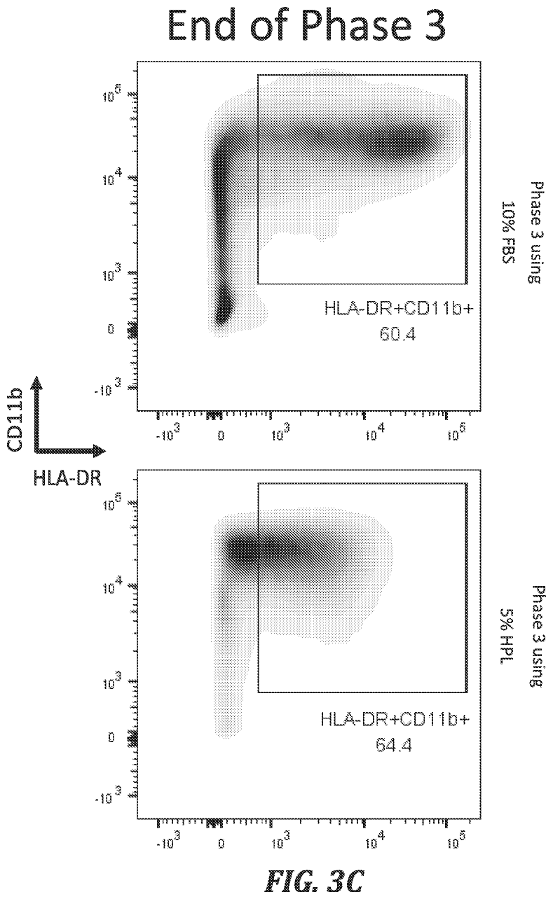

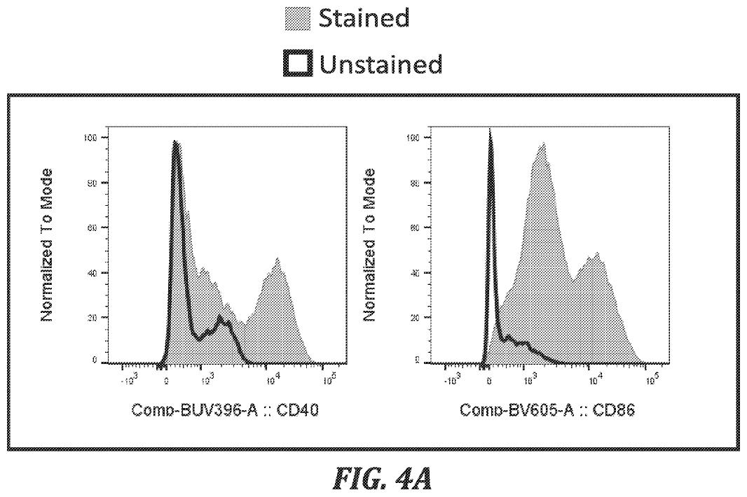

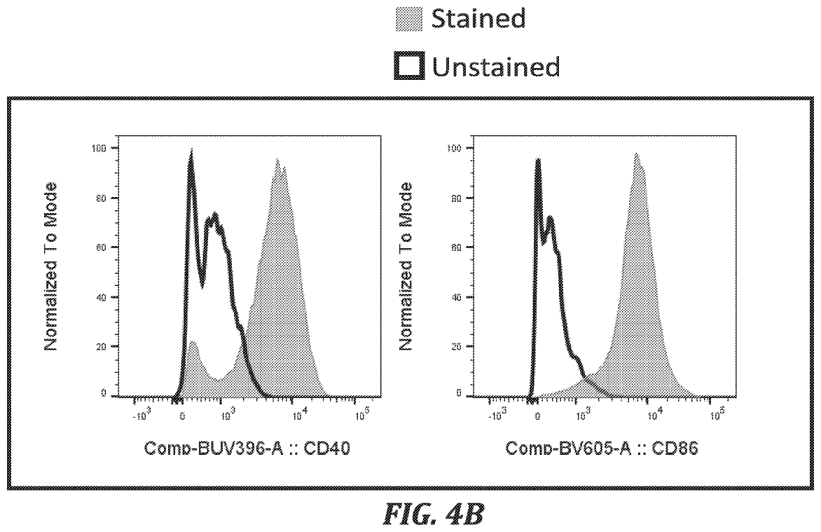

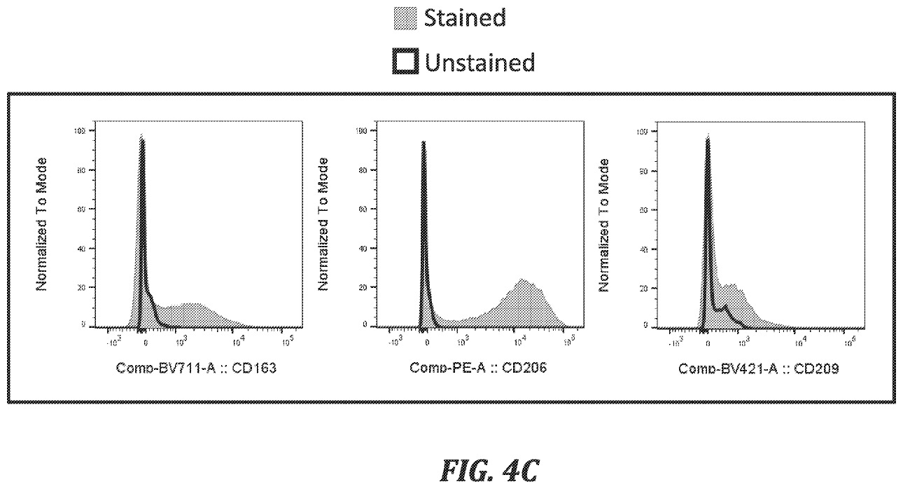

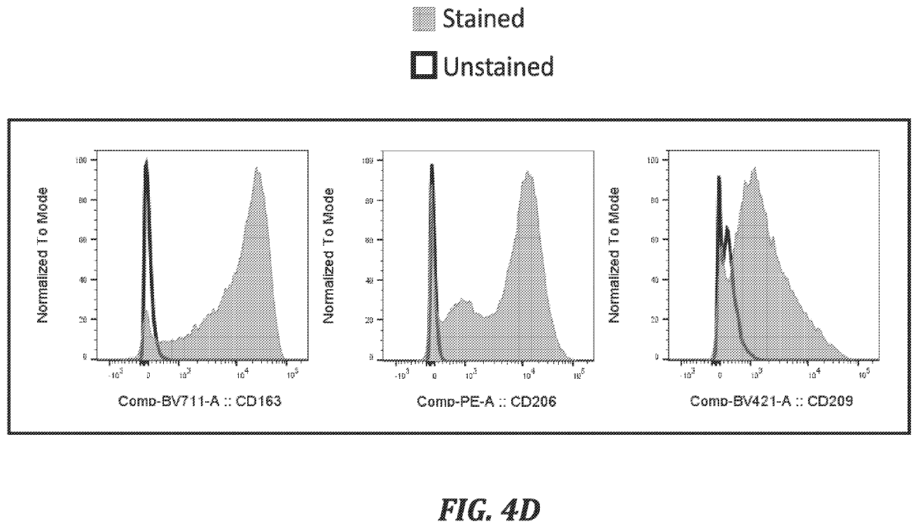

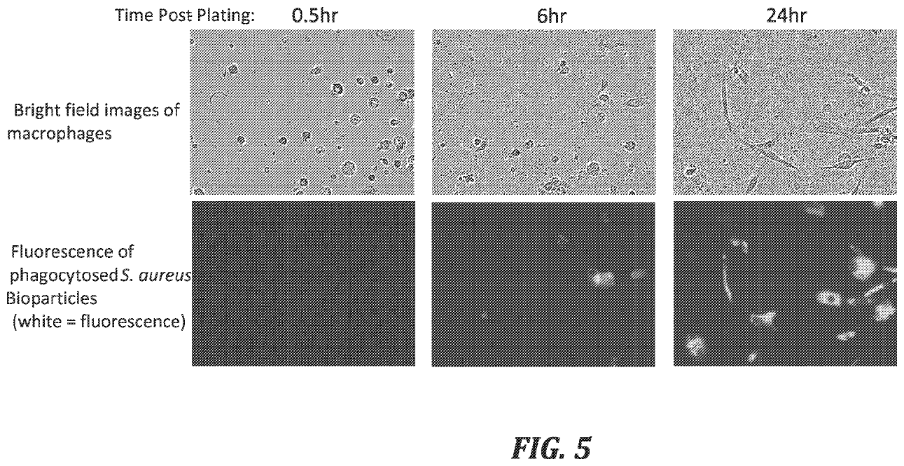

Monocytes generated above express the cell surface proteins HLA-DR and CD11b as well as the cell surface receptors CD14 and/or CD16. At the end of the Expansion and Monocyte Differentiation phase of culture, the HLA-DR⁺ CD11b⁺ monocyte population makes up 40 to 80% of the total cell population (see FIGS. 2A and 2B and FIGS. 3A and 3B). At the end of the Expansion and Monocyte Differentiation (Phase 2 of culture), an average of 4.1%+/−4.1% of the total cells are CD14⁺ CD16⁻, classified as Classical monocytes, an average of 66.7%+/−10.8% of the total cells are CD14⁺ CD16⁺, classified as Intermediate monocytes, and an average of 8.4%+/−6.9% of the total cells are CD14^{low} CD16⁺, classified as Non-Classical monocytes. Macrophages differentiated from the monocytes generated through the current invention also express the cell surface proteins HLA-DR and CD11b as well as the cell surface receptors CD14 and/or CD16. At the end of the Macrophage Differentiation (Phase 3 of culture), the HLA-DR⁺ CD11b⁺ macrophage population makes up 60-90% of the total cell population (see FIGS. 2A and 2B and FIGS. 3A-3C). The total cell population at the end of the Macrophage Differentiation (Phase 3 of culture) expresses CD14 and CD16 comparable to that observed at the end of the Expansion and Monocyte Differentiation (Phase 2 of culture) (7.2%+/−7.2% CD14⁺ CD16−; 66.9%+/−11.5% CD14⁺ CD16⁺; 5.1%+/−3.1% CD14⁻ CD16⁺). The remaining cell population that is not HLA-DR⁺ CD11b⁺ is comprised of myeloid-derived cells, including dendritic cells. Depending upon whether granulocyte-macrophage colony stimulating factor (GM-CSF) or macrophage colony stimulating factor (M-CSF) is used to differentiate the monocytes to macrophages, additional markers are expressed on these macrophages that are associated with either an M1 polarization (e.g., CD40, CD86) or an M2 polarization (e.g., CD163, CD206, CD209), respectively (see FIGS. 4A-4D).

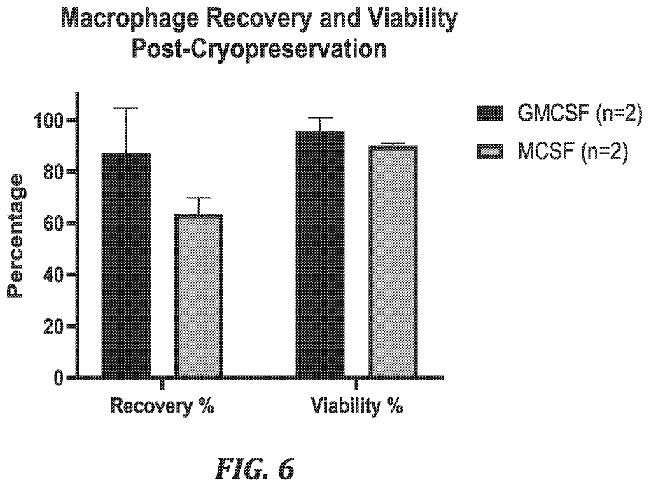

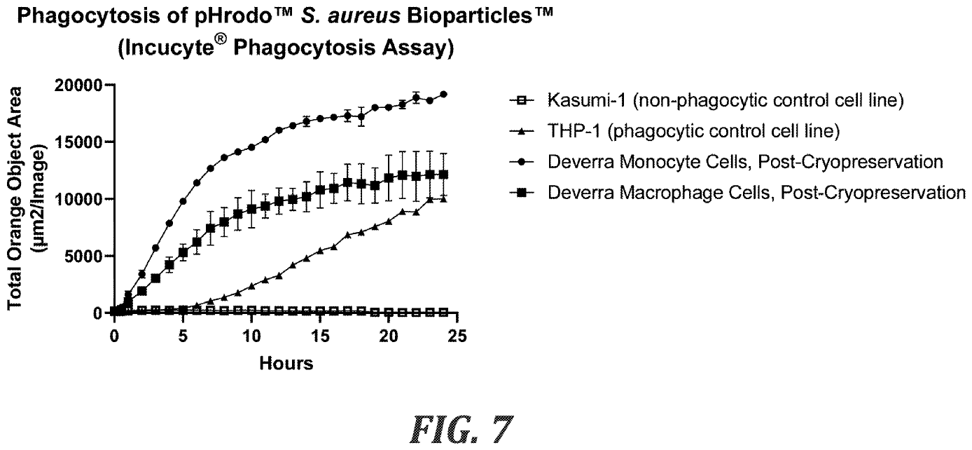

Both monocytes and macrophages generated through the current methods are capable of phagocytosis, as demonstrated by in vitro phagocytosis assays using the pH—sensitive pHrodo™ S. aureus Bioparticles™ that become fluorescent within the acidic environment of the cytoplasmic phagosome (see FIG. 5). Both monocytes and macrophages can be cryopreserved, with high levels of cell recovery and viability post-thaw (see FIG. 6). Cryopreserved monocytes and macrophages also retain their capacity for phagocytosis following thaw (see FIG. 7). This demonstrated phagocytosis can include traditional macrophage targets like bacteria and other pathogens, dead cells and debris, and tumor cells. When these macrophages are activated by pathogens or tumor cells, or encounter dead cells and debris during wound healing, they can release cytokines associated with either M1 or M2 polarization, such as Tumor Necrosis Factor-alpha (TNFα) and IL-12 by activated M1 macrophages or IL-10 by immunomodulatory M2 macrophages, for example.

### Example 2

The following example demonstrates that a CD19 CAR-expressing monocytes and/or macrophages can be generated from hematopoietic stem cells and/or hematopoietic progenitor cells (HSPCs) that were engineered to express a CD19 CAR construct using viral transduction. The HSPCs in this example are CD34⁺ cells isolated from human cord blood and/or human placental blood. The method comprises the three distinct linear phases of ex vivo culture as described in Example 1, modified to include viral transduction of HSPCs during the Phase 1 Expansion culture as described below.

CD34⁺ HSPCs were initiated into culture as described in Example 1. On day 2 of culture, the Phase 1 media overlying the cells was replaced with CD19 CAR viral vector-conditioned media in a volume sufficient to completely submerge the cells. The tissue culture vessel containing the cells and vector-conditioned media was then centrifuged at 1400×g for 1 hour at room temperature (~25 °C). Immediately following centrifugation, the tissue culture vessel was incubated for 2 hours in a humidified incubator set to 37 °C and 5% CO₂. The transduction process was then repeated by replacing the spent vector-conditioned media with fresh vector-conditioned media as described above, and the tissue culture vessel containing the cells and fresh vector-conditioned media was centrifuged at 1400×g for 1 hour at room temperature (~25 °C). Immediately following the second centrifugation, the tissue culture vessel was incubated for 1 hour in a humidified incubator set to 37 °C and 5% CO₂. Following this incubation, the spent vector-conditioned media was replaced with fresh Phase 1 media, and the tissue culture vessel was returned to a humidified incubator set to 37 °C and 5% CO₂. The remainder of the Phase 1 Expansion, Phase 2 Expansion and Monocyte Differentiation, and Phase 3 Macrophage Differentiation cultures proceeded as described in Example 1. Cells were collected and cryopreserved at the end of Phase 2 and the end of Phase 3 of culture for subsequent in vitro analyses.

### Results

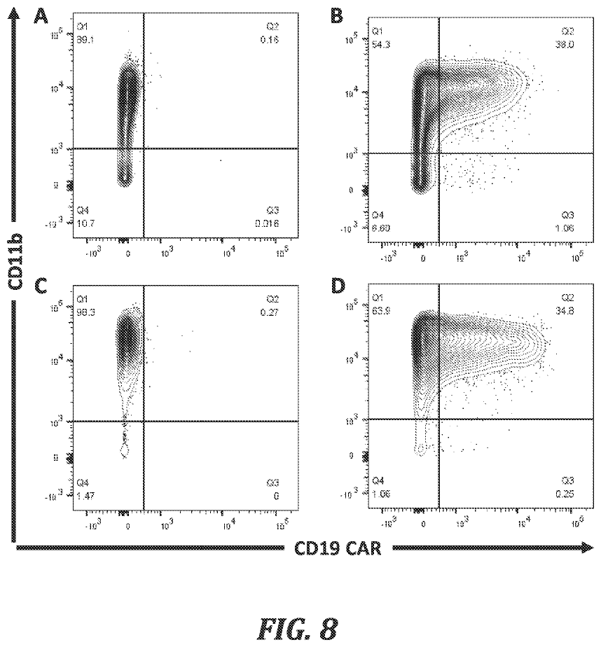

Monocytes and macrophages generated by ex vivo expansion and differentiation of CD19 CAR-engineered HSPCs express cell surface CD19 CAR following cryopreservation and thaw of the cells (see FIGS. 8A-8D). Within the cells collected and cryopreserved at the end of the Phase 2 Expansion and Monocyte Differentiation culture, the CD19 CAR⁺ CD11b⁺ monocyte population makes up 38% of the total cell population (see FIGS. 8A and 8B). Within the cells collected and cryopreserved at the end of the Phase 3 Macrophage Differentiation culture, the CD19 CAR⁺ CD11b⁺ macrophage population makes up 34.8% of the total cell population (see FIGS. 8C and 8D).

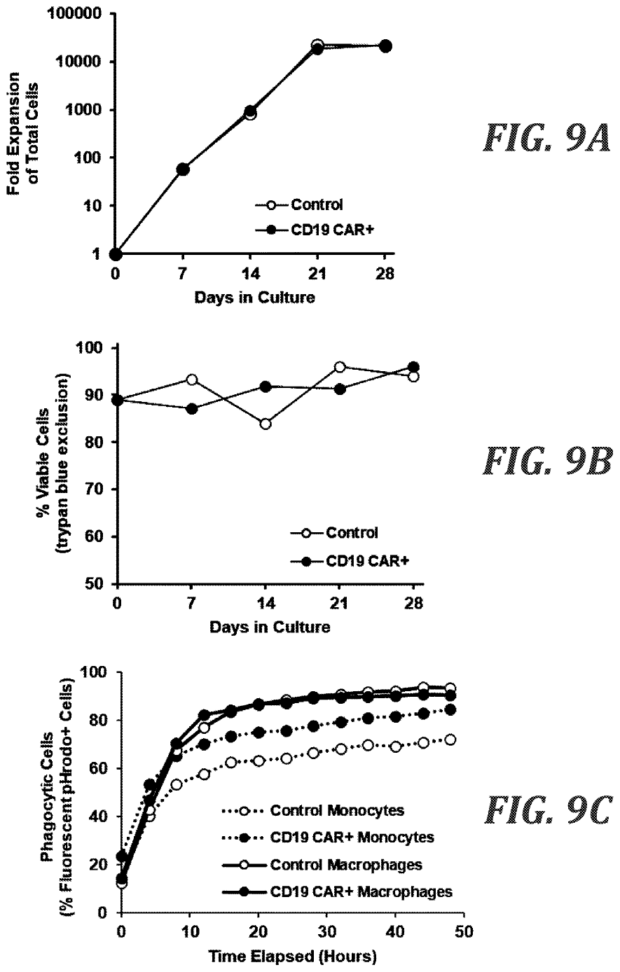

The cell engineering process and expression of the CD19 CAR by the cells do not reduce the total fold expansion (see FIG. 9A) or viability (see FIG. 9B) of the cells compared with control untransduced cells. Cryopreserved and thawed monocytes and macrophages expressing a cell surface CD19 CAR retain their capacity for phagocytosis at similar or improved levels compared with control untransduced monocytes and macrophages (see FIG. 9C).

The Range of Culture Conditions Tested that Generate Monocytes and Macrophages from Cord Blood CD34⁺ Cells

### CD34⁺ Cell Source.

The CD34⁺ cell source can comprise single cord blood units from non-HLA matched (unmatched), matched, or partially mismatched donors.
Alternately, the CD34⁺ cell source can comprise pooled cord blood units from unmatched, matched, or partially mismatched donors.
The number of pooled donor cord blood units can comprise between 2 and 20, or more.
Enriched CD34⁺ cells can be used fresh, immediately following enrichment, or can comprise cryopreserved and stored in liquid nitrogen vapor phase for use at a later date.

### Duration of the Culture Phases

The duration of the Phase 1 HSPC Expansion culture can comprise between 3 and 21 days, such as for example, 3 days, 7 days, 10 days, 14 days, or 21 days.
The duration of the Phase 2 Expansion and Monocyte Differentiation culture can comprise between 7 and 14 days, such as, for example, 7 days, 10 days, or 14 days.
The duration of the Phase 3 Macrophage Differentiation culture can comprise 6 to 8 days.
The preferred culture condition comprises a 14-to-21-day Phase 1 culture duration, a 7-day Phase 2 culture duration, and a 7-day Phase 3 culture duration.

### Culture Media Base Formulations

Base medium used during Phase 1 HSPC Expansion culture can comprise StemSpan™ SFEM II, StemPro™-34 SFM, X-VIVO™-10, X-VIVO™-15, PRIME-XV Expansion XSFM, CellGenix® SCGM, StemLine® or StemLine® II Hematopoietic Stem Cell Expansion Medium, or StemMACS HSC Expansion Media.
Base medium used during Phase 2 Expansion and Monocyte Differentiation culture can comprise StemSpan™ SFEM II, StemPro™-34 SFM, X-VIVO™-10, X-VIVO™-15, or IMDM.
Base medium used during Phase 3 Macrophage Differentiation culture can comprise X-VIVO™ 10, X-VIVO™ 15, RPMI 1640, IMDM, or ImmunoCult™-SF Macrophage Medium.
The preferred culture condition comprises use of X-VIVO™-10 during Phase 1 of culture, X-VIVO™ 10 during Phase 2 of culture, and RPMI 1640 during Phase 3 of culture.

### Culture Media Supplements

Culture media supplements can comprise serum or a serum replacement and/or one or more cytokines.
Cytokine supplements used during Phase 1 HSPC Expansion culture comprise 50 ng/ml stem cell factor (SCF), 50 ng/mL thrombopoietin (TPO), 50 ng/mL FMS-like tyrosine kinase 3 ligand (Flt3-L), 50 ng/mL interleukin 6 (IL-6), and 10 ng/mL interleukin 3 (IL-3).
Cytokine supplements used during Phase 2 Expansion and Monocyte Differentiation culture can comprise a combination of 50 ng/mL M-CSF, 50 ng/mL Flt3-L, and 10 ng/ml interleukin 3 (IL-3).
Cytokine supplements used during Phase 2 Expansion and Monocyte Differentiation may alternately comprise a combination of 50 ng/ml M-CSF, 50 ng/mL SCF, 50 ng/mL TPO, 50 ng/mL Flt3-L, 50 ng/mL IL-6, and 10 ng/ml IL-3.
Cytokine supplements used during Phase 2 Expansion and Monocyte Differentiation can alternately comprise a combination of 50 ng/ml M-CSF, 50 ng/ml Flt3-L, 10 ng/mL IL-3, and 50 ng/mL IL-6; it can alternately comprise a combination of 50 ng/mL GM-CSF, 50 ng/ml MCSF, 50 ng/mL Flt3-L, and 10 ng/ml IL-3.
Serum or serum replacements used during Phase 3 Macrophage Differentiation culture can comprise, for example, 10% fetal bovine serum (FBS); 10% human AB serum; 2.5% human platelet lysate (HPL), 5% HPL, or 10% HPL; or 0.25% human serum albumin (HSA), 0.5% HSA, 1% HSA, or 2% HSA.
Cytokine supplements used during Phase 3 Macrophage Differentiation culture can also comprise, for example, 10 ng/mL GM-CSF, 25 ng/mL GM-CSF, or 50 ng/ml GM-CSF; or 25 ng/mL M-CSF, 50 ng/mL M-CSF, or 100 ng/ml M-CSF.
The preferred culture conditions comprise X-VIVO™-10 medium supplemented with 50 ng/mL stem cell factor (SCF), 50 ng/mL thrombopoietin (TPO), 50 ng/ml FMS-like tyrosine kinase 3 ligand (Flt3-L), 50 ng/mL interleukin 6 (IL-6), and 10 ng/ml interleukin 3 (IL-3) for Phase 1 HSPC Expansion; X-VIVO™-10 medium supplemented with 50 ng/ml MCSF, 50 ng/mL Flt3-L, and 10 ng/ml IL-3 for Phase 2 Expansion and Monocyte Differentiation; and RPMI 1640 medium supplemented with 10 ng/mL GM-CSF and either 2.5% HPL or 10% FBS for Phase 3 Macrophage Differentiation.

### Tissue Culture Substrates or Coatings

In vitro culture of differentiating cells with feeder cell layers or with a tissue culture vessel substrate or coating may be done to activate cell signaling pathways that influence cell differentiation toward a specific desired lineage or activation status.
During the Phase 1 HSPC Expansion culture, cells have been cultured in tissue culture treated plastic culture vessels:
   without additional coating or pre-treatment;
   pre-coated with 0.1 μg/cm² Delta1^{extIgG} (DXI) and 0.8 μg/cm² recombinant human fibronectin fragment (RetroNectin®);
   pre-coated with 0.16 μg/cm² DXI and 0.8 μg/cm² RetroNectin®;
   pre-coated with 0.2 μg/cm² DXI and 0.8 μg/cm² RetroNectin®; and
   pre-coated with 0.4 μg/cm² DXI and 0.8 μg/cm² RetroNectin®
During the Phase 1 HSPC Expansion culture, cells have been cultured in the presence of no coating or DXI and RetroNectin coating:
   for all 7 days of a 7-day Phase 1 culture period;
   for the first 7 days of a 14- or 21-day Phase 1 culture period; and
   for the first 14 days of a 21-day Phase 1 culture period.
During the Phase 2 Expansion and Monocyte Differentiation culture, cells have been cultured in tissue culture treated plastic culture vessels:
   without additional coating or pre-treatment;
   pre-coated with 0.1 μg/cm² DXI and 0.8 μg/cm² RetroNectin®;
   pre-coated with 0.2 μg/cm² DXI and 0.8 μg/cm² RetroNectin®; and
   pre-coated with 0.4 μg/cm² DXI and 0.8 μg/cm² RetroNectin®
During the Phase 3 Macrophage Differentiation, cells have been cultured in tissue culture treated plastic culture vessels without additional coating or pre-treatment.
The preferred culture condition comprises tissue culture treated plastic culture vessels with DXI coating for 7 days, 10 days, or 14 days, followed by 7 days without DXI coating for Phase 1 of culture, and using tissue culture treated plastic culture vessels without coating or pre-treatment for Phase 2 and Phase 3 of culture; an alternate culture condition comprises tissue culture treated plastic culture vessels without additional coating or pre-treatment during Phase 1, Phase 2, and Phase 3 of culture.

### Cryopreservation of Monocytes and Macrophages

The cells generated through the above are intended for use either as a fresh cell product that has not been cryopreserved or as a cryopreserved cell product. Cells can be cryopreserved following the completion of the first phase of culture, at which point expanded stem and progenitor cells have been generated. A cryopreserved expanded cell product can differentiate to monocytes and macrophages upon thaw and culture under the differentiation culture conditions described herein. Alternately, the cell product can be used as a fresh monocyte cell product or as a cryopreserved monocyte cell product following the completion of the second phase of culture, at which point differentiated monocytes have been generated. A cryopreserved monocyte cell product can differentiate to functional macrophages and dendritic cells upon thaw. Alternately, the cell product can be used as a fresh macrophage cell product or as a cryopreserved macrophage cell product following the completion of the third phase of culture, at which point differentiated macrophages have been generated.

Both monocytes and macrophages generated through the above can be cryopreserved and thawed with recovery of about 46 to about 85% of the monocytes with about 66 to about 90% viability and recovery of about 53 to about 99% of the macrophages with about 83 to about 99% viability. Monocytes and macrophages can be cryopreserved using CryoStor® CS10, and in the alternative the cells can also be effectively cryopreserved using similar cryopreservation media including, but not limited to, CryoStor® CS5, FBS+5% DMSO, FBS+10% DMSO, human AB serum+5% DMSO, human AB serum+10% DMSO, HPL+5% DMSO, HPL+10% DMSO.

1. A method of preparing a monocyte and/or macrophage preparation for use in immunotherapy, comprising:
selecting a plurality of umbilical cord blood or placental blood cells;
preparing enriched CD34⁺ hematopoietic stem and progenitor cells (HSPCs) that are depleted of red blood cells and T cells;
culturing the CD34⁺ enriched HSPCs in an expansion culture medium comprising interleukin-3 (IL-3), interleukin-6 (IL-6), thrombopoietin (TPO), Flt-3 ligand (Flt-3L), and stem cell factor (SCF) on a solid phase for a sufficient time to produce expanded HSPCs, wherein the expanded HSPCs do not substantially differentiate into CD11b⁺ HLA-DR⁺ cells during the expansion; and
culturing the expanded HSPCs in an expansion and monocyte differentiation culture medium comprising effective amounts of Flt-3 ligand (Flt-3L), interleukin 3 (IL-3), and monocyte colony stimulating factor (M-CSF) and/or granulocyte-macrophage colony stimulating factor (GM-CSF) on a solid phase for a sufficient time to produce expanded HSPCs and monocytes, the cell composition comprising an average of about 40 to about 80% HLA-DR⁺ CD11b⁺ cells and an average of about 20 to about 60% other myeloid cells, the cell population also comprising about 70% CD14⁺ CD16⁺ cells;
culturing the expanded HSPCs and monocytes in a macrophage differentiation culture medium comprising effective amounts of granulocyte-macrophage colony stimulating factor or M-CSF and serum or a non-animal sourced serum replacement for a sufficient time to produce a macrophage cell composition and/or preparation comprising about 60 to about 90% HLA-DR⁺ CD11b⁺ cells and an average of about 10 to about 40% other myeloid cells, the cell population also comprising about 7% CD14⁺ CD16⁻ cells, about 66% CD14⁺ CD16⁺ cells, and about 5% CD14⁻ CD16⁺ cells; and
wherein the macrophages can be activated by pathogens or tumor cells.

2. The method of claim 1, wherein the umbilical cord blood or placental blood cells are from a non-HLA matched, matched, or partially mismatched donor.

3. The method of claim 1, wherein the umbilical cord blood or placental blood cells are pooled from non-HLA matched, matched, or partially mismatched donors.

4. The method of any of the preceding claims, wherein the expansion culture further comprises a Notch ligand and fibronectin.

5. The method of any of the preceding claims, wherein the expansion culture is carried out for about 3 to about 21 days.

6. The method of any of the preceding claims, wherein the expansion and monocyte differentiation culture is carried out for about 7 to about 14 days.

7. The method of any of the preceding claims, wherein the macrophage differentiation phase is carried out for about 6 to 8 days.

8. The method of any of the preceding claims, wherein the non-animal serum replacement is human AB serum, human serum albumin, or human platelet lysate.

9. The method of any of the preceding claims, wherein the HSPCs are not derived from somatic cells, embryonic stem cells, peripheral blood mononuclear cells or induced pluripotent stem cells.

10. The method of any of the preceding claims, wherein the Notch ligand is DXI or an antibody specific for Notch.

11. The method of any of the preceding claims, wherein cells of the monocyte and/or macrophage composition and/or preparation are genetically modified.

12. The method of claim 11, wherein the genetic modification is during the expansion phase or subsequent to differentiation of the HSPCs, monocytes and/or macrophage.

13. The method of any one of claim 11 or 12, wherein the cells of the monocyte and/or macrophage composition and/or preparation are genetically modified to affect a gene knockdown, knockout, or knock-in.

14. The method of claim 13, wherein the gene targeted for knockdown or knockout is B2M, CIITA, a cytokine, a cytokine receptor, a chemokine, a chemokine receptor, a transcription factor, a growth factor receptor, an immunomodulatory molecule, an immune stimulatory molecule, an immune costimulatory molecule, and/or an immune costimulatory ligand.

15. The method of claim 13, wherein the gene targeted for knock-in is HLA-E, HLA-E and B2M, a cytokine, a cytokine receptor, a chemokine, a chemokine receptor, a transcription factor, a growth factor receptor, an immunoregulatory molecule, an immune stimulatory molecule, an immune costimulatory molecule, and/or an immune costimulatory ligand.

16. The method of any one of claims 11 to 15, wherein the cells of the monocyte and/or macrophage composition and/or preparation are genetically modified to express an RNA, an enzyme, a receptor, a chimeric receptor, an immune cell engager, an antibody, a nanobody, a transcription factor, a cytokine and/or a chemokine.

17. The method of claim 16, wherein the RNA is a shRNA, a siRNA or a gRNA.

18. The method of claim 16, wherein the receptor is a CAR or a TCR.

19. The method of any one of claims 16 to 18, wherein the receptor, chimeric receptor, immune cell engager, antibody, or nanobody specifically bind to a viral antigen, a bacterial antigen, a tumor-specific, a tumor-associated, or stroma antigen.

20. The method of claim 19, wherein the viral antigen is present in a Cytomegalovirus (CMV), an Epstein Barr Virus (EBV), a Human Immunodeficiency Virus (HIV), a Herpes simplex virus (HSV), a Hepatitis virus, a Zika virus, an influenza virus, or a coronavirus.

21. The method of claim 20, wherein the Herpes virus is HSV 1 or HSV 2, the Hepatitis virus is Hepatitis A, B, or C, and the coronavirus is SARS-CoV or SARS-CoV-2.

22. The method of claim 19, wherein the tumor-specific antigen, tumor-associated antigen, or stroma antigen is AFP, ALPP, ALPP2, ANTXR1, alpha-V beta-3 integrin, alpha-V beta-6 integrin, AXL, BCMA, B7-H3 (CD276), B7-H4 (VTCN1), carbonic anhydrase IX (CA1X), carcinoembryonic antigen (CEA), CD5, CD8, CD7, CD10, CD19, CD20, CD22, CD30, CD33, CD34, CD38, CD41, CD44, CD44V6, CD47, CD49c, CD49e, CD49f, CD56, CD61, CD66c, CD70, CD72, CD73, CD74, CD80, CD86, CD104,CD123, CD126, CD133, CD138, CD142, CD147, CD318, an antigen of a cytomegalovirus (CMV) infected cell (e.g., a cell surface antigen), cutaneous lymphocyte-associated antigen (CLA; a specialized glycoform of P-selectin glycoprotein ligand-1 (PSGL-1)), a chlorotoxin ligand, claudin 6 (CLDN6) claudin 18.2 (CLDN18.2), CLL1 CRLF2, DLL-3, DR4, DR5, EGF1R, epidermal growth factor receptor (EGFR), EGFR806, EGFRvIII, epithelial glycoprotein-2 (EGP-2), epithelial glycoprotein-40 (EGP-40), epithelial cell adhesion molecule (EpCAM), EpHA2, receptor tyrosine-protein kinases erb-B2,3,4 (erb-B2,3,4), ERBB, ERBB2, folate-binding protein (FBP), fetal acetylcholine receptor (AChR), FAP, folate receptor-alpha (FOLR1), FZD1, FZD2, FZD3, FZD4, FZD5, FZD6, FZD7, FZD8, FZD9, FZD10, Ganglioside G2 (GD2), Ganglioside G3 (GD3), GFRA4, GP100, GPC2, GPC3, GSPG4, GUCY2C, human Epidermal Growth Factor Receptor 2 (HER2), human telomerase reverse transcriptase (hTERT), ICAM1 (CD54), Interleukin-13 receptor subunit alpha-2 (IL-13Ralpha2), kappa-light chain, kinase insert domain receptor (KDR), KLK2, Lewis Y (LeY), L1 cell adhesion molecule (L1CAM; CD171), LMP1, LRRC15, melanoma antigen family A, 1 (MAGE-A1), MAGEA3, MAGEA4, MART1, mesothelin (MSLN), MET (c-Met; HGFR), MG7, mucin 1 (MUC1), TnMUC1, MUC3A, mucin 16 (MUC16), NECTIN4, NKG2D, an NKG2D ligand (for example, MIC-A, MIC-B, ULBP1, ULBP2, ULBP3, ULBP4, ULBP5, ULBP6), cancer-testis antigen NY-ESO-1, oncofetal antigen (h5T4), p53, PD-1, PD-L1, PD-L2, Proteinase3 (PR1), PRAME, prostate stem cell antigen (PSCA), prostate-specific membrane antigen (PSMA), REG3A (PAP), ROR1, ROR2, Survivin, Tyrosinase, tumor-associated glycoprotein 72 (TAG-72), TROP2, tetraspanin 8 (TSPAN8), vascular endothelial growth factor R2 (VEGF-R2), or Wilms tumor protein (WT-1).

23. The method of claim 22, wherein the tumor-specific or tumor-associated antigen is EGFR or any variant thereof, a NKG2D ligand, HER2, B7-H3, PSMA, PSCA, MUC1 or a variant thereof, mesothelin, or CEA.

24. The method of any of claims 18 to 23, wherein the CAR comprises an extracellular antigen binding domain, a hinge region, a transmembrane domain, and one or more intracellular signaling domains.

25. The method of claim 24, wherein the intracellular signaling domain comprises one or more cytoplasmic or intracellular signaling domains of CD3zeta (CD3ζ), CD3delta, CD3epsilon, CD3gamma, CD4, CD8A, CD8B, CD2, CD7, LIGHT, CD27, CD28, 4-1BB (CD137), CD226 (DNAM1), B24 (CD244), ICOS (CD278), CTLA-4, GITR, OX40 (CD134), LAT, PD-1, TIM3, TIGIT, PD-L1, PD-L2, OX40L, 4-1BBL, ICOSLG, CD30L, CD30, CD36, CD68, CD40, CD70, CD80, CD83, CD86, CD163, CD204 (MSR1), CD206 (MRC1), CD209 (DC-SIGN), AGER (RAGE), CD276 (B7-H3), CD147, LILRB1, LILRB2, LILRB3, LILRB4, LILRB5, LILRA1, LILRA2, LILRA3, LILRA4, LILRA5, LILRA6, CLEC1A (CLEC1), CLEC1B (CLEC2), CLEC2A, CLEC2B, CLEC4D (DECTIN-3), CLEC4E (MINCLE), CLEC5A, CLEC6A (DECTIN-2), CLEC7A (DECTIN-1), CLEC8A (LOX-1)., CLEC9A (DNGR-1), CLEC10A (MGL), CLEC12A (MICL), SIGLEC1-11, SIGLEC14-16, AXL, MERTK, TYRO3, TREM2, CD11A (LFA-1; ITGAL), CD11B (ITGAM), CD11C (ITGAX), CSF1R (M-CSFR; CD115), GM-CSFR (CD116), CD14, CD16A (FcγRIIIa), CD32A (FcγRIIa), CD32B (FcγRIIb), CD32C (FcγRIIc), CD64 (FcγRI), FcRγ (FcεRIγ), MEGF10, CD79A, CD79B, CD19, TLR-1, TLR-2, TLR-3, TLR-4, TLR-5, TLR-6, TLR-7, TLR-8, TLR-9, MYD88, MAL, TRAM (TICAM2), TRIF (TICAM1), DAP-10, DAP-12, MAVS, STING, RIG-I, AIM2, NOD1-5, NLRP1-14, RIPK2, CASP1-10, CASP12-L, CASP12-S, CASP-14, a cytokine receptor, IFNGR, IFNGR1, IFNGR2, IFNAR, IFNAR1, IFNAR2, IFNLR1, CD116 (GM-CSFR), CSF2RA, CSF2RB, CSF1R (CD115; M-CSFR), IL10R, IL10RA, IL10RB, TGFBR, TGFBR1, TGFBR2. TNFRSF1A, TNFRSF1B), a chemokine receptor comprising CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, XCR1, CX3CR1, or any combination thereof; a transmembrane domain comprising a transmembrane domain of an intracellular signaling domain, and optionally comprises CD8, CD28, CD3zeta, CD4, 4-1BB, OX40, ICOS, or NKG2D; and a spacer region comprising a hinge region of IgG1, the CH₂CH₃ region of an immunoglobulin, a portion of CD3, a portion of CD28, or a portion of CD8.

26. The method of claim 19, wherein the immune cell engager specifically binds to one or more T cell surface proteins, one or more NK cell surface proteins, or one or more monocyte and/or macrophage surface protein.

27. The method of claim 26, wherein the T cell surface protein is CD3, CD28, and/or 4-1BB.

28. The method of claim 26, wherein the NK cell surface protein is CD56, CD16, and/or NKG2D.

29. The method of claim 26, wherein the monocyte and/or macrophage surface protein is CD64, CD40, CD80, and/or CD86.

30. The method of claim 16, wherein the transcription factor is a C/EBP transcription factor, a NF-κB transcription factor, a STAT transcription factor, a KLF transcription factor, a PPAR transcription factor, an AP-1 transcription factor, a NFAT transcription factor, a GATA transcription factor, a CREB transcription factor, or an IRF transcription factor.

31. The method of claim 16, wherein the cytokine or chemokine is an Interleukin, Interferon α, Interferon β, Interferon γ, Interferon λ, TGF β, TNF α, TNF β, GM-CSF, M-CSF, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, XCL1, XCL2, CX3CL1, CXCL1, CXCL2, CXLC3, CXCL4, CXCL5, CXCL6, CXCL7, CXCL8, CXCL9, CXCL10, CXCL11, CXCL12, CXCL13, CXCL14, CXCL16, or CXCL17.

32. The method of claim 31, wherein the interleukin is IL-1 α, IL-1β, IL-2, IL-4, IL-5, IL-6, IL-7, IL-8, IL-9, IL-10, IL-11, IL-12, IL-13, IL-15, IL-17, IL-18, IL-21, or IL-22.

33. The method of any of the preceding claims, wherein the monocyte and/or macrophage composition and/or preparation further comprises a cryoprotective agent.

34. The method of any of the preceding claims, further comprising formulating the monocyte and/or macrophage composition and/or preparation to form a monocyte and/or macrophage formulation for infusion into a subject.

35. A composition comprising the monocyte and/or macrophage composition and/or preparation produced by any of the methods of the preceding claims for use in immunotherapy.

36. The composition of claim 35, wherein immunotherapy comprises use as a therapeutic agent against tumor cells, use as an antimicrobial agent, use for autoimmune indications, or use as a therapeutic in repair of injured tissue.

37. A composition comprising the monocyte and/or macrophage composition and/or preparation of any of the methods of the preceding claims for use in delivery of small molecules, plasmid DNA, oncolytic virus, or other therapeutics.

38. A composition comprising the monocyte and/or macrophage composition and/or preparation of any of the methods of the preceding claims for use in combination with other therapeutic compositions comprising unmodified or genetically modified T cells or NK cell therapies, antibody or nanobody therapeutics, immune cell engagers, cytokines, chemokines, and/or oncolytic virus.


SEQUENCE LISTING (75 sequences)

SEQ ID NO: 1 - protein, 11 aa, synthetic construct
RASQDISKYLN

SEQ ID NO: 2 - protein, 7 aa, synthetic construct
SRLHSGV

SEQ ID NO: 3 - protein, 9 aa, synthetic construct
GNTLPYTFG

SEQ ID NO: 4 - protein, 5 aa, synthetic construct
DYGVS

SEQ ID NO: 5 - protein, 16 aa, synthetic construct
VTWGSETTYYNSALKS

SEQ ID NO: 6 - protein, 7 aa, synthetic construct
YAMDYWG

SEQ ID NO: 7 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 8 - protein, 247 aa, Homo sapiens
MASPLTRELSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 9 - protein, 69 aa, synthetic construct
STTTKPVLRTPSPVHPTGTSQPQRPEDCRPRGSVKGTGLDFACDIYIWAPLAGICVALLLSLIITLICY

SEQ ID NO: 10 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 11 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - protein, 188 aa, Homo sapiens
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 13 - protein, 113 aa, Homo sapiens
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 14 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 15 - DNA, 767 bp, Homo sapiens
  source 1..767: mol_type = other DNA
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 16 - DNA, 601 bp, Homo sapiens
  source 1..601: mol_type = other DNA
atgaagtcaggcctctggtatttctttctcttctgcttgcgcattaaagttttaacaggagaaatcaatggttctgccaattatgagatgtttatatttcacaacggaggtgtacaaattttatgcaaatatcctgacattgtccagcaatttaaaatgcagttgctgaaaaggggggcaaatactctgcgatctcactaagacaaaaggaagtggaaacacagtgtccattaagagtctgaaattctgccattctcagttatccaacaacagtgtctctttttttctacaaccttggaccattctcatgccaactattacttctgcaacctatcaatttttgatcctcctccttttaaagtaactcttacaggaggatatttgcatatttatgaaatcacaactttgttgccagctgaagttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctataa

SEQ ID NO: 17 - DNA, 282 bp, Homo sapiens
  source 1..282: mol_type = other DNA
atgatccatctgggtcacatcctcttcctgcttttgctcccagtggctgcagctcagacgaccccaggagagagatcatcactccctgccttttaccctggcacttcaggctcctgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggctga

SEQ ID NO: 18 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 19 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 20 - protein, 199 aa, Homo sapiens
MKSGLWYFFLFCLRIKVLTGEINGSANYEMFIFHNGGVQILCKYPDIVQQFKMQLLKGGQILCDLIKTKGSGNTVSIKSLKFCHSQLSNNSVSFFLYNLDHSHANYYFCNLSIFDPPPFKVTLIGGYLHIYESQLCCQLKFWLPIGCAAFVVVCILGCILICWLTKKKYSSSVHDPNGEYMFMRAVNTAKKSRLTDVTL

SEQ ID NO: 21 - protein, 485 aa, synthetic construct
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 462 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRL

SEQ ID NO: 23 - protein, 491 aa, synthetic construct
MALPVTALLLPLALLLHAEVKLQESGGGFVKPGGSLKVSCAASGFTFSSYAMSWVRLSPEMRLEWVATISSAGGYIFYSDSVQGRFTISRDNAKNTLHLQMGSLRSGDTAMYYCARQGFGNYGDYYAMDYWGQGTTVTVSSGGGGSGGGGSGGGGSDIELTQSPSSLAVSAGEKVTMSCKSSQSLLNSRTRKNQLAWYQQKPGQSPELLIYWASTRQSGVPDRFTGSGSGTDFTLTISSVQAEDLAVYYCQQSYNLLTFGPGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 491 aa, synthetic construct
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 121 aa, synthetic construct
EVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSS

SEQ ID NO: 26 - protein, 115 aa, synthetic construct
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRAD

SEQ ID NO: 27 - protein, 116 aa, synthetic construct
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRSDP

SEQ ID NO: 28 - protein, 15 aa, synthetic construct
GGGGSGGGGSGGGGS

SEQ ID NO: 29 - protein, 117 aa, synthetic construct
EVQLVESGGGLVQPGNSLKLSCSASGFTFSSYGMHWIRQAPGEGLDWVAYISSSSGTVYADAVKARFTISRDNAKNTLYLQLNSLKSEDTAIYYCARAQNWDLFDYWGQGVMVTVSS

SEQ ID NO: 30 - protein, 112 aa, synthetic construct
QIMLTQQAESLWISPGERVSITCRASQSLLYTDGKHYLSWYQQKPGQTTKALIYHASVRTDGVPTRFIGSGSGTEFTLSIEHVQPEDFAIYYCLQTLKSPFTFGSGTKLEIK

SEQ ID NO: 31 - protein, 119 aa, synthetic construct
QVQLKQSGPGLVQPSQSLSITCTVSGFSLSNYGVHWVRQSPGKGLEWLGVIWSGGSTDYNVAFISRLIITKDNSKSQVFLKMNSLQADDTAIYFCARTTGSVFNAMDHWGQGTSVTVSS

SEQ ID NO: 32 - protein, 106 aa, synthetic construct
QIVLTQSPALMSASPGEKVTMTCSASSSVTYMYWYQQKPRSSPKPWIYLTSNLASGVPARFSGSGSGTSYSLTISSVEAEDAATYYCQQWSSNPLTFGAGTKLELK

SEQ ID NO: 33 - protein, 122 aa, synthetic construct
EVQLQQSGAELVKPGASVKLSCTASGFNIKDTYIHWVKQRPEQGLEWIGRIDPATGNTEYDPKFQGKATITADTSSNTAYLHLSSLTSEDTAVYYCARGYYGSSYPPWFAYWGQGTLVTVSA

SEQ ID NO: 34 - protein, 107 aa, synthetic construct
DIVMTQSHKFMSTSVGDRVSITCKASQDVGSAVAWYQQKPGQSPKLLIYWASTRHTGVPDRFTGSGSGTDFTLTISNVQSEDLADYFCQQYSSYPLTFGAGTKLELK

SEQ ID NO: 35 - protein, 117 aa, synthetic construct
EVQLQQSGAELVKPGASVKLSCTASGFNIKDTYMHWVKQRPEQGLEWIGRIDPANGHTKYDPKFQGKATITADTSSNAAYLQLNSLTSEDTAVYYCARRVAYAMDYWGQGTSVTVSS

SEQ ID NO: 36 - protein, 106 aa, synthetic construct
ENVLTQSPAIMSASPGEKVTMTCSASSSVTYMHWYQQKSSTSPKLWIYDTSKLASGVPGRFSGSGSGNSYSLTISSMEAEDVATYCCFQGSGYPLTFGGGTKLEIK

SEQ ID NO: 37 - protein, 118 aa, synthetic construct
QVTLKESGPGILKPSQTLSLTCSFSGFSLSTSGMGVGWIRQPSGKSLEWLAHIWWNDERYYNPSLKNQLTISKDTSRNQVFLKITSVDTADTATYYCARSPRGYFDYWGHGTTLTVSS

SEQ ID NO: 38 - protein, 107 aa, synthetic construct
DIVMTQSQKFMSTSVGDRVSVTCKASQNVVTNVAWYQQTPGQSPKALIYSASYRYSGVPDRFSGSGSGTDFTLTISNVQSGDLAEYFCQQYNSYPLTFGAGTKLELK

SEQ ID NO: 39 - protein, 120 aa, synthetic construct
QVNLLQSGAALVKPGASVKLSCKASGYTFTDYYIFWVKQSHGKSLEWIGYINPNSGSTNYNEKFKRKATLSVDKSTNTAYMELSRLTSEDSATYYCTRRAYYGYNPFDYWGQGVMVTVSS

SEQ ID NO: 40 - protein, 107 aa, synthetic construct
DIQMTQTPSSMPASLGERVTISCRASRGINNYLSWYQQNLDGTIKPLIYYTSNLQSGVPSRFSGSGSGTDYSLTISSLEPEDFAMYYCQQYDSSPWTFGGGTKLELK

SEQ ID NO: 41 - protein, 119 aa, synthetic construct
EVQLQQSGAELVRPGALVKLSCKASGFNIKDYYIHWVKQRPEQGLEWIGWIDPENGHTIYDPKFQGKASITADTSSNTAYLQLSSLTSEDTAVYYCARLTGTTYAMDYWGQGTSVTVSS

SEQ ID NO: 42 - protein, 107 aa, synthetic construct
DIVMTQSHKFMSTSVGDRVSITCKASQDVSTAVAWYQQKSGQSPKLLIYWASTRHTGVPDRFTGSGSGTDYTLTISSVQAEDLALYYCQQHYSTPYTFGGGTKLEIK

SEQ ID NO: 43 - protein, 123 aa, synthetic construct
EVKLLESGGGLVQPGGSMRLSCAASGFTFTDFYMNWIRQPAGKAPEWLGFIRNKASGYTTEYNPSVKGRFTISRDNTQNMLYLQMNTLRAEDTATYYCARAHSYYGYDYFDYWGQGVMVTVSS

SEQ ID NO: 44 - protein, 107 aa, synthetic construct
DIQMTQSPASLSASLEEIVTITCQASQDIGNWLSWYQQKPGKSPQLLIYGATSLADGVPSRFSGSRSGTQYSLKISRLQVEDIRIYYCLQAYSAPWTFGGGTKLELK

SEQ ID NO: 45 - protein, 244 aa, synthetic construct
EVQLVESGGGLVQPGNSLKLSCSASGFTFSSYGMHWIRQAPGEGLDWVAYISSSSGTVYADAVKARFTISRDNAKNTLYLQLNSLKSEDTAIYYCARAQNWDLFDYWGQGVMVTVSSGGGGSGGGGSGGGGSQIMLTQQAESLWISPGERVSITCRASQSLLYTDGKHYLSWYQQKPGQTTKALIYHASVRTDGVPTRFIGSGSGTEFTLSIEHVQPEDFAIYYCLQTLKSPFTFGSGTKLEIK

SEQ ID NO: 46 - protein, 244 aa, synthetic construct
QIMLTQQAESLWISPGERVSITCRASQSLLYTDGKHYLSWYQQKPGQTTKALIYHASVRTDGVPTRFIGSGSGTEFTLSIEHVQPEDFAIYYCLQTLKSPFTFGSGTKLEIKGGGGSGGGGSGGGGSEVQLVESGGGLVQPGNSLKLSCSASGFTFSSYGMHWIRQAPGEGLDWVAYISSSSGTVYADAVKARFTISRDNAKNTLYLQLNSLKSEDTAIYYCARAQNWDLFDYWGQGVMVTVSS

SEQ ID NO: 47 - protein, 240 aa, synthetic construct
QVQLKQSGPGLVQPSQSLSITCTVSGFSLSNYGVHWVRQSPGKGLEWLGVIWSGGSTDYNVAFISRLIITKDNSKSQVFLKMNSLQADDTAIYFCARTTGSVFNAMDHWGQGTSVTVSSGGGGSGGGGSGGGGSQIVLTQSPALMSASPGEKVTMTCSASSSVTYMYWYQQKPRSSPKPWIYLTSNLASGVPARFSGSGSGTSYSLTISSVEAEDAATYYCQQWSSNPLTFGAGTKLELK

SEQ ID NO: 48 - protein, 240 aa, synthetic construct
QIVLTQSPALMSASPGEKVTMTCSASSSVTYMYWYQQKPRSSPKPWIYLTSNLASGVPARFSGSGSGTSYSLTISSVEAEDAATYYCQQWSSNPLTFGAGTKLELKGGGGSGGGGSGGGGSQVQLKQSGPGLVQPSQSLSITCTVSGFSLSNYGVHWVRQSPGKGLEWLGVIWSGGSTDYNVAFISRLIITKDNSKSQVFLKMNSLQADDTAIYFCARTTGSVFNAMDHWGQGTSVTVSS

SEQ ID NO: 49 - protein, 244 aa, synthetic construct
EVQLQQSGAELVKPGASVKLSCTASGFNIKDTYIHWVKQRPEQGLEWIGRIDPATGNTEYDPKFQGKATITADTSSNTAYLHLSSLTSEDTAVYYCARGYYGSSYPPWFAYWGQGTLVTVSAGGGGSGGGGSGGGGSDIVMTQSHKFMSTSVGDRVSITCKASQDVGSAVAWYQQKPGQSPKLLIYWASTRHTGVPDRFTGSGSGTDFTLTISNVQSEDLADYFCQQYSSYPLTFGAGTKLELK

SEQ ID NO: 50 - protein, 244 aa, synthetic construct
DIVMTQSHKFMSTSVGDRVSITCKASQDVGSAVAWYQQKPGQSPKLLIYWASTRHTGVPDRFTGSGSGTDFTLTISNVQSEDLADYFCQQYSSYPLTFGAGTKLELKGGGGSGGGGSGGGGSEVQLQQSGAELVKPGASVKLSCTASGFNIKDTYIHWVKQRPEQGLEWIGRIDPATGNTEYDPKFQGKATITADTSSNTAYLHLSSLTSEDTAVYYCARGYYGSSYPPWFAYWGQGTLVTVSA

SEQ ID NO: 51 - protein, 238 aa, synthetic construct
EVQLQQSGAELVKPGASVKLSCTASGFNIKDTYMHWVKQRPEQGLEWIGRIDPANGHTKYDPKFQGKATITADTSSNAAYLQLNSLTSEDTAVYYCARRVAYAMDYWGQGTSVTVSSGGGGSGGGGSGGGGSENVLTQSPAIMSASPGEKVTMTCSASSSVTYMHWYQQKSSTSPKLWIYDTSKLASGVPGRFSGSGSGNSYSLTISSMEAEDVATYCCFQGSGYPLTFGGGTKLEIK

SEQ ID NO: 52 - protein, 238 aa, synthetic construct
ENVLTQSPAIMSASPGEKVTMTCSASSSVTYMHWYQQKSSTSPKLWIYDTSKLASGVPGRFSGSGSGNSYSLTISSMEAEDVATYCCFQGSGYPLTFGGGTKLEIKGGGGSGGGGSGGGGSEVQLQQSGAELVKPGASVKLSCTASGFNIKDTYMHWVKQRPEQGLEWIGRIDPANGHTKYDPKFQGKATITADTSSNAAYLQLNSLTSEDTAVYYCARRVAYAMDYWGQGTSVTVSS

SEQ ID NO: 53 - protein, 240 aa, synthetic construct
QVTLKESGPGILKPSQTLSLTCSFSGFSLSTSGMGVGWIRQPSGKSLEWLAHIWWNDERYYNPSLKNQLTISKDTSRNQVFLKITSVDTADTATYYCARSPRGYFDYWGHGTTLTVSSGGGGSGGGGSGGGGSDIVMTQSQKFMSTSVGDRVSVTCKASQNVVTNVAWYQQTPGQSPKALIYSASYRYSGVPDRFSGSGSGTDFTLTISNVQSGDLAEYFCQQYNSYPLTFGAGTKLELK

SEQ ID NO: 54 - protein, 240 aa, synthetic construct
DIVMTQSQKFMSTSVGDRVSVTCKASQNVVTNVAWYQQTPGQSPKALIYSASYRYSGVPDRFSGSGSGTDFTLTISNVQSGDLAEYFCQQYNSYPLTFGAGTKLELKGGGGSGGGGSGGGGSQVTLKESGPGILKPSQTLSLTCSFSGFSLSTSGMGVGWIRQPSGKSLEWLAHIWWNDERYYNPSLKNQLTISKDTSRNQVFLKITSVDTADTATYYCARSPRGYFDYWGHGTTLTVSS

SEQ ID NO: 55 - protein, 242 aa, synthetic construct
QVNLLQSGAALVKPGASVKLSCKASGYTFTDYYIFWVKQSHGKSLEWIGYINPNSGSTNYNEKFKRKATLSVDKSTNTAYMELSRLTSEDSATYYCTRRAYYGYNPFDYWGQGVMVTVSSGGGGSGGGGSGGGGSDIQMTQTPSSMPASLGERVTISCRASRGINNYLSWYQQNLDGTIKPLIYYTSNLQSGVPSRFSGSGSGTDYSLTISSLEPEDFAMYYCQQYDSSPWTFGGGTKLELK

SEQ ID NO: 56 - protein, 242 aa, synthetic construct
DIQMTQTPSSMPASLGERVTISCRASRGINNYLSWYQQNLDGTIKPLIYYTSNLQSGVPSRFSGSGSGTDYSLTISSLEPEDFAMYYCQQYDSSPWTFGGGTKLELKGGGGSGGGGSGGGGSQVNLLQSGAALVKPGASVKLSCKASGYTFTDYYIFWVKQSHGKSLEWIGYINPNSGSTNYNEKFKRKATLSVDKSTNTAYMELSRLTSEDSATYYCTRRAYYGYNPFDYWGQGVMVTVSS

SEQ ID NO: 57 - protein, 241 aa, synthetic construct
EVQLQQSGAELVRPGALVKLSCKASGFNIKDYYIHWVKQRPEQGLEWIGWIDPENGHTIYDPKFQGKASITADTSSNTAYLQLSSLTSEDTAVYYCARLTGTTYAMDYWGQGTSVTVSSGGGGSGGGGSGGGGSDIVMTQSHKFMSTSVGDRVSITCKASQDVSTAVAWYQQKSGQSPKLLIYWASTRHTGVPDRFTGSGSGTDYTLTISSVQAEDLALYYCQQHYSTPYTFGGGTKLEIK

SEQ ID NO: 58 - protein, 241 aa, synthetic construct
DIVMTQSHKFMSTSVGDRVSITCKASQDVSTAVAWYQQKSGQSPKLLIYWASTRHTGVPDRFTGSGSGTDYTLTISSVQAEDLALYYCQQHYSTPYTFGGGTKLEIKGGGGSGGGGSGGGGSEVQLQQSGAELVRPGALVKLSCKASGFNIKDYYIHWVKQRPEQGLEWIGWIDPENGHTIYDPKFQGKASITADTSSNTAYLQLSSLTSEDTAVYYCARLTGTTYAMDYWGQGTSVTVSS

SEQ ID NO: 59 - protein, 245 aa, synthetic construct
EVKLLESGGGLVQPGGSMRLSCAASGFTFTDFYMNWIRQPAGKAPEWLGFIRNKASGYTTEYNPSVKGRFTISRDNTQNMLYLQMNTLRAEDTATYYCARAHSYYGYDYFDYWGQGVMVTVSSGGGGSGGGGSGGGGSDIQMTQSPASLSASLEEIVTITCQASQDIGNWLSWYQQKPGKSPQLLIYGATSLADGVPSRFSGSRSGTQYSLKISRLQVEDIRIYYCLQAYSAPWTFGGGTKLELK

SEQ ID NO: 60 - protein, 245 aa, synthetic construct
DIQMTQSPASLSASLEEIVTITCQASQDIGNWLSWYQQKPGKSPQLLIYGATSLADGVPSRFSGSRSGTQYSLKISRLQVEDIRIYYCLQAYSAPWTFGGGTKLELKGGGGSGGGGSGGGGSEVKLLESGGGLVQPGGSMRLSCAASGFTFTDFYMNWIRQPAGKAPEWLGFIRNKASGYTTEYNPSVKGRFTISRDNTQNMLYLQMNTLRAEDTATYYCARAHSYYGYDYFDYWGQGVMVTVSS

SEQ ID NO: 61 - protein, 842 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 62 - protein, 647 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 63 - protein, 6 aa, synthetic construct
DNSKSQ

SEQ ID NO: 64 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 65 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 66 - protein, 216 aa, Homo sapiens
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 67 - protein, 335 aa, synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 68 - protein, 161 aa, Homo sapiens
RISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 69 - DNA, 1943 bp, synthetic construct
  source 1..1943: mol_type = other DNA
atgctgctgctggtgaccagcctgctgctgtgcgagctgccccaccccgcctttcgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgagggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcggatttccaaacctcacctgcgctctatctctatccagtgctatctgtgcctgctgctgaactcacatttcctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatcagcctggagagcggcgacgcctctatccacgataccgtggagaacctgatcatcctggccaacaatagcctgagcagcaacggcaatgtgacagagtccggctgcaaggagtgtgaggagctggaggagaagaatatcaaagagttcctgcagtcattcgtccatatcgtccagatgtttatcaatacctcctaa

SEQ ID NO: 70 - protein, 725 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 71 - DNA, 2178 bp, synthetic construct
  source 1..2178: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgtggaggaggaagaggaaggagaagcagagcgagacaagccctaaggagtttctgacaatctatgaagacgtgaaggacctgaagacacggagaaaccacgagcaggagcagaccttccctggaggaggcagcacaatctactccatgatccagtctcagagcagcgcccccacctcccaggagcctgcctacacactgtatagcctgatccagccatcccggaagtctggcagcaggaagcgcaaccactccccctcttttaattctaccatctatgaagtgatcggcaagagccagcccaaggcacagaaccccgcacgactgagcaggaaggaactggagaactttgatgtctactctagggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcggatttccaaacctcacctgcgctctatctctatccagtgctatctgtgcctgctgctgaactcacatttcctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatcagcctggagagcggcgacgcctctatccacgataccgtggagaacctgatcatcctggccaacaatagcctgagcagcaacggcaatgtgacagagtccggctgcaaggagtgtgaggagctggaggagaagaatatcaaagagttcctgcagtcattcgtccatatcgtccagatgtttatcaatacctcctaa

SEQ ID NO: 72 - protein, 639 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPPFFFCCFIAVAMGIRFIIMVTKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPRLEGGGEGRGSLLTCGDVEENPGPRMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 73 - DNA, 1923 bp, synthetic construct
  source 1..1923: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctcccttctttttctgctgttttatcgccgtggctatgggcatccggttcatcatcatggtgaccaaacggggcagaaagaaactcctgtatatattcaaacaaccatttatgagaccagtacaaactactcaagaggaagatggctgtagctgccgatttccagaagaagaagaaggaggatgtgaactgagggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcggatttccaaacctcacctgcgctctatctctatccagtgctatctgtgcctgctgctgaactcacatttcctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatcagcctggagagcggcgacgcctctatccacgataccgtggagaacctgatcatcctggccaacaatagcctgagcagcaacggcaatgtgacagagtccggctgcaaggagtgtgaggagctggaggagaagaatatcaaagagttcctgcagtcattcgtccatatcgtccagatgtttatcaatacctcctaa

SEQ ID NO: 74 - protein, 718 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 75 - DNA, 2157 bp, synthetic construct
  source 1..2157: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctcccttctttttctgctgttttatcgccgtggctatgggcatccggttcatcatcatggtgacctggaggaggaagaggaaggagaagcagagcgagacaagccctaaggagtttctgacaatctatgaagacgtgaaggacctgaagacacggagaaaccacgagcaggagcagaccttccctggaggaggcagcacaatctactccatgatccagtctcagagcagcgcccccacctcccaggagcctgcctacacactgtatagcctgatccagccatcccggaagtctggcagcaggaagcgcaaccactccccctcttttaattctaccatctatgaagtgatcggcaagagccagcccaaggcacagaaccccgcacgactgagcaggaaggaactggagaactttgatgtctactctagggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcggatttccaaacctcacctgcgctctatctctatccagtgctatctgtgcctgctgctgaactcacatttcctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatcagcctggagagcggcgacgcctctatccacgataccgtggagaacctgatcatcctggccaacaatagcctgagcagcaacggcaatgtgacagagtccggctgcaaggagtgtgaggagctggaggagaagaatatcaaagagttcctgcagtcattcgtccatatcgtccagatgtttatcaatacctcctaa